(19) United States
(12) Patent Application Publication
(10) Pub. No.: US 2020/0239897 A1
(43) Pub. Date: Jul. 30, 2020

(54) PROMOTERS FROM CORYNEBACTERIUM GLUTAMICUM AND USES THEREOF IN REGULATING ANCILLARY GENE EXPRESSION

(71) Applicant: Zymergen Inc., Emeryville, CA (US)

(72) Inventors: Zach Serber, Sausalito, CA (US); Katherine G. Gora, Oakland, CA (US); Shawn P. Manchester, Oakland, CA (US); Peter Enyeart, Berkeley, CA (US); Alexander Shearer, Mountain View, CA (US)

(21) Appl. No.: 16/620,188

(22) PCT Filed: Jun. 7, 2018
(86) PCT No.: PCT/US2018/036472

§ 371 Date: Dec. 6, 2019

Related U.S. Application Data
(60) Provisional application No. 62/516,609, filed on Jun. 7, 2017.

(51) Int. Cl. C12N 15/77; C12P 13/08

(52) U.S. Cl. CPC C12N 15/77; C12P 13/08

(57) ABSTRACT

Provided are native promoters comprising polynucleotides isolated from Corynebacterium glutamicum, and mutant promoters derived therefrom, which may be used to regulate, i.e., either increase or decrease, on-pathway and/or off-pathway gene expression. Also provided are promoter ladders comprising a plurality of the promoters having incrementally increasing promoter activity. Also provided are host cells and recombinant vectors comprising the promoters, and methods of expressing ancillary genes of interest and producing biomolecules using the host cells.

### CROSS-REFERENCE TO RELATED APPLICATION

This application claims the benefit of priority under Section 119(e) of U.S. Provisional Application Ser. No. 62/516,609, entitled “PROMOTERS FROM CORYNEBACTERIUM GLUTAMICUM AND USES THEREOF IN REGULATING ANCILLARY GENE EXPRESSION,” filed Jun. 7, 2017, the disclosure of which is hereby incorporated by reference in the entirety and for all purposes.

### INCORPORATION BY REFERENCE OF THE SEQUENCE LISTING

The instant application contains a Sequence Listing which has been submitted electronically in ASCII format and is hereby incorporated by reference in its entirety. Said ASCII copy, created on Jun. 5, 2018, is named ZMG-004_PCT_SL.txt and is 645,695 bytes in size.

### BACKGROUND

### Field

The disclosure relates to native promoters comprising polynucleotides isolated from Corynebacterium glutamicum, and mutant promoters derived therefrom, host cells and recombinant vectors comprising the promoters, and methods of modifying the expression of ancillary target genes and producing biomolecules comprising culturing the host cells.

### Description of the Related Art

Strains of industrial important bacteria play a significant role in the production of biomolecules. For example, coryneform bacteria, in particular Corynebacterium glutamicum, can be cultured to produce biomolecules such as amino acids, organic acids, vitamins, nucleosides and nucleotides. Continuous efforts are being made to improve production processes. Said processes may be improved with respect to fermentation related measures such as, for example, stirring and oxygen supply, or the composition of nutrient media, such as, for example, sugar concentration during fermentation, nutrient feeding schedules, pH balance, metabolite removal, or the work-up into the product form, for example by means of ion exchange chromatography, or the intrinsic performance characteristics of the microorganism itself.

Performance characteristics can include, for example, yield, titer, productivity, by-product elimination, tolerance to process excursions, optimal growth temperature and growth rate. One way to improve performance of a microbial strain is to increase the expression of genes that control the production of a metabolite. Increasing expression of a gene can increase the activity of an enzyme that is encoded by that gene. Increasing enzyme activity can increase the rate of synthesis of the metabolic products made by the pathway to which that enzyme belongs. In some instances, increasing the rate of production of a metabolite can unbalance other cellular processes and inhibit growth of a microbial culture. Sometimes, down regulating activity is important to improve performance of a strain. For example, re-directing flux away from by-products can improve yield. Accordingly, fine-tuning of expression levels of the various components simultaneously within a metabolic pathway is often necessary.

Promoters regulate the rate at which genes are transcribed and can influence transcription in a variety of ways. Constitutive promoters, for example, direct the transcription of their associated genes at a constant rate regardless of the internal or external cellular conditions, while regulatable promoters increase or decrease the rate at which a gene is transcribed depending on the internal and/or the external cellular conditions, e.g. growth rate, temperature, responses to specific environmental chemicals, and the like. Promoters can be isolated from their normal cellular contexts and engineered to regulate the expression of virtually any gene, enabling the effective modification of cellular growth, product yield and/or other phenotypes of interest.

For the production of a target biomolecule, a promoter is typically functionally linked to a heterologous target gene that is a component of the biosynthetic pathway that makes the target biomolecule in the host cell. For example for production of lysine, a component of the lysine biosynthetic pathway (e.g., as defined in Kyoto Encyclopedia of Genes and Genomes (KEGG) pathway M00030) can be functionally linked to a heterologous promoter. The universe of such on-pathway components is finite and well-explored, and the potential for further optimization by modulating expression or activity of on-pathway target genes to optimize target biomolecule production is limited. However, the potential impact on the productivity and yield of such biomolecules afforded by operably linking heterologous promoters to one or more ancillary target genes, and thereby modulate expression of such target genes, in industrially important host strains has been largely unexplored. Thus, there remains a need in the art for methods and compositions to screen for, identify, and use ancillary target genes that can be modulated to increase or decrease expression or activity and thereby improve target biomolecule production.

### BRIEF SUMMARY

The present disclosure addresses these and other needs in the art. In brief, the present disclosure is directed to a host cell containing a promoter polynucleotide sequence functionally linked to at least one heterologous ancillary target gene, wherein the ancillary target gene is not a component of the biosynthetic pathway for producing the target biomolecule. The present disclosure provides methods for screening for, identifying, and using a promoter polynucleotide operably linked to a heterologous ancillary target gene to improve production of a target biomolecule.

In preferred embodiments, the promoter polynucleotide comprises a sequence selected from: SEQ ID NO:1, SEQ ID NO:2, SEQ ID NO:3, SEQ ID NO:4, SEQ ID NO:5, SEQ ID NO:6, SEQ ID NO:7, and SEQ ID NO:8. In some embodiments, the promoter polynucleotide consists of a sequence selected from:

SEQ ID NO:1, SEQ ID NO:5, or SEQ ID NO:7.

In some embodiments, the ancillary target gene is a gene that is classified under GOslim term GO:0003674; GO:0003677; GO:0008150; GO:0034641; or GO:0009058. Preferably, the ancillary target gene is a gene that is classified under, or under at least, 2, 3, 4, or 5 of the following GOslim terms GO:0003674; GO:0003677; GO:0008150; GO:0034641; or GO:0009058. In some embodiments, the ancillary target gene is selected from the genes of one or more, or all, of the following KEGG entries: M00010, M00002, M00007, M00580, or M00005.

In some embodiments, the ancillary target gene is not a component of a biosynthesis pathway comprising genes of one or more, or all, of the following KEGG entries: M00016; M00525; M00526; M00527; M00030; M00433 M00031; M00020; M00018; M00021; M00338; M00609; M00017; M00019; M00535; M00570; M00432; M00015; M00028; M00763; M00026; M00022; M00023; M00024; M00025; and M00040.

In one embodiment, the disclosure provides a host cell containing at least a first and a second promoter polynucleotide sequence, wherein the first promoter is functionally linked to a first heterologous target gene, wherein the first heterologous target gene is a component of a biosynthetic pathway for producing a target biomolecule, and the second promoter is functionally linked to a second heterologous ancillary target gene that is not a component of the biosynthetic pathway for producing the target biomolecule. In some embodiments, the first promoter can be a native promoter comprising polynucleotides isolated from Corynebacterium glutamicum, and/or a mutant promoter derived therefrom, which can each be encoded by short DNA sequences, ideally less than 100 base pairs, while the second promoter comprises a sequence selected from: SEQ ID NO:1, SEQ ID NO:2, SEQ ID NO:3, SEQ ID NO:4, SEQ ID NO:5, SEQ ID NO:6, SEQ ID NO:7, and SEQ ID NO:8. In some embodiments, both the first and the second promoter comprise a sequence selected from: SEQ ID NO:1, SEQ ID NO:2, SEQ ID NO:3, SEQ ID NO:4, SEQ ID NO:5, SEQ ID NO:6, SEQ ID NO:7, and SEQ ID NO:8. In some embodiments, the promoter polynucleotide consists of a sequence selected from: SEQ ID NO:1, SEQ ID NO:5, or SEQ ID NO:7.

One embodiment of the present disclosure relates to host cells comprising the first and/or second promoter polynucleotides described herein. One embodiment of the present disclosure relates to recombinant vectors comprising the first promoter polynucleotide and/or second promoter polynucleotide described herein. In some embodiments, the first promoter polynucleotide is functionally linked to a first on-pathway target gene. In some embodiments, the second promoter polynucleotide is functionally linked to a first or second ancillary target gene. One embodiment of the present disclosure relates to host cells comprising the combinations of promoter polynucleotides described herein. One embodiment of the present disclosure relates to recombinant vectors comprising the combinations of promoter polynucleotides described herein. In some embodiments, each promoter polynucleotide is functionally linked to a different target gene. Preferably, as described and demonstrated in more detail herein, the target genes are not part of the same metabolic pathway. In some embodiments, a first set of target genes are part of the same metabolic pathway and a second set of target genes are part of a different pathway. One embodiment of the present disclosure relates to host cells transformed with the recombinant vectors described herein.

One embodiment of the present disclosure relates to host cells comprising at least one promoter polynucleotide functionally linked to an ancillary target gene; wherein the promoter polynucleotide comprises a sequence selected from: SEQ ID NO:1, SEQ ID NO:2, SEQ ID NO:3, SEQ ID NO:4, SEQ ID NO:5, SEQ ID NO:6, SEQ ID NO:7, and SEQ ID NO:8; wherein when the promoter polynucleotide comprises a sequence selected from: SEQ ID NO:2, SEQ ID NO:3, SEQ ID NO:4, SEQ ID NO:6, or SEQ ID NO:8, the target gene is other than the promoter polynucleotide's endogenous gene. In some embodiments, the host cell comprises at least two promoter polynucleotides, wherein each promoter polynucleotide is functionally linked to a different target gene. One embodiment of the present disclosure relates to recombinant vectors comprising at least one promoter polynucleotide functionally linked to an ancillary target gene; wherein the promoter polynucleotide comprises a sequence selected from: SEQ ID NO:1, SEQ ID NO:2, SEQ ID NO:3, SEQ ID NO:4, SEQ ID NO:5, SEQ ID NO:6, SEQ ID NO:7, and SEQ ID NO:8; wherein when the promoter polynucleotide comprises a sequence selected from: SEQ ID NO:2, SEQ ID NO:3, SEQ ID NO:4, SEQ ID NO:6, or SEQ ID NO:8, the target gene is other than the promoter polynucleotide's endogenous gene.

In some embodiments, the recombinant vector comprises at least two promoter polynucleotides, wherein each promoter polynucleotide is functionally linked to a different target gene. Preferably, as described and demonstrated more fully herein, the target genes are not part of the same metabolic pathway. For example, one target gene can be an on-pathway target gene for production of a target biomolecule, and the second target gene can be an ancillary target gene.

One embodiment of the present disclosure relates to host cells transformed with the recombinant vectors described herein. In some cases, the transformed host cells comprise a combination of promoter polynucleotides functionally linked to a heterologous ancillary target gene or at least one heterologous ancillary target gene, wherein said combination of promoter polynucleotides comprises a promoter ladder. The individual promoter polynucleotides can be in different transformed host cells and operably linked to the same heterologous ancillary target gene sequence. In some embodiments, said combination of promoter polynucleotides comprises at least one first promoter polynucleotide, and at least one second promoter polynucleotide. In some embodiments, the first promoter polynucleotide is selected from the group consisting of SEQ ID NO:1, SEQ ID NO:5, and SEQ ID NO:7 and the second promoter polynucleotide is selected from the group consisting of: SEQ ID NO:2, SEQ ID NO:3, SEQ ID NO:4, SEQ ID NO:6, and SEQ ID NO:8 In some embodiments, said first and second promoter polynucleotide are in different host cells of a plurality of host cells and operably linked to the same heterologous ancillary target gene sequence. In some cases, the transformed host cells comprise a combination of promoter polynucleotides comprising a promoter ladder of two, three, four, five, six, seven, and/or eight different promoter polynucleotides. In some cases, said first, second, third, fourth, fifth, sixth, and/or seventh promoter polynucleotide are in different host cells of the plurality of transformed host cells and operably linked to the same heterologous ancillary target gene sequence.

In some cases, the transformed host cells comprising the combination of promoter polynucleotides functionally linked to a heterologous ancillary target gene or at least one heterologous ancillary target gene, wherein said combination of promoter polynucleotides comprises a promoter ladder, further comprises a promoter polynucleotide operably linked to an on-pathway, a shell 1, and/or a shell 2 heterologous target gene. In some cases, each of the transformed host cells, substantially all of the transformed host cells, or a majority of the transformed host cells comprises a promoter polynucleotide operably linked to an on-pathway, a shell 1, and/or a shell 2 heterologous target gene.

One embodiment of the present disclosure relates to methods of modifying the expression of one or more ancillary target genes, comprising culturing a host cell described herein, wherein the modification of each ancillary target gene is independently selected from: up-regulating and down-regulating. Preferably, the ancillary target gene does not code for one or more polypeptides or proteins of a biosynthetic pathway of biomolecules such as an amino acid, organic acid, nucleic acid, protein, or polymer. For example, in some embodiments, the ancillary target gene may code for one or more polypeptides or proteins of the biosynthetic pathway of a transcription factor, a signaling molecule, a component of the citric acid cycle, or a component of glycolysis.

Another embodiment of the present disclosure relates to methods of producing a biomolecule comprising culturing a host cell described herein, under conditions suitable for producing the biomolecule. In some embodiments the ancillary target gene directly or indirectly enhances the biosynthesis of a biomolecule selected from: amino acids, organic acids, flavors and fragrances, biofuels, proteins and enzymes, polymers/monomers and other biomaterials, lipids, nucleic acids, small molecule therapeutics, protein or peptide therapeutics, fine chemicals, and nutraceuticals. In preferred embodiments, the biomolecule is an L-amino acid. In specific embodiments, the L-amino acid is lysine.

In some embodiments, the host cell belongs to the genus Corynebacterium. In some embodiments, the host cell is Corynebacterium glutamicum.

### BRIEF DESCRIPTION OF THE DRAWINGS

FIG. 1 presents a diagram of the genetic and biochemical pathway for the biosynthesis of the amino acid L-lysine. Genes that divert intermediates in the biosynthetic pathway (e.g., pck, odx, icd, and hom) are underlined.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

As used herein, the term “recombinant nucleic acid molecule” refers to a recombinant DNA molecule or a recombinant RNA molecule. A recombinant nucleic acid molecule is any nucleic acid molecule containing joined nucleic acid molecules from different original sources and not naturally attached together. Recombinant RNA molecules include RNA molecules transcribed from recombinant DNA molecules. In particular, a recombinant nucleic acid molecule includes a nucleic acid molecule comprising a promoter of SEQ ID NOs:1 to 8 functionally linked to a heterologous target gene.

As used herein, the term “heterologous target gene” refers to any gene or coding sequence that is not controlled in its natural state (e.g., within a non-genetically modified cell) by the promoter to which it is operably linked in a particular genome. As provided herein, all target genes functionally linked to non-naturally occurring promoters are considered “heterologous target genes”. More specifically, as promoter polynucleotide sequences of SEQ ID NOs:1, 5, and 7 do not occur in nature, all functionally linked target gene sequences are “heterologous target gene” sequences. Similarly, all, e.g., naturally occurring, target genes in a host cell that are functionally linked with a promoter that is naturally occurring in the host cell but is not normally functionally linked to said target gene in a wild-type organism are “heterologous target genes.” As used herein, a heterologous target gene can include one or more target genes that are part of an operon. That is, the endogenous promoter of an operon is replaced with a promoter polynucleotide sequence having a nucleic sequence of SEQ ID NOs:1 to 8. As used herein, the term “promoter polynucleotide sequence” refers to nucleic acids having a sequence as recited in the associated SEQ ID NO.

A “metabolic pathway” or “biosynthetic pathway” is a series of substrate to product conversion reactions, each of which is catalysed by a gene product (e.g., an enzyme), wherein the product of one conversion reaction acts as the substrate for the next conversion reaction and which includes the conversion reactions from a feedstock to a target biomolecule. In some embodiments, the metabolic pathway is a pathway module as defined in the Kyoto Encyclopedia of Genes and Genomes KEGG database. As used herein, reference to the KEGG database, including maps and pathway modules therein, refers to the database as it is publicly available on the priority date of the present application.

An “on-pathway” heterologous target gene is a heterologous target gene that encodes a gene product (e.g., an enzyme or a component of a multi-enzyme complex) that is in the metabolic pathway by which the target biomolecule is produced in the organism in which it is present. Conventionally, the genes targeted for modification are those genes that are judged to be “on-pathway,” i.e., the genes for the metabolic enzymes known to be part of, or branching into or off of, the biosynthetic pathway for the molecule of interest (Keasling, JD. “Manufacturing molecules through metabolic engineering.” Science, 2010). Methods such as flux balance analysis (“FBA”) (Segre et al, “Analysis of optimality in natural and perturbed metabolic networks.” PNAS, 2002) are known that can automate the discovery of such genes.

An “ancillary” or “off-pathway” heterologous target gene, or heterologous target gene that is “not a component of a biosynthetic pathway for production of a target molecule” and the like is a heterologous target gene that does not encode a gene product (e.g., an enzyme or a component of a multi-enzyme complex) that is in the metabolic pathway by which the target biomolecule is produced in the organism in which it is present.

For example, an ancillary or off-pathway heterologous target gene for production of the target biomolecule L-lysine is a gene that is not disclosed in KEGG pathway module M00016, M00030, M00031, M00433, M00525, M00526, or M00527, or preferably all thereof. As another example, an ancillary or off-pathway heterologous target gene for production of the target biomolecule serine is a gene that is not disclosed in KEGG pathway module M00020. As another example, an ancillary or off-pathway heterologous target gene for production of the target biomolecule threonine is a gene that is not disclosed in KEGG pathway module M00018. As another example, an ancillary or off-pathway heterologous target gene for production of the target biomolecule cysteine is a gene that is not disclosed in KEGG pathway module M00021, M00338, or M00609, or preferably all thereof.

As yet another example, an ancillary or off-pathway heterologous target gene for production of the target biomolecule valine and/or isoleucine is a gene that is not disclosed in KEGG pathway module M00019. As yet another example, an ancillary off-pathway heterologous target gene for production of the target biomolecule isoleucine is a gene that is not disclosed in KEGG pathway module M00535, or M00570, or preferably all thereof. As yet another example, an ancillary or off-pathway heterologous target gene for production of the target biomolecule leucine is a gene that is not disclosed in KEGG pathway module M00432.

As yet another example, an ancillary or off-pathway heterologous target gene for production of the target biomolecule proline is a gene that is not disclosed in KEGG pathway module M00015. As yet another example, an ancillary or off-pathway heterologous target gene for production of the target biomolecule ornithine is a gene that is not disclosed in KEGG pathway module M00028, M00763, or preferably all thereof. As yet another example, an ancillary or off-pathway heterologous target gene for production of the target biomolecule histidine is a gene that is not disclosed in KEGG pathway module M00026.

As yet another example, aromatic amino acids such as tryptophan, tyrosine, and phenylalanine are produced via the shikimate pathway. Thus, an ancillary or off-pathway heterologous target gene for production of the target biomolecule shikimate or an amino acid that is a biosynthetic product of the shikimate pathway (e.g., one or more of the target biomolecules tryptophan, tyrosine, or phenylalanine) is a gene that is not disclosed in KEGG pathway module M00022. As yet another example, an ancillary or off-pathway heterologous target gene for production of the target biomolecule tryptophan is a gene that is not disclosed in KEGG pathway module M00022. As yet another example, an ancillary or off-pathway heterologous target gene for production of the target biomolecule phenylalanine is a gene that is not disclosed in KEGG pathway module M00024. As yet another example, an ancillary or off-pathway heterologous target gene for production of the target biomolecule tyrosine is a gene that is not disclosed in KEGG pathway module M00025, M00040, or the combination thereof.

As yet another example, in some embodiments, in the context of producing an L-lysine target biomolecule, a heterologous target gene that is a component of the biosynthetic pathway that produces L-lysine is one of the following genes, or an endogenous functional ortholog thereof in the organism in which it is present, asd, ask, aspB, cg0931, dapA, dapB, dapD, dapE, dapF, ddh, fbp, hom, icd, lysA, lysE, odx, pck, pgi, ppc, ptsG, pyc, tkt, or zwf. Accordingly, in the context of producing an L-lysine target biomolecule, an ancillary or off-pathway heterologous target gene is a gene that is not one of the following genes, or an endogenous functional ortholog thereof in the organism in which it is present, asd, ask, aspB, cg0931, dapA, dapB, dapD, dapE, dapF, ddh, fbp, hom, icd, lysA, lysE, odx, pck, pgi, ppc, ptsG, pyc, tkt, or zwf.

In some embodiments, target genes are divided into priority levels, called “shells” and promoter polynucleotides are operably linked to one or more heterologous target genes of a shell, wherein the shell is comprised genes that are indirectly involved in target molecule production. As used herein, “shell 1” genes are genes that encode biosynthetic enzymes directly involved in a selected metabolic pathway. “Shell 2” genes include genes encoding for non-shell 1 enzymes or other proteins within the biosynthetic pathway responsible for product diversion or feedback signaling. “Shell 3” genes include regulatory genes responsible for modulating expression of the biosynthetic pathway or for regulating carbon flux within the host cell. “Shell 4” genes are the genes of a target organism that are not assigned to any one of shells 1-3. Example 5 describes allocation of genes in C. glutamicum into shells for systematic genome-wide perturbation of lysine production.

In some cases, an ancillary heterologous target gene is a “shell 2,” “shell 3,” and/or “shell 4” heterologous target gene for production of a target molecule. In some cases, an ancillary heterologous target gene is a “shell 3” and/or “shell 4” heterologous target gene for production of a target molecule. In some cases, the ancillary heterologous target gene is a “shell 3” heterologous target gene for production of a target molecule. In some cases, the ancillary heterologous target gene is a “shell 4” heterologous target gene for production of a target molecule. In some cases, the ancillary heterologous target gene is a “shell 2” heterologous target gene for production of a target molecule.

Exemplary target genes and their shell designation in the context of lysine production in C. glutamicum are provided in Table 10 below.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

### Polynucleotides Having Promoter Activity

Native C. glutamicum promoters were identified that satisfy both of the following criteria: 1) represent a ladder of constitutive promoters, i.e., a plurality of promoters with incrementally increasing levels of promoter activity; and 2) encoded by short DNA sequences, ideally less than 100 base pairs. A published data set describing global gene expression levels in C. glutamicum ATCC 13032 (Lee et al., Biotechnol Lett (2013) 35:709-717) was examined to identify genes that were constitutively expressed across different growth conditions. Genes whose expression level remained constant (defined as a ratio of expression between 0.33 and 3) across two growth conditions, namely chemostat growth in minimal media with and without the addition of hydrogen peroxide satisfied the first criterion. A published data set describing the C. glutamicum ATCC 13032 transcriptome (Pfeifer-Sancar et al., BMC Genomics 2013, 14:888) was examined to find genes with compact promoters, i.e. those consisting of the 60 base pair core promoter region and a 5′ untranslated region between 26 and 40 base pairs in length. The two data sets were cross-referenced to identify promoters that satisfied both criteria. The following five wild-type promoters were identified (Table 1).

TABLE 1
Promoters of C. glutamicum Having Increasing Levels of Expression
and Constituent Expression Under Different Growth Conditions

| Strain | SEQ ID NO | Mean Activity |
|---|---|---|
| Pcg1860-eyfp | 2 | 89243 |
| Pcg0007-eyfp | 3 | 44527 |
| Pcg0755-eyfp | 4 | 43592 |
| Pcg3381-eyfp | 6 | 4723 |
| Pcg3121-eyfp | 8 | 98 |

The wild-type promoters Pcg1860, and Pcg3121 are not described in the literature. The wild-type promoter Pcg0007-gyrB is also not described in the literature, however, Neumann and Quiñones, (J Basic Microbiol. 1997; 37(1):53-69) describes regulation of gyrB gene expression in E. coli. The wild type promoter Pcg0755 is a known part of the methionine biosynthesis pathway (Suda et al., Appl Microbiol Biotechnol (2008) 81:505-513; and Rey et al., Journal of Biotechnology 103 (2003) 51-65). The wild-type promoter Pcg3381 is a tatA homolog. The tatA pathway in Corynebacterium is described by Kikuchi et al., Applied and Environmental Microbiology, November 2006, p. 7183-7192. The strong constitutive promoter Pcg0007 was chosen for mutagenesis. Four out of six positions in the predicted ˜10 element (TAAGAT) of Pcg0007 were randomized to generate both stronger and attenuated promoter variants (SEQ ID NOs 1, 5, and 7).

Following the identification of promoters comprising SEQ ID NOs: 1-8, the present inventors determined that one or more such promoters can be functionally linked to one or more heterologous target genes of a biosynthetic pathway to increase the production of a target biomolecule produced by that biosynthetic pathway in a host cell. The identification and characterization of promoters of SEQ ID NOs: 1-8, and their use in upregaulting and/or downregulating expression of one or more on-pathway heterologous target genes to produce a target biomolecule are further described in PCT Appl. No. PCT/US16/65464, filed Dec. 7, 2016, the contents of which are hereby incorporated by reference in the entirety and for all purposes, including but not limited to the promoters of SEQ ID NO:1-8; vectors, expression cassettes, and host cells comprising said promoters, whether or not operably linked to a heterologous target gene, and methods and compositions for production of target biomolecules (e.g., using a promoter of SEQ ID NO:1-8).

Additionally, the present inventors surprisingly discovered that functionally linking one or more such promoters to one or more ancillary or off-pathway heterologous target genes can be used to increase production of the target biomolecule or further increase production of the target biomolecule.

For example, in some embodiments, functionally linking one or more such promoters to one or more ancillary heterologous target genes can be used to increase production of the target biomolecule in a strain background that does not have a promoter functionally linked to a heterologous target gene that is a component of the biosynthetic pathway that produces the target biomolecule. Additionally, in some embodiments, functionally linking one or more such promoters to one or more ancillary heterologous target genes can be used to increase production of the target biomolecule in a strain background that also comprises one or more promoters functionally linked to one or more heterologous target genes that are components of the biosynthetic pathway that produces the target biomolecule.

In some cases, the one or more promoters functionally linked to one or more heterologous target genes that are components of the biosynthetic pathway for production of a target biomolecule can be selected from SEQ ID NOs:1-8, SEQ ID NOs: 1, 5, and 7, and other promoters known in the art. Similarly, in some cases, the one or more promoters functionally linked to one or more ancillary heterologous target genes that are not components of the biosynthetic pathway for production of a target biomolecule can be selected from SEQ ID NOs:1-8, SEQ ID NOs: 1, 5, and 7, and other promoters known in the art.

Accordingly, one embodiment of the present disclosure relates to native promoters comprising polynucleotides isolated from C. glutamicum, and mutant promoters derived therefrom that together represent a ladder of constitutive promoters with incrementally increasing levels of promoter activity, wherein one or more of the ladder of promoters is functionally linked to a heterologous ancillary target gene for production of a target biomolecule. In some embodiments, a C. glutamicum promoter can be encoded by a short DNA sequence. In some embodiments a C. glutamicum promoter can be encoded by a DNA sequence of less than 100 base pairs. The promoters can be used in any strain background, including strains that also include a promoter functionally linked to a heterologous target gene that is in a biosynthetic pathway for production of a target biomolecule.

One embodiment of the present disclosure relates to a promoter polynucleotide comprising a sequence selected from: SEQ ID NO:1 (Pcg0007_lib_39), SEQ ID NO:2 (Pcg1860), SEQ ID NO:3 (Pcg0007), SEQ ID NO:4 (Pcg0755), SEQ ID NO:5 (Pcg0007_lib_265), SEQ ID NO:6 (Pcg3381), SEQ ID NO:7 (Pcg0007_lib_119), or SEQ ID NO:8 (Pcg3121). In another embodiment, the present specification provides for, and includes, a promoter polynucleotide comprising of SEQ ID NO:1 functionally linked to at least one heterologous ancillary target gene. In an embodiment, the present specification provides for, and includes, a promoter polynucleotide of SEQ ID NO:2 functionally linked to at least one heterologous ancillary target gene. In another embodiment, the present specification provides for, and includes, a promoter polynucleotide of SEQ ID NO:3 functionally linked to at least one heterologous ancillary target gene. In another embodiment, the present specification provides for, and includes, a promoter polynucleotide of SEQ ID NO:4 functionally linked to at least one heterologous ancillary target gene. In another embodiment, the present specification provides for, and includes, a promoter polynucleotide of SEQ ID NO:5 functionally linked to at least one heterologous ancillary target gene. In another embodiment, the present specification provides for, and includes, a promoter polynucleotide comprising of SEQ ID NO:5 functionally linked to at least one heterologous ancillary target gene. In another embodiment, the present specification provides for, and includes, a promoter polynucleotide of SEQ ID NO:7 functionally linked to at least one heterologous ancillary target gene. In another embodiment, the present specification provides for, and includes, a promoter polynucleotide of SEQ ID NO:8 functionally linked to at least one heterologous ancillary target gene.

As used herein, a “promoter cassette” refers to the polynucleotide sequences comprising a promoter polynucleotide of SEQ ID NOs:1 to 8 functionally linked to at least one heterologous ancillary target gene. In certain embodiments of the present disclosure, a “promoter cassette” may further include one or more of a linker polynucleotide, a transcription terminator following the ancillary target gene, a ribosome binding site upstream of the start codon of the ancillary target gene, and combinations of each.

One embodiment of the present disclosure relates to a promoter polynucleotide consisting of a sequence selected from: SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In an embodiment, the present specification provides for, and includes a promoter polynucleotide sequence of SEQ ID NO:1. In an embodiment, the present specification provides for, and includes a promoter polynucleotide sequence of SEQ ID NO:5. In an embodiment, the present specification provides for, and includes a promoter polynucleotide sequence of SEQ ID NO:7. As used herein, a promoter cassette may be described by reference to the promoter name followed by the name of the heterologous target gene that is functionally linked to it. For example, the promoter of SEQ ID NO: 2, entitled Pcg1860, functionally linked to the gene zwf encoding the off-pathway glucose-6-phosphate 1-dehydrogenase gene is referenced as Pcg1860-zwf. Similarly, Pcg0007_39-lysA is the 0007_39 promoter of SEQ ID NO:1 functionally linked to target gene lysA encoding the polypeptide diaminopimelate decarboxylase.

One embodiment of the present disclosure relates to combinations of the promoter polynucleotides described herein. In this context the term “combinations of promoter polynucleotides” refers to two or more polynucleotides that may be present as separate isolated sequences, as components of separate polynucleotide molecules, or as components of the same polynucleotide molecule, and combinations thereof. Examples of polynucleotide molecules include chromosomes and plasmids.

The disclosure also relates to an isolated promoter polynucleotide, which essentially consists of a polynucleotide having the nucleotide sequence depicted in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In an embodiment, the present specification provides for, and includes an isolated promoter polynucleotide of SEQ ID NO:1. In an embodiment, the present specification provides for, and includes an isolated promoter polynucleotide of SEQ ID NO:5. In an embodiment, the present specification provides for, and includes an isolated promoter polynucleotide of SEQ ID NO:7.

The term “essentially” in this context means that a polynucleotide of no more than 1,000, no more than 800, no more than 700, no more than 600, no more than 500 or no more than 400 nucleotides in length; and a polynucleotide of no more than 15,000, no more than 10,000, no more than 7,500, no more than 5,000, no more than 2,500, no more than 1,000, no more than 800, no more than 700, no more than 600, no more than 500, or no more than 400 nucleotides in length have been added to the 5′ end and 3′ end, respectively, of the polynucleotides of SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

Any useful combination of the features from the preceding two lists of polynucleotides added to the 5′ end and 3′ end, respectively, of the polynucleotides of SEQ ID NO:1, SEQ ID NO:2, SEQ ID NO:3, SEQ ID NO:4, SEQ ID NO:5, SEQ ID NO:6, SEQ ID NO:7, or SEQ ID NO:8, is in accordance with the invention here. “Useful combination” means, for example, a combination of features which results in an efficient recombination being carried out. The use of additions of the same length flanking a DNA region to be replaced facilitates the transfer of the region by homologous recombination in the experimental procedure. Relatively long flanking homologous regions are advantageous for efficient recombination between circular DNA molecules but cloning of the replacement vector is made more difficult with increasing length of the flanks (Wang et al., Molecular Biotechnology, 432:43-53 (2006)). The specification provides for, and includes, homologous regions flanking a promoter polynucleotide sequence of SEQ ID NOs:1 to 8 functionally linked to at least one heterologous ancillary target gene (e.g., the “promoter cassette”) to direct homologous recombination and replacement of a target gene sequence. In an embodiment, the homologous regions are direct repeat regions. In an embodiment, the homologous regions comprises between 500 base pairs (bp) and 5000 bp each of the target gene sequence flanking the promoter cassette. In an embodiment, the homologous regions comprises at least 500 bp each of the target gene sequence flanking the promoter cassette. In an embodiment, the homologous regions comprises at least 1000 bp (1 Kb) each of the target gene sequence flanking the promoter cassette. In an embodiment, the homologous regions comprises at least 2 Kb each of the target gene sequence flanking the promoter cassette. In an embodiment, the homologous regions comprises at least 5 Kb each of the target gene sequence flanking the promoter cassette.

The disclosure furthermore relates to an isolated promoter polynucleotide, which consists of the nucleotide sequence depicted in SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In an embodiment, the isolated promoter polynucleotide consists of the polynucleotide sequence of SEQ ID NO:1. In an embodiment, the isolate promoter polynucleotide consists of the polynucleotide sequence of SEQ ID NO:5. In an embodiment, the isolate promoter polynucleotide consists of the polynucleotide sequence of SEQ ID NO:7.

Details regarding the biochemistry and chemical structure of polynucleotides as present in living things such as microorganisms, for example, can be found inter alia in the text book “Biochemie” [Biochemistry] by Berg et al. (Spektrum Akademischer Verlag Heidelberg Berlin, Germany, 2003; ISBN 3-8274-1303-6).

Polynucleotides consisting of deoxyribonucleotide monomers containing the nucleobases or bases adenine (A), guanine (G), cytosine (C) and thymine (T) are referred to as deoxyribo-polynucleotides or deoxyribonucleic acid (DNA). Polynucleotides consisting of ribonucleotide monomers containing the nucleobases or bases adenine (A), guanine (G), cytosine (C) and uracil (U) are referred to as ribopolynucleotides or ribonucleic acid (RNA). The monomers in said polynucleotides are covalently linked to one another by a 3′,5′-phosphodiester bond.

A “promoter polynucleotide” or a “promoter” or a “polynucleotide having promoter activity” means a polynucleotide, preferably deoxyribopolynucleotide, or a nucleic acid, preferably deoxyribonucleic acid (DNA), which when functionally linked to a polynucleotide to be transcribed determines the point and frequency of initiation of transcription of the coding polynucleotide, thereby enabling the strength of expression of the controlled polynucleotide to be influenced. The term “promoter ladder” as used herein refers to a plurality of promoters with incrementally increasing levels of promoter activity. The term “promoter activity” as used herein refers to the ability of the promoter to initiate transcription of an polynucleotide sequence into mRNA. Methods of assessing promoter activity are well known to those of skill in the art and include, for example the methods described in Example 2 of PCT/US16/65464. The term “constitutive promoter” as used herein refers to a promoter that directs the transcription of its associated gene at a constant rate regardless of the internal or external cellular conditions. In some cases, the promoters of the promoter ladder exhibit a range of promoter strengths in response to a stimuli (e.g., in response to induction with a chemical agent, heat, cold, stress, phosphate starvation, etc.). In some cases, the promoters of the promoter ladder exhibit a range of constitutive promoter strengths.

Owing to the double-stranded structure of DNA, the strand complementary to the strand in SEQ ID NO:1, SEQ ID NO:2, SEQ ID NO:3, SEQ ID NO:4, SEQ ID NO:5, SEQ ID NO:6, SEQ ID NO:7, or SEQ ID NO:8 of the sequence listing is likewise a subject of the invention.

### Kits

One embodiment of the present disclosure relates to kits comprising a first promoter polynucleotide comprising a sequence selected from: SEQ ID NO:1, SEQ ID NO:5, and SEQ ID NO:7, and a suitable storage means for the polynucleotide. In some embodiments, the first promoter polynucleotide consists of a sequence selected from: SEQ ID NO:1, SEQ ID NO:5, and SEQ ID NO:7. In some embodiments, the kits comprise combinations of promoter polynucleotides comprising at least two first promoter polynucleotides described herein. In some embodiments, the kits comprise combinations of promoter polynucleotides comprising at least one first promoter polynucleotide described herein, and at least one second promoter polynucleotide comprising a sequence selected from: SEQ ID NO:2, SEQ ID NO:3, SEQ ID NO:4, SEQ ID NO:6, and SEQ ID NO:8. In some embodiments, the kits comprise combinations of promoter polynucleotides comprising at least one first promoter polynucleotide described herein, and at least one second promoter polynucleotide consisting of a sequence selected from: SEQ ID NO:2, SEQ ID NO:3, SEQ ID NO:4, SEQ ID NO:6, and SEQ ID NO:8.

### Target Genes

One embodiment of the present disclosure relates to methods of modulating the expression of a heterologous target gene, comprising culturing a host cell transformed with a recombinant vector comprising a promoter polynucleotide as described herein. Heterologous target genes are polynucleotides the expression of which are controlled by the promoters described herein. The heterologous target genes may be coding polynucleotides which code for one or more polypeptide(s) or non-coding polynucleotides such as non-coding RNAs. A polynucleotide coding for a protein/polypeptide essentially consists of a start codon selected from the group consisting of ATG, GTG and TTG, preferably ATG or GTG, particularly preferably ATG, a protein-encoding sequence and one or more stop codon(s) selected from the group consisting of TAA, TAG and TGA. The heterologous target genes can be “on-pathway,” or “off-pathway,” or a combination thereof.

“Transcription” means the process by which a complementary RNA molecule is produced starting from a DNA template. This process involves proteins such as RNA polymerase, “sigma factors” and transcriptional regulatory proteins. Where the target gene is a coding polynucleotide, the synthesized RNA (messenger RNA, mRNA) then serves as a template in the process of translation which subsequently yields the polypeptide or protein.

“Functionally linked” means in this context the sequential arrangement of the promoter polynucleotide according to the disclosure with a further oligo- or polynucleotide, resulting in transcription of said further polynucleotide to produce a sense RNA transcript.

If the further polynucleotide is a target gene which codes for a polypeptide/protein and consists of the coding region for a polypeptide, starting with a start codon, including the stop codon and, where appropriate, including a transcription termination sequence, “functionally linked” then means the sequential arrangement of the promoter polynucleotide according to the invention with the target gene, resulting in transcription of said target gene and translation of the synthesized RNA.

If the target gene codes for a plurality of proteins/polypeptides, each gene may be preceded by a ribosome-binding site. Where appropriate, a termination sequence is located downstream of the last gene.

The target gene preferably codes for one or more polypeptides or proteins of the biosynthetic pathway of biomolecules, preferably selected from the group of proteinogenic amino acids, non-proteinogenic amino acids, vitamins, nucleosides, nucleotides and organic acids. The target gene preferably consists of one or more of the one-pathway and/or off-pathway target genes listed in Table 1 of EP 1 108 790 A2 which is hereby incorporated by reference.

The present specification provides for, and includes, recombinant nucleic acid molecules comprising a promoter polynucleotide sequence selected from the group consisting of SEQ ID NOs:1 to 8 functionally linked to any one of the heterologous target genes identifiable in the Kyoto Encyclopedia of Genes and Genomes (KEGG) as genes involved in metabolic and biosynthetic pathways. The KEGG database is available on the internet at genome.jp/kegg.

In preferred embodiments, the target biomolecule is an amino acid, a protein, or a carbohydrate polymer, and one or more of the promoter polynucleotide sequences of SEQ ID NOs:1 to 8 are functionally linked to one or more ancillary target genes of the citric acid cycle. In some cases, the ancillary target genes are selected from the genes in KEGG pathway M00010. In one embodiment, the target biomolecule is an amino acid, a protein, or a carbohydrate polymer and one or more of the promoter polynucleotide sequences of SEQ ID NOs:1 to 8 are functionally linked to one or more ancillary target genes of the glycolysis pathway. In some cases, the ancillary target genes are selected from the genes in KEGG pathway M00002. In one embodiment, the target biomolecule is an amino acid, a protein, or a carbohydrate polymer and one or more of the promoter polynucleotide sequences of SEQ ID NOs:1 to 8 are functionally linked to one or more ancillary target genes of the pentose phosphate pathway. In some cases, the ancillary target genes are selected from the genes in KEGG pathway M00007, or M00580, or the combination thereof.

In one embodiment, the target biomolecule is an amino acid, a protein, or a carbohydrate polymer and one or more of the promoter polynucleotide sequences of SEQ ID NOs:1 to 8 are functionally linked to one or more ancillary target genes of the PRPP biosynthesis pathway. In some cases, the ancillary target genes are selected from the genes in KEGG pathway M00005. In some cases, the target biomolecule is a specific amino acid or a set of amino acids, and one or more of the promoter polynucleotide sequences of SEQ ID NOs:1 to 8 are functionally linked to one or more ancillary target genes selected from a metabolic pathway for production of a different amino acid or set of amino acids.

In an embodiment, the promoter polynucleotide sequences of SEQ ID NOs:1 to 8 are functionally linked to one or more on-pathway target genes of the lysine biosynthesis pathway as represented in KEGG map number 00300. In an embodiment, the one or more on-pathway target genes are selected from the Lysine succinyl-DAP biosynthesis pathway, M00016. In an embodiment, the one or more on-pathway target genes are selected from the lysine acetyl-DAP biosynthesis pathway, M00525. In an embodiment, the one or more on-pathway target genes are selected from the lysine DAP dehydrogenase biosynthesis pathway, M00526. In an embodiment, the one or more on-pathway target genes are selected from the lysine DAP aminotransferase biosynthesis pathway, M00527. In an embodiment, the one or more on-pathway target genes are selected from the AAA pathway biosynthesis pathway, M00030. In an embodiment, the one or more on-pathway target genes are selected from the lysine biosynthesis pathway from 2-oxoglutarate, M00433 or the lysine biosynthesis pathway mediated by LysW, M00031.

The present disclosure provides for, and includes, the promoter polynucleotide sequences of SEQ ID NOs:1 to 8 are functionally linked to one or more on-pathway target genes of the serine biosynthesis pathway comprising genes of entry M00020. In an embodiment, the promoter polynucleotide sequences of SEQ ID NOs:1 to 8 are functionally linked to one or more on-pathway target genes of the threonine biosynthesis pathway comprising genes of KEGG entry M00018. In an embodiment, the promoter polynucleotide sequences of SEQ ID NOs:1 to 8 are functionally linked to one or more on-pathway target genes of the cysteine biosynthesis pathway comprising genes of KEGG entry M00021. In an embodiment, the promoter polynucleotide sequences of SEQ ID NOs:1 to 8 are functionally linked to one or more on-pathway target genes of the cysteine biosynthesis pathway comprising genes of KEGG entry M00338. In an embodiment, the promoter polynucleotide sequences of SEQ ID NOs:1 to 8 are functionally linked to one or more on-pathway target genes of the cysteine biosynthesis pathway comprising genes of KEGG entry M00609. In an embodiment, the promoter polynucleotide sequences of SEQ ID NOs:1 to 8 are functionally linked to one or more on-pathway target genes of the methionine biosynthesis pathway comprising genes of KEGG entry M00017. In an embodiment, the promoter polynucleotide sequences of SEQ ID NOs:1 to 8 are functionally linked to one or more on-pathway target genes of the valine/isoleucine biosynthesis pathway comprising genes of KEGG entry M00019. In an embodiment, the promoter polynucleotide sequences of SEQ ID NOs:1 to 8 are functionally linked to one or more on-pathway target genes of the isoleucine biosynthesis pathway comprising genes of KEGG entry M00535. In an embodiment, the promoter polynucleotide sequences of SEQ ID NOs:1 to 8 are functionally linked to one or more on-pathway target genes of the isoleucine biosynthesis pathway comprising genes of KEGG entry M00570. In an embodiment, the promoter polynucleotide sequences of SEQ ID NOs:1 to 8 are functionally linked to one or more on-pathway target genes of the leucine biosynthesis pathway comprising genes of KEGG entry M00432. In an embodiment, the promoter polynucleotide sequences of SEQ ID NOs:1 to 8 are functionally linked to one or more on-pathway target genes of the proline biosynthesis pathway comprising genes of KEGG entry M00015. In an embodiment, the promoter polynucleotide sequences of SEQ ID NOs:1 to 8 are functionally linked to one or more on-pathway target genes of the ornithine biosynthesis pathway comprising genes of KEGG entry M00028. In an embodiment, the promoter polynucleotide sequences of SEQ ID NOs:1 to 8 are functionally linked to one or more on-pathway target genes of the ornithine biosynthesis pathway comprising genes of KEGG entry M00763. In an embodiment, the promoter polynucleotide sequences of SEQ ID NOs:1 to 8 are functionally linked to one or more on-pathway target genes of the histidine biosynthesis pathway comprising genes of KEGG entry M00026. In an embodiment, the promoter polynucleotide sequences of SEQ ID NOs:1 to 8 are functionally linked to one or more on-pathway target genes of the shikimate biosynthesis pathway comprising genes of KEGG entry M00022. In an embodiment, the promoter polynucleotide sequences of SEQ ID NOs:1 to 8 are functionally linked to one or more on-pathway target genes of the tryptophan biosynthesis pathway comprising genes of entry M00023. In an embodiment, the promoter polynucleotide sequences of SEQ ID NOs:1 to 8 are functionally linked to one or more on-pathway target genes of the phenylalanine biosynthesis pathway comprising genes of KEGG entry M00024. In an embodiment, the promoter polynucleotide sequences of SEQ ID NOs:1 to 8 are functionally linked to one or more on-pathway target genes of the tyrosine biosynthesis pathway comprising genes of KEGG entry M00025. In an embodiment, the promoter polynucleotide sequences of SEQ ID NOs:1 to 8 are functionally linked to one or more on-pathway target genes of the tyrosine biosynthesis pathway comprising genes of KEGG entry M00040.

In a preferred embodiment, one or more of the promoter polynucleotide sequences of SEQ ID NOs:1 to 8 are functionally linked to one or more on-pathway target genes described herein and one or more promoter polynucleotide sequences of SEQ ID NOs:1 to 8 are functionally linked to one or more ancillary target genes described herein, e.g., in a host cell, a genome of a host cell, an expression cassette, and/or a polynucleotide vector. In another embodiment, one or more of the promoter polynucleotide sequences of SEQ ID NOs:1 to 8 are functionally linked to one or more on-pathway target genes described herein and one or more other promoter polynucleotide sequences are functionally linked to one or more ancillary target genes described herein, e.g., in a host cell, a genome of a host cell, an expression cassette, and/or a polynucleotide vector. In yet another embodiment, one or more of the promoter polynucleotide sequences of SEQ ID NOs:1 to 8 are functionally linked to one or more ancillary target genes described herein and one or more other promoter polynucleotide sequences are functionally linked to one or more on-pathway target genes described herein, e.g., in a host cell, a genome of a host cell, an expression cassette, and/or a polynucleotide vector.

The present disclosure provides for, and includes, the promoter polynucleotide sequences of SEQ ID NOs:1, 5 or 7 are functionally linked to one or more target genes of the serine biosynthesis pathway comprising genes of entry M00020. In an embodiment, the promoter polynucleotide sequences of SEQ ID NOs:1, 5 or 7 are functionally linked to one or more target genes of the threonine biosynthesis pathway comprising genes of KEGG entry M00018. In an embodiment, the promoter polynucleotide sequences of SEQ ID NOs:1, 5 or 7 are functionally linked to one or more target genes of the cysteine biosynthesis pathway comprising genes of KEGG entry M00021. In an embodiment, the promoter polynucleotide sequences of SEQ ID NOs:1, 5 or 7 are functionally linked to one or more target genes of the cysteine biosynthesis pathway comprising genes of KEGG entry M00338. In an embodiment, the promoter polynucleotide sequences of SEQ ID NOs:1, 5 or 7 are functionally linked to one or more target genes of the cysteine biosynthesis pathway comprising genes of KEGG entry M00609. In an embodiment, the promoter polynucleotide sequences of SEQ ID NOs:1, 5 or 7 are functionally linked to one or more target genes of the methionine biosynthesis pathway comprising genes of KEGG entry M00017. In an embodiment, the promoter polynucleotide sequences of SEQ ID NOs:1, 5 or 7 are functionally linked to one or more target genes of the valine/isoleucine biosynthesis pathway comprising genes of KEGG entry M00019. In an embodiment, the promoter polynucleotide sequences of SEQ ID NOs:1, 5 or 7 are functionally linked to one or more target genes of the isoleucine biosynthesis pathway comprising genes of KEGG entry M00535. In an embodiment, the promoter polynucleotide sequences of SEQ ID NOs:1, 5 or 7 are functionally linked to one or more target genes of the isoleucine biosynthesis pathway comprising genes of KEGG entry M00570. In an embodiment, the promoter polynucleotide sequences of SEQ ID NOs:1, 5 or 7 are functionally linked to one or more target genes of the leucine biosynthesis pathway comprising genes of KEGG entry M00432. In an embodiment, the promoter polynucleotide sequences of SEQ ID NOs:1, 5 or 7 are functionally linked to one or more target genes of the proline biosynthesis pathway comprising genes of KEGG entry M00015. In an embodiment, the promoter polynucleotide sequences of SEQ ID NOs:1, 5 or 7 are functionally linked to one or more target genes of the ornithine biosynthesis pathway comprising genes of KEGG entry M00028. In an embodiment, the promoter polynucleotide sequences of SEQ ID NOs:1, 5 or 7 are functionally linked to one or more target genes of the ornithine biosynthesis pathway comprising genes of KEGG entry M00763. In an embodiment, the promoter polynucleotide sequences of SEQ ID NOs:1, 5 or 7 are functionally linked to one or more target genes of the histidine biosynthesis pathway comprising genes of KEGG entry M00026. In an embodiment, the promoter polynucleotide sequences of SEQ ID NOs:1, 5 or 7 are functionally linked to one or more target genes of the shikimate biosynthesis pathway comprising genes of KEGG entry M00022. In an embodiment, the promoter polynucleotide sequences of SEQ ID NOs:1, 5 or 7 are functionally linked to one or more target genes of the tryptophan biosynthesis pathway comprising genes of entry M00023. In an embodiment, the promoter polynucleotide sequences of SEQ ID NOs:1, 5 or 7 are functionally linked to one or more target genes of the phenylalanine biosynthesis pathway comprising genes of KEGG entry M00024. In an embodiment, the promoter polynucleotide sequences of SEQ ID NOs:1, 5 or 7 are functionally linked to one or more target genes of the tyrosine biosynthesis pathway comprising genes of KEGG entry M00025. In an embodiment, the promoter polynucleotide sequences of SEQ ID NOs:1, 5 or 7 are functionally linked to one or more target genes of the tyrosine biosynthesis pathway comprising genes of KEGG entry M00040.

The present specification provides for, and includes, recombinant nucleic acid molecules comprising a promoter polynucleotide sequence selected from the group consisting of SEQ ID NOs:1 to 8 functionally linked to any one of the heterologous on- or off-pathway target genes from Corynebacterium glutamicum ATCC 13032 provided in Table 2 or any Corynebacterium glutamicum equivalent thereof. Sequence start and end positions correspond to genomic nucleotide accession NC 003450.3. It will be understood by those of ordinary skill in the art that corresponding genes exist in other strains of C. glutamicum and may be readily identified from Table 2. In an embodiment, the present specification provides for, and includes a recombinant nucleic acid molecule comprising a promoter polynucleotide sequence of SEQ ID NO:1 functionally linked to a heterologous target gene recited in Table 2. In an embodiment, the present specification provides for, and includes a recombinant nucleic acid molecule comprising a promoter polynucleotide sequence of SEQ ID NO:2 functionally linked to a heterologous target gene recited in Table 2. In an embodiment, the present specification provides for, and includes a recombinant nucleic acid molecule comprising a promoter polynucleotide sequence of SEQ ID NO:3 functionally linked to a heterologous target gene recited in Table 2. In an embodiment, the present specification provides for, and includes a recombinant nucleic acid molecule comprising a promoter polynucleotide sequence of SEQ ID NO:4 functionally linked to a heterologous target gene recited in Table 2. In an embodiment, the present specification provides for, and includes a recombinant nucleic acid molecule comprising a promoter polynucleotide sequence of SEQ ID NO:5 functionally linked to a heterologous target gene recited in Table 2. In an embodiment, the present specification provides for, and includes a recombinant nucleic acid molecule comprising a promoter polynucleotide sequence of SEQ ID NO:6 functionally linked to a heterologous target gene recited in Table 2. In an embodiment, the present specification provides for, and includes a recombinant nucleic acid molecule comprising a promoter polynucleotide sequence of SEQ ID NO:7 functionally linked to a heterologous target gene recited in Table 2. In an embodiment, the present specification provides for, and includes a recombinant nucleic acid molecule comprising a promoter polynucleotide sequence of SEQ ID NO:8 functionally linked to a heterologous target gene recited in Table 2.

TABLE 2
Target genes from Corynebacterium glutamicum according to the present specification

| Gene ID | Symbol | Aliases | Description | start | end | orientation |
|---|---|---|---|---|---|---|
| 1021315 | NCgl0248 | NCgl0248, | aspartate-semialdehyde | 270660 | 271694 | plus |
| | | Cgl0252 | dehydrogenase | | | |
| 1021300 | NCgl0223 | NCgl0223, | prephenate dehydrogenase | 241880 | 242902 | minus |
| | | Cgl0226 | | | | |
| 1021294 | NCgl0247 | NCgl0247, | aspartate kinase | 269371 | 270636 | plus |
| | | Cgl0251 | | | | |
| 1021282 | NCgl0215 | NCgl0215, | Aminotransferase | 232257 | 233282 | minus |
| | | Cgl0218 | | | | |
| 1021250 | NCgl0181 | NCgl0181, | glutamine 2-oxoglutarate | 195240 | 199772 | plus |
| | | Cgl0184 | aminotransferase large | | | |
| | | | subunit | | | |
| 1021247 | gltD | NCgl0182, | glutamate synthase | 199772 | 201292 | plus |
| | | Cgl0185 | | | | |
| 1021203 | aroE | NCgl0409, | quinate/shikimate | 446538 | 447389 | plus |
| | | Cgl0424 | dehydrogenase | | | |
| 1021149 | NCgl0245 | NCgl0245, | 2-isopropylmalate synthase | 266151 | 267896 | minus |
| | | Cgl0248 | | | | |
| 1021136 | gpmA | NCgl0390, | 2,3-bisphosphoglycerate- | 425177 | 425923 | plus |
| | | Cgl0402 | dependent phosphoglycerate | | | |
| | | | mutase | | | |
| 1021131 | NCgl0408 | NCgl0408, | 3-dehydroquinate | 446087 | 446524 | plus |
| | | Cgl0423 | dehydratase | | | |
| 1021078 | NCgl0398 | NCgl0398, | pyrroline-5-carboxylate | 434877 | 435698 | plus |
| | | Cgl0410 | reductase | | | |
| 1020978 | trpA | NCgl2932, | tryptophan synthase subunit | 3239333 | 3240175 | plus |
| | | Cgl3035 | alpha | | | |
| 1020976 | NCgl2931 | NCgl2931, | tryptophan synthase subunit | 3238083 | 3239336 | plus |
| | | Cgl3034 | beta | | | |
| 1020975 | NCgl2930 | NCgl2930, | bifunctional indole-3- | 3236642 | 3238066 | plus |
| | | trpC, trpF | glycerol phosphate | | | |
| | | | synthase/phospho- | | | |
| | | | ribosylanthranilate | | | |
| | | | isomerase | | | |
| 1020974 | trpD | NCgl2929, | anthranilate | 3235603 | 3236649 | plus |
| | | Cgl3032 | phosphoribosyltransferase | | | |
| 1020973 | NCgl2928 | NCgl2928, | anthranilate synthase II | 3234957 | 3235583 | plus |
| | | Cgl3031 | | | | |
| 1020972 | NCgl2927 | NCgl2927, | anthranilate synthase I | 3233404 | 3234960 | plus |
| | | Cgl3029 | | | | |
| 1020852 | NCgl2809 | NCgl2809, | pyruvate kinase | 3110462 | 3112321 | minus |
| | | Cgl2910 | | | | |
| 1020842 | NCgl2799 | NCgl2799, | prephenate dehydratase | 3098576 | 3099523 | minus |
| | | Cgl2899 | | | | |
| 1020841 | NCgl2798 | NCgl2798, | phosphoglycerate mutase | 3097902 | 3098573 | minus |
| | | Cgl2898 | | | | |
| 1020788 | NCgl2747 | NCgl2747, | Aminotransferase | 3030670 | 3031983 | plus |
| | | Cgl2844 | | | | |
| 1020745 | NCgl2704 | NCgl2704, | Nucleosidase | 2988212 | 2988772 | minus |
| | | Cgl2802 | | | | |
| 1020729 | NCgl2688 | NCgl2688, | cystathionine gamma- | 2972058 | 2973206 | minus |
| | | Cgl2786 | synthase | | | |
| 1020714 | NCgl2673 | NCgl2673, | fructose-bisphosphate | 2954239 | 2955273 | minus |
| | | Cgl2770 | aldolase | | | |
| 1020594 | NCgl2557 | NCgl2557, | dihydrodipicolinate | 2815459 | 2816397 | plus |
| | | Cgl2646 | synthase | | | |
| 1020564 | NCgl2528 | NCgl2528, | D-2-hydroxyisocaproate | 2786754 | 2787716 | minus |
| | | Cgl2617 | dehydrogenase | | | |
| 1020509 | NCgl2474 | NCgl2474, | serine acetyltransferase | 2723065 | 2723613 | plus |
| | | Cgl2563 | | | | |
| 1020508 | NCgl2473 | NCgl2473, | cysteine synthase | 2721905 | 2722861 | plus |
| | | Cgl2562 | | | | |
| 1020471 | NCgl2436 | NCgl2436, | phosphoserine phosphatase | 2669555 | 2670856 | minus |
| | | Cgl2522 | | | | |
| 1020393 | NCgl2360 | NCgl2360, | cystathionine gamma- | 2590310 | 2591470 | minus |
| | | Cgl2446 | synthase | | | |
| 1020370 | NCgl2337 | NCgl2337, | ribose-5-phosphate | 2563930 | 2564403 | minus |
| | | Cgl2423 | isomerase B | | | |
| 1020307 | NCgl2274 | NCgl2274, | gamma-glutamyl kinase | 2496668 | 2497777 | minus |
| | | Cgl2356 | | | | |
| 1020305 | proA | NCgl2272, | gamma-glutamyl phosphate | 2494337 | 2495635 | minus |
| | | Cgl2354 | reductase | | | |
| 1020301 | NCgl2268 | NCgl2268, | fructose-2,6-bisphosphatase | 2491149 | 2491859 | minus |
| | | Cgl2350 | | | | |
| 1020260 | NCgl2227 | NCgl2227, | PLP-dependent | 2444607 | 2445713 | plus |
| | | Cgl2309 | aminotransferase | | | |
| 1020188 | NCgl2155 | NCgl2155, | bifunctional RNase H/acid | 2371410 | 2372558 | minus |
| | | Cgl2236 | phosphatase | | | |
| 1020181 | NCgl2148 | NCgl2148, | glutamine synthase | 2362816 | 2364156 | minus |
| | | Cgl2229 | | | | |
| 1020172 | NCgl2139 | NCgl2139, | threonine synthase | 2353598 | 2355043 | minus |
| | | Cgl2220 | | | | |
| 1020166 | NCgl2133 | NCgl2133, | glutamine synthase | 2348830 | 2350263 | plus |
| | | Cgl2214 | | | | |
| 1020155 | NCgl2123 | NCgl2123, | branched-chain amino acid | 2335913 | 2337016 | minus |
| | | Cgl2204 | aminotransferase | | | |
| 1020130 | NCgl2098 | NCgl2098, | 3-deoxy-7- | 2307695 | 2309095 | minus |
| | | Cgl2178 | phosphoheptulonate | | | |
| | | | synthase | | | |
| 1020087 | NCgl2055 | NCgl2055, | cysteine synthase | 2258360 | 2259313 | minus |
| | | Cgl2136 | | | | |
| 1020086 | NCgl2054 | NCgl2054, | diaminopimelate | 2255736 | 2257025 | minus |
| | | Cgl2135 | decarboxylase | | | |
| 1020080 | NCgl2048 | NCgl2048, | methionine synthase II | 2247004 | 2248209 | minus |
| | | Cgl2129 | | | | |
| 1020078 | NCgl2046 | NCgl2046, | threonine dehydratase | 2244862 | 2246172 | minus |
| | | Cgl2127 | | | | |
| 1020053 | hisD | NCgl2021, | histidinol dehydrogenase | 2217597 | 2218925 | minus |
| | | Cgl2102 | | | | |
| 1020052 | NCgl2020 | NCgl2020, | histidinol-phosphate | 2216491 | 2217591 | minus |
| | | Cgl2101 | aminotransferase | | | |
| 1020051 | hisB | NCgl2019, | imidazoleglycerol- | 2215866 | 2216474 | minus |
| | | Cgl2100 | phosphate dehydratase | | | |
| 1020048 | hisH | NCgl2016, | imidazole glycerol | 2212638 | 2213273 | minus |
| | | Cgl2097 | phosphate synthase subunit | | | |
| | | | HisH | | | |
| 1020047 | NCgl2015 | NCgl2015, | phosphoribosyl isomerase A | 2211879 | 2212619 | minus |
| | | Cgl2096 | | | | |
| 1020045 | hisF | NCgl2013, | imidazole glycerol | 2210270 | 2211046 | minus |
| | | Cgl2094 | phosphate synthase subunit | | | |
| | | | HisF | | | |
| 1020044 | hisI | NCgl2012, | phosphoribosyl-AMP | 2209917 | 2210273 | minus |
| | | Cgl2093 | cyclohydrolase | | | |
| 1020042 | NCgl2010 | NCgl2010, | indole-3-glycerol phosphate | 2208364 | 2209149 | minus |
| | | Cgl2091 | synthase | | | |
| 1020040 | NCgl2008 | NCgl2008, | pyruvate kinase | 2205665 | 2207092 | minus |
| | | Cgl2089 | | | | |
| 1019930 | NCgl1898 | NCgl1898, | 4-hydroxy- | 2081188 | 2081934 | minus |
| | | Cgl1973 | tetrahydrodipicolinate | | | |
| | | | reductase | | | |
| 1019928 | dapA | NCgl1896, | 4-hydroxy- | 2079278 | 2080183 | minus |
| | | Cgl1971 | tetrahydrodipicolinate | | | |
| | | | synthase | | | |
| 1019900 | dapF | NCgl1868, | diaminopimelate epimerase | 2051842 | 2052675 | minus |
| | | Cgl1943 | | | | |
| 1019614 | NCgl1583 | NCgl1583, | L-serine deaminase | 1744884 | 1746233 | plus |
| | | Cgl1645 | | | | |
| 1019598 | aroE | NCgl1567, | shikimate 5-dehydrogenase | 1724609 | 1725439 | minus |
| | | Cgl1629 | | | | |
| 1019592 | NCgl1561 | NCgl1561, | chorismate synthase | 1719666 | 1720898 | minus |
| | | Cgl1623 | | | | |
| 1019591 | aroK | NCgl1560, | shikimate kinase | 1719104 | 1719676 | minus |
| | | Cgl1622 | | | | |
| 1019590 | aroB | NCgl1559, | 3-dehydroquinate synthase | 1717935 | 1719032 | minus |
| | | Cgl1621 | | | | |
| 1019571 | NCgl1541 | NCgl1541, | methionine | 1699174 | 1700397 | minus |
| | | Cgl1603 | adenosyltransferase | | | |
| 1019566 | NCgl1536 | NCgl1536, | ribulose-phosphate 3- | 1693259 | 1693918 | minus |
| | | Cgl1598 | epimerase | | | |
| 1019556 | NCgl1526 | NCgl1526, | glyceraldehyde-3-phosphate | 1682621 | 1683625 | minus |
| | | Cgl1588 | dehydrogenase | | | |
| 1019555 | pgk | NCgl1525, | phosphoglycerate kinase | 1681187 | 1682404 | minus |
| | | Cgl1587 | | | | |
| 1019554 | tpiA | NCgl1524, | triosephosphate isomerase | 1680329 | 1681108 | minus |
| | | Cgl1586 | | | | |
| 1019550 | NCgl1520 | NCgl1520, | ornithine cyclodeaminase | 1674120 | 1675268 | minus |
| | | Cgl1582 | | | | |
| 1019543 | NCgl1513 | NCgl1513, | Transaldolase | 1666673 | 1667755 | plus |
| | | Cgl1575 | | | | |
| 1019542 | NCgl1512 | NCgl1512, | Transketolase | 1664403 | 1666505 | plus |
| | | Cgl1574 | | | | |
| 1019512 | NCgl1482 | NCgl1482, | aconitate hydratase | 1626279 | 1629110 | plus |
| | | Cgl1540 | | | | |
| 1019480 | NCgl1450 | NCgl1450, | methionine synthase I | 1587570 | 1591235 | minus |
| | | Cgl1507 | cobalamin-binding subunit | | | |
| 1019478 | hisE | NCgl1448, | phosphoribosyl-ATP | 1586462 | 1586725 | minus |
| | | Cgl1505 | pyrophosphatase | | | |
| 1019477 | hisG | NCgl1447, | ATP | 1585600 | 1586445 | minus |
| | | Cgl1504 | phosphoribosyltransferase | | | |
| 1019377 | NCgl1347 | NCgl1347, | argininosuccinate lyase | 1471477 | 1472910 | plus |
| | | Cgl1401 | | | | |
| 1019376 | NCgl1346 | NCgl1346, | argininosuccinate synthase | 1470211 | 1471416 | plus |
| | | Cgl1400 | | | | |
| 1019374 | NCgl1344 | NCgl1344, | ornithine | 1468565 | 1469524 | plus |
| | | Cgl1398 | carbamoyltransferase | | | |
| 1019373 | argD | NCgl1343, | acetylornithine | 1467376 | 1468551 | plus |
| | | Cgl1397 | aminotransferase | | | |
| 1019372 | NCgl1342 | NCgl1342, | acetylglutamate kinase | 1466422 | 1467375 | plus |
| | | Cgl1396 | | | | |
| 1019371 | argJ | NCgl1341, | bifunctional ornithine | 1465210 | 1466376 | plus |
| | | Cgl1395 | acetyltransferase/N- | | | |
| | | | acetylglutamate synthase | | | |
| 1019370 | argC | NCgl1340, | N-acetyl-gamma-glutamyl- | 1464053 | 1465126 | plus |
| | | Cgl1394 | phosphate reductase | | | |
| 1019293 | leuD | NCgl1263, | 3-isopropylmalate | 1381902 | 1382495 | plus |
| | | Cgl1316 | dehydratase small subunit | | | |
| 1019292 | NCgl1262 | NCgl1262, | 3-isopropylmalate | 1380440 | 1381885 | plus |
| | | Cgl1315 | dehydratase large subunit | | | |
| 1019267 | NCgl1237 | NCgl1237, | 3-isopropylmalate | 1353489 | 1354511 | plus |
| | | Cgl1286 | dehydrogenase | | | |
| 1019265 | NCgl1235 | NCgl1235, | D-3-phosphoglycerate | 1350855 | 1352447 | plus |
| | | Cgl1284 | dehydrogenase | | | |
| 1019254 | NCgl1224 | NCgl1224, | ketol-acid reductoisomerase | 1340724 | 1341740 | plus |
| | | Cgl1273 | | | | |
| 1019253 | ilvH | NCgl1223, | acetolactate synthase small | 1340025 | 1340543 | plus |
| | | Cgl1272 | subunit | | | |
| 1019252 | NCgl1222 | NCgl1222, | acetolactate synthase large | 1338131 | 1340011 | plus |
| | | Cgl1271 | subunit | | | |
| 1019249 | NCgl1219 | NCgl1219, | dihydroxy-acid dehydratase | 1333439 | 1335280 | minus |
| | | Cgl1268 | | | | |
| 1019232 | NCgl1202 | NCgl1202, | 6-phosphofructokinase | 1315046 | 1316086 | plus |
| | | Cgl1250 | | | | |
| 1019167 | NCgl1137 | NCgl1137, | homoserine kinase | 1243855 | 1244784 | plus |
| | | Cgl1184 | | | | |
| 1019166 | NCgl1136 | NCgl1136, | homoserine dehydrogenase | 1242507 | 1243844 | plus |
| | | Cgl1183 | | | | |
| 1019163 | NCgl1133 | NCgl1133, | diaminopimelate | 1239929 | 1241266 | plus |
| | | Cgl1180 | decarboxylase | | | |
| 1019124 | NCgl1094 | NCgl1094, | 5- | 1188385 | 1190622 | minus |
| | | Cgl1139 | methyltetrahydropteroyl- | | | |
| | | | triglutamate--homocysteine S- | | | |
| | | | methyltransferase | | | |
| 1019117 | aroE | NCgl1087, | shikimate 5-dehydrogenase | 1180869 | 1181675 | minus |
| | | Cgl1132 | | | | |
| 1019094 | NCgl1064 | NCgl1064, | succinyl-diaminopimelate | 1155731 | 1156840 | plus |
| | | Cgl1109 | desuccinylase | | | |
| 1019093 | NCgl1063 | NCgl1063, | tetrahydrodipicolinate N- | 1154726 | 1155676 | minus |
| | | Cgl1108 | succinyltransferase | | | |
| 1019091 | NCgl1061 | NCgl1061, | 2,3,4,5-tetrahydropyridine- | 1152370 | 1153263 | minus |
| | | Cgl1106 | 2,6-dicarboxylate N- | | | |
| | | | succinyltransferase | | | |
| 1019042 | NCgl1013 | NCgl1013, | phosphoglycerate mutase | 1107503 | 1108204 | plus |
| | | Cgl1058 | | | | |
| 1018983 | glyA | NCgl0954, | serine | 1050624 | 1051928 | plus |
| | | Cgl0996 | hydroxymethyltransferase | | | |
| 1018979 | NCgl0950 | NCgl0950, | phospho-2-dehydro-3- | 1046610 | 1047710 | plus |
| | | Cgl0990 | deoxyheptonate aldolase | | | |
| 1018968 | NCgl0939 | NCgl0939, | threonine dehydratase | 1038718 | 1039650 | minus |
| | | Cgl0978 | | | | |
| 1018964 | eno | NCgl0935, | phosphopyruvate hydratase | 1034949 | 1036226 | plus |
| | | Cgl0974 | | | | |
| 1018934 | NCgl0905 | NCgl0905, | ribose-phosphate | 997463 | 998440 | minus |
| | | Cgl0942 | pyrophosphokinase | | | |
| 1018929 | NCgl0900 | NCgl0900, | glyceraldehyde-3-phosphate | 993174 | 994616 | plus |
| | | Cgl0937 | dehydrogenase | | | |
| 1018848 | NCgl0819 | NCgl0819, | hypothetical protein | 910852 | 911157 | minus |
| | | Cgl0853 | | | | |
| 1018824 | gltA | NCgl0795, | type II citrate synthase | 877838 | 879151 | plus |
| | | Cgl0829 | | | | |
| 1018823 | NCgl0794 | NCgl0794, | phosphoserine | 875982 | 877112 | minus |
| | | Cgl0828 | aminotransferase | | | |
| 1018809 | NCgl0780 | NCgl0780, | Aminotransferase | 861592 | 862755 | plus |
| | | Cgl0814 | | | | |
| 1018794 | NCgl0765 | NCgl0765, | fructose-1,6-bisphosphatase | 841514 | 842296 | minus |
| | | Cgl0799 | | | | |
| 1018759 | NCgl0730 | NCgl0730, | 3-phosphoshikimate 1- | 801187 | 802479 | minus |
| | | Cgl0764 | carboxyvinyltransferase | | | |
| 1018688 | NCgl0659 | NCgl0659, | pyruvate carboxylase | 705211 | 708633 | plus |
| | | Cgl0689 | | | | |
| 1018663 | NCgl0634 | NCgl0634, | monomeric isocitrate | 677828 | 680044 | minus |
| | | Cgl0664 | dehydrogenase (NADP+) | | | |

In an embodiment, the present specification provides for, and includes a recombinant vector comprising a promoter polynucleotide sequence of SEQ ID NO:1 functionally linked to an on- or off-pathway heterologous target gene recited in Table 2. In an embodiment, the present specification provides for, and includes a recombinant vector comprising a promoter polynucleotide sequence of SEQ ID NO:2 functionally linked to a heterologous target gene recited in Table 2. In an embodiment, the present specification provides for, and includes a recombinant vector comprising a promoter polynucleotide sequence of SEQ ID NO:3 functionally linked to a heterologous target gene recited in Table 2. In an embodiment, the present specification provides for, and includes a recombinant vector comprising a promoter polynucleotide sequence of SEQ ID NO:4 functionally linked to a heterologous target gene recited in Table 2. In an embodiment, the present specification provides for, and includes a recombinant vector comprising a promoter polynucleotide sequence of SEQ ID NO:5 functionally linked to a heterologous target gene recited in Table 2. In an embodiment, the present specification provides for, and includes a recombinant vector comprising a promoter polynucleotide sequence of SEQ ID NO:6 functionally linked to a heterologous target gene recited in Table 2. In an embodiment, the present specification provides for, and includes a recombinant vector comprising a promoter polynucleotide sequence of SEQ ID NO:7 functionally linked to a heterologous target gene recited in Table 2. In an embodiment, the present specification provides for, and includes a recombinant vector comprising a promoter polynucleotide sequence of SEQ ID NO:8 functionally linked to a heterologous target gene recited in Table 2.

In an embodiment, the present specification provides for, and includes, a host cell transformed with a recombinant vector comprising a promoter polynucleotide sequence of SEQ ID NO:1 functionally linked to an on- or off-pathway heterologous target gene recited in Table 2. In an embodiment, the present specification provides for, and includes, a host cell transformed with a recombinant vector comprising a promoter polynucleotide sequence of SEQ ID NO:2 functionally linked to a heterologous target gene recited in Table 2. In an embodiment, the present specification provides for, and includes, a host cell transformed with a recombinant vector comprising a promoter polynucleotide sequence of SEQ ID NO:3 functionally linked to a heterologous target gene recited in Table 2. In an embodiment, the present specification provides for, and includes, a host cell transformed with a recombinant vector comprising a promoter polynucleotide sequence of SEQ ID NO:4 functionally linked to a heterologous target gene recited in Table 2. In an embodiment, the present specification provides for, and includes, a host cell transformed with a recombinant vector comprising a promoter polynucleotide sequence of SEQ ID NO:5 functionally linked to a heterologous target gene recited in Table 2. In an embodiment, the present specification provides for, and includes, a host cell transformed with a recombinant vector comprising a promoter polynucleotide sequence of SEQ ID NO:6 functionally linked to a heterologous target gene recited in Table 2. In an embodiment, the present specification provides for, and includes, a host cell transformed with a recombinant vector comprising a promoter polynucleotide sequence of SEQ ID NO:7 functionally linked to a heterologous target gene recited in Table 2. In an embodiment, the present specification provides for, and includes, a host cell transformed with a recombinant vector comprising a promoter polynucleotide sequence of SEQ ID NO:8 functionally linked to a heterologous target gene recited in Table 2.

In an embodiment, the present specification provides for, and includes, a host cell transformed with a recombinant vector comprising a promoter polynucleotide sequence of SEQ ID NO:1 functionally linked to an on- or off-pathway heterologous target gene recited in Table 3. In an embodiment, the present specification provides for, and includes, a host cell transformed with a recombinant vector comprising a promoter polynucleotide sequence of SEQ ID NO:2 functionally linked to a heterologous target gene recited in Table 3. In an embodiment, the present specification provides for, and includes, a host cell transformed with a recombinant vector comprising a promoter polynucleotide sequence of SEQ ID NO:3 functionally linked to a heterologous target gene recited in Table 3. In an embodiment, the present specification provides for, and includes, a host cell transformed with a recombinant vector comprising a promoter polynucleotide sequence of SEQ ID NO:4 functionally linked to a heterologous target gene recited in Table 3. In an embodiment, the present specification provides for, and includes, a host cell transformed with a recombinant vector comprising a promoter polynucleotide sequence of SEQ ID NO:5 functionally linked to a heterologous target gene recited in Table 3. In an embodiment, the present specification provides for, and includes, a host cell transformed with a recombinant vector comprising a promoter polynucleotide sequence of SEQ ID NO:6 functionally linked to a heterologous target gene recited in Table 3. In an embodiment, the present specification provides for, and includes, a host cell transformed with a recombinant vector comprising a promoter polynucleotide sequence of SEQ ID NO:7 functionally linked to a heterologous target gene recited in Table 3. In an embodiment, the present specification provides for, and includes, a host cell transformed with a recombinant vector comprising a promoter polynucleotide sequence of SEQ ID NO:8 functionally linked to a heterologous target gene recited in Table 3.

TABLE 3
C. glutamican L-lysine Biosynthetic Pathway

| | | C. Glutamicum | | |
|---|---|---|---|---|
| Symbol | Gene Name (EC #) | Gene | Position | Expression |
| Asd | aspartate-semialdehyde | Asd | 270660 . . . 271694 | + |
| | dehydrogenase (EC: 1.2.1.11) | | | |
| dapA | 4-hydroxy-tetrahydrodipicolinate | dapA | Complement | + |
| | synthase (EC: 4.3.3.7) | | (2079278 . . . 2080183) | |
| dapB | dihydrodipicolinate reductase | Cgl1973 | complement | + |
| | (EC: 1.17.1.8) | | (2081188 . . . 2081934) | |
| dapD | 2,3,4,5-tetrahydropyridine-2- | dapD | complement | + |
| | carboxylate N- | | (1153838 . . . 1154731) | |
| | succinyltransferase | | | |
| | (EC: 2.3.1.117) | | | |
| dapD | 2,3,4,5-tetrahydropyridine-2- | dapD2 | complement | |
| | carboxylate N- | | (1156194 . . . 1157144) | |
| | succinyltransferase | | | |
| | (EC: 2.3.1.117) | | | |
| cg0931 | N-succinyldiaminopimelate | cg0931 | 863063 . . . 864226 | + |
| | aminotransferase (EC: 2.6.1.17) | | | |
| dapE | succinyl-diaminopimelate | dapE | 1157199 . . . 1158308 | + |
| | desuccinylase (EC: 3.5.1.18) | | | |
| dapF | diaminopimelate epimerase | dapF | complement | + |
| | (EC: 5.1.1.7) | | (2021891 . . . 2022724) | |
| lysA | diaminopimelate decarboxylase | lysA | 1241397 . . . 1242734 | + |
| | (EC: 4.1.1.20) | | | |
| ddh | diaminopimelate dehydrogenase | Ddh | complement | + |
| | (EC: 1.4.1.16) | | (2760062 . . . 2761024) | |
| ask (lysC) | Aspartokinase Lysc Alpha And | lysC | 269371 . . . 270636 | + |
| | Beta Subunits (EC: 2.7.2.4) | | | |
| aspB | Aspartate Aminotransferase | aspB | 256618 . . . 257898 | + |
| | (EC: 2.6.1.1) | | | |
| PTS | Phosphotransferase System | ptsG | 1424684 . . . 1426735 | + |
| | (PTS); Glucose-Specific Enzyme | | | |
| | II BC Component Of PTS | | | |
| | (EC: 2.7.1.69) | | | |
| zwf | glucose-6-phosphate 1- | Zwf | 1669327 . . . 1670871 | + |
| | dehydrogenase (EC: 1.1.1.49 | | | |
| | 1.1.1.363) | | | |
| pgi | glucose-6-phosphate isomerase | Pgi | complement | + |
| | (EC: 5.3.1.9) | | (909227 . . . 910849) | |
| Tkt | transketolase (EC: 2.2.1.1) | Tkt | 1665870 . . . 1667972 | + |
| fbp | 6-phosphofructokinase 1 | Cgl1250 | 1315046 . . . 1316086 | + |
| | (EC: 2.7.1.11) | | | |
| ppc | phosphoenolpyruvate | ppc | complement | + |
| | carboxylase (EC: 4.1.1.31) | | (1678851 . . . 1681610) | |
| pyc | pyruvate carboxylase | pyc | 706684 . . . 710106 | + |
| | (EC: 6.4.1.1) | | | |
| icd | isocitrate dehydrogenase | Icd | complement | − |
| | (EC: 1.1.1.42) | | (679301 . . . 681517) | |
| pck | phosphoenolpyruvate | pck | complement | − |
| | carboxykinase (GTP) | | (3025365 . . . 3027197) | |
| | (EC: 4.1.1.32) | | | |
| odx | Oxaloacetate decarboxylase | odx | AP017369.1: | − |
| | (EC 4.1.1.3) | | 1508967 . . . 1509782 | |
| | | | (from C. glutamicum | |
| | | | N24) | |
| hom | homoserine kinase (EC: 2.7.1.39) | Cgl1184 | 1243855 . . . 1244784 | − |
| | homoserine dehydrogenase | Cgl1183 | 1242507 . . . 1243844 | − |
| | (EC: 1.1.1.3); | | | |
| | threonine synthase | Cgl2220 | complement | − |
| | (EC: 4.2.3.1) | | (2353598 . . . 2355043) | |

In an embodiment, the present specification provides for, and includes a recombinant vector comprising a promoter polynucleotide sequence of SEQ ID NO:1 functionally linked to an on- or off-pathway heterologous target gene recited in Table 3. In an embodiment, the present specification provides for, and includes a recombinant vector comprising a promoter polynucleotide sequence of SEQ ID NO:2 functionally linked to a heterologous target gene recited in Table 3. In an embodiment, the present specification provides for, and includes a recombinant vector comprising a promoter polynucleotide sequence of SEQ ID NO:3 functionally linked to a heterologous target gene recited in Table 3. In an embodiment, the present specification provides for, and includes a recombinant vector comprising a promoter polynucleotide sequence of SEQ ID NO:4 functionally linked to a heterologous target gene recited in Table 3. In an embodiment, the present specification provides for, and includes a recombinant vector comprising a promoter polynucleotide sequence of SEQ ID NO:5 functionally linked to a heterologous target gene recited in Table 3. In an embodiment, the present specification provides for, and includes a recombinant vector comprising a promoter polynucleotide sequence of SEQ ID NO:6 functionally linked to a heterologous target gene recited in Table 3. In an embodiment, the present specification provides for, and includes a recombinant vector comprising a promoter polynucleotide sequence of SEQ ID NO:7 functionally linked to a heterologous target gene recited in Table 3. In an embodiment, the present specification provides for, and includes a recombinant vector comprising a promoter polynucleotide sequence of SEQ ID NO:8 functionally linked to a heterologous target gene recited in Table 3.

In an embodiment, the present specification provides for, and includes, a host cell transformed with a recombinant vector comprising a promoter polynucleotide sequence of SEQ ID NO:1 functionally linked to an on or off-pathway heterologous target gene recited in Table 3. In an embodiment, the present specification provides for, and includes, a host cell transformed with a recombinant vector comprising a promoter polynucleotide sequence of SEQ ID NO:2 functionally linked to a heterologous target gene recited in Table 3. In an embodiment, the present specification provides for, and includes, a host cell transformed with a recombinant vector comprising a promoter polynucleotide sequence of SEQ ID NO:3 functionally linked to a heterologous target gene recited in Table 3. In an embodiment, the present specification provides for, and includes, a host cell transformed with a recombinant vector comprising a promoter polynucleotide sequence of SEQ ID NO:4 functionally linked to a heterologous target gene recited in Table 3. In an embodiment, the present specification provides for, and includes, a host cell transformed with a recombinant vector comprising a promoter polynucleotide sequence of SEQ ID NO:5 functionally linked to a heterologous target gene recited in Table 3. In an embodiment, the present specification provides for, and includes, a host cell transformed with a recombinant vector comprising a promoter polynucleotide sequence of SEQ ID NO:6 functionally linked to a heterologous target gene recited in Table 3. In an embodiment, the present specification provides for, and includes, a host cell transformed with a recombinant vector comprising a promoter polynucleotide sequence of SEQ ID NO:7 functionally linked to a heterologous target gene recited in Table 3. In an embodiment, the present specification provides for, and includes, a host cell transformed with a recombinant vector comprising a promoter polynucleotide sequence of SEQ ID NO:8 functionally linked to a heterologous target gene recited in Table 3.

The present specification provides for a host cell transformed with a recombinant vector comprising a promoter polynucleotide sequence selected from the group consisting of SEQ ID NOs:1 to 8 functionally linked to any one of the on- or off-pathway heterologous target genes from Corynebacterium glutamicum ATCC 13032 provided in Table 4 or their Corynebacterium glutamicum equivalent thereof. Sequence start and end positions correspond to genomic nucleotide accession NC 003450.3. It will be understood by those of ordinary skill in the art that corresponding genes exist in other strains of C. glutamicum and may be readily identified from Table 4. In an embodiment, the present specification provides for, and includes, a host cell transformed with a recombinant vector comprising a promoter polynucleotide sequence of SEQ ID NO:1 functionally linked to a heterologous target gene recited in Table 4. In an embodiment, the present specification provides for, and includes, a host cell transformed with a recombinant vector comprising a promoter polynucleotide sequence of SEQ ID NO:2 functionally linked to a heterologous target gene recited in Table 4. In an embodiment, the present specification provides for, and includes, a host cell transformed with a recombinant vector comprising a promoter polynucleotide sequence of SEQ ID NO:3 functionally linked to a heterologous target gene recited in Table 4. In an embodiment, the present specification provides for, and includes, a host cell transformed with a recombinant vector comprising a promoter polynucleotide sequence of SEQ ID NO:4 functionally linked to a heterologous target gene recited in Table 4. In an embodiment, the present specification provides for, and includes, a host cell transformed with a recombinant vector comprising a promoter polynucleotide sequence of SEQ ID NO:5 functionally linked to a heterologous target gene recited in Table 4. In an embodiment, the present specification provides for, and includes, a host cell transformed with a recombinant vector comprising a promoter polynucleotide sequence of SEQ ID NO:6 functionally linked to a heterologous target gene recited in Table 4. In an embodiment, the present specification provides for, and includes, a host cell transformed with a recombinant vector comprising a promoter polynucleotide sequence of SEQ ID NO:7 functionally linked to a heterologous target gene recited in Table 4. In an embodiment, the present specification provides for, and includes, a host cell transformed with a recombinant vector comprising a promoter polynucleotide sequence of SEQ ID NO:8 functionally linked to a heterologous target gene recited in Table 4.

TABLE 4
C. glutamican L-methionine Biosynthetic Pathway

| | | C. Glutamicum | |
|---|---|---|---|
| Symbol | Gene Name (EC #) | Gene | Position |
| lysC | aspartate kinase | Cgl0251 | 269371 . . . 270636 |
| | [EC: 2.7.2.4] | | |
| | aspartate- | Cgl0252 | 270660 . . . 271694 |
| | semialdehyde | | |
| | dehydrogenase | | |
| | [EC: 1.2.1.11] | | |
| dapA | 4-hydroxy- | dapA | complement |
| | tetrahydro- | | (2079278 . . . 2080183) |
| | dipicolinate | | |
| | synthase | | |
| | [EC: 4.3.3.7] | | |
| dapA | 4-hydroxy- | Cgl2646 | 2815459 . . . 2816397 |
| | tetrahydro- | | |
| | dipicolinate | | |
| | synthase | | |
| | [EC: 4.3.3.7] | | |
| dapB | 4-hydroxy- | Cgl1973 | complement |
| | tetrahydro- | | (2081188 . . . 2081934) |
| | dipicolinate | | |
| | reductase | | |
| | [EC: 1.17.1.8] | | |
| dapD | 2,3,4,5- | Cgl1106 | complement |
| | tetrahydro- | | (1152370 . . . 1153263) |
| | pyridine-2- | | |
| | carboxylate N- | | |
| | succinyltransferase | | |
| | [EC: 2.3.1.117] | | |
| dapC | N- | Cgl0814 | 861592 . . . 862755 |
| | succinyldiamino- | | |
| | pimelate | | |
| | aminotransferase | | |
| | [EC: 2.6.1.17] | | |
| dapE | succinyl- | Cgl1109 | 1155731 . . . 1156840 |
| | diaminopimelate | | |
| | desuccinylase | | |
| | [EC: 3.5.1.18] | | |
| dapF | diaminopimelate | dapF | complement |
| | epimerase | | (2051842 . . . 2052675) |
| | [EC: 5.1.1.7] | | |
| lysA | diaminopimelate | Cgl1180 | 1239929 . . . 1241266 |
| | decarboxylase | | |
| | [EC: 4.1.1.20] | | |

In an embodiment, the present specification provides for, and includes a recombinant vector comprising a promoter polynucleotide sequence of SEQ ID NO:3 functionally linked to an on- or off-pathway heterologous target gene recited in Table 4. In an embodiment, the present specification provides for, and includes a recombinant vector comprising a promoter polynucleotide sequence of SEQ ID NO:4 functionally linked to a heterologous target gene recited in Table 4. In an embodiment, the present specification provides for, and includes a recombinant vector comprising a promoter polynucleotide sequence of SEQ ID NO:5 functionally linked to a heterologous target gene recited in Table 4. In an embodiment, the present specification provides for, and includes a recombinant vector comprising a promoter polynucleotide sequence of SEQ ID NO:6 functionally linked to a heterologous target gene recited in Table 4. In an embodiment, the present specification provides for, and includes a recombinant vector comprising a promoter polynucleotide sequence of SEQ ID NO:7 functionally linked to a heterologous target gene recited in Table 4. In an embodiment, the present specification provides for, and includes a recombinant vector comprising a promoter polynucleotide sequence of SEQ ID NO:8 functionally linked to a heterologous target gene recited in Table 4.

In an embodiment, the present specification provides for, and includes, a host cell transformed with a recombinant vector comprising a promoter polynucleotide sequence of SEQ ID NO:1 functionally linked to an on- or off-pathway heterologous target gene recited in Table 4. In an embodiment, the present specification provides for, and includes, a host cell transformed with a recombinant vector comprising a promoter polynucleotide sequence of SEQ ID NO:2 functionally linked to a heterologous target gene recited in Table 4. In an embodiment, the present specification provides for, and includes, a host cell transformed with a recombinant vector comprising a promoter polynucleotide sequence of SEQ ID NO:3 functionally linked to a heterologous target gene recited in Table 4. In an embodiment, the present specification provides for, and includes, a host cell transformed with a recombinant vector comprising a promoter polynucleotide sequence of SEQ ID NO:4 functionally linked to a heterologous target gene recited in Table 4. In an embodiment, the present specification provides for, and includes, a host cell transformed with a recombinant vector comprising a promoter polynucleotide sequence of SEQ ID NO:5 functionally linked to a heterologous target gene recited in Table 4. In an embodiment, the present specification provides for, and includes, a host cell transformed with a recombinant vector comprising a promoter polynucleotide sequence of SEQ ID NO:6 functionally linked to a heterologous target gene recited in Table 4. In an embodiment, the present specification provides for, and includes, a host cell transformed with a recombinant vector comprising a promoter polynucleotide sequence of SEQ ID NO:7 functionally linked to a heterologous target gene recited in Table 4. In an embodiment, the present specification provides for, and includes, a host cell transformed with a recombinant vector comprising a promoter polynucleotide sequence of SEQ ID NO:8 functionally linked to a heterologous target gene recited in Table 4.

The present specification provides for a host cell transformed with a recombinant vector comprising a promoter polynucleotide sequence selected from the group consisting of SEQ ID NOs:1 to 8 functionally linked to any one of the off-pathway heterologous target genes from Corynebacterium glutamicum ATCC 13032 provided in Table 5 or their Corynebacterium glutamicum equivalent thereof. Sequence start and end positions correspond to genomic nucleotide accession NC 003450.3. It will be understood by those of ordinary skill in the art that corresponding genes exist in other strains of C. glutamicum and may be readily identified from Table 5. In an embodiment, the present specification provides for, and includes, a host cell transformed with a recombinant vector comprising a promoter polynucleotide sequence of SEQ ID NO:1 functionally linked to a heterologous target gene recited in Table 5. In an embodiment, the present specification provides for, and includes, a host cell transformed with a recombinant vector comprising a promoter polynucleotide sequence of SEQ ID NO:2 functionally linked to a heterologous target gene recited in Table 5. In an embodiment, the present specification provides for, and includes, a host cell transformed with a recombinant vector comprising a promoter polynucleotide sequence of SEQ ID NO:3 functionally linked to a heterologous target gene recited in Table 5. In an embodiment, the present specification provides for, and includes, a host cell transformed with a recombinant vector comprising a promoter polynucleotide sequence of SEQ ID NO:4 functionally linked to a heterologous target gene recited in Table 5. In an embodiment, the present specification provides for, and includes, a host cell transformed with a recombinant vector comprising a promoter polynucleotide sequence of SEQ ID NO:5 functionally linked to a heterologous target gene recited in Table 5. In an embodiment, the present specification provides for, and includes, a host cell transformed with a recombinant vector comprising a promoter polynucleotide sequence of SEQ ID NO:6 functionally linked to a heterologous target gene recited in Table 5. In an embodiment, the present specification provides for, and includes, a host cell transformed with a recombinant vector comprising a promoter polynucleotide sequence of SEQ ID NO:7 functionally linked to a heterologous target gene recited in Table 5. In an embodiment, the present specification provides for, and includes, a host cell transformed with a recombinant vector comprising a promoter polynucleotide sequence of SEQ ID NO:8 functionally linked to a heterologous target gene recited in Table 5.

TABLE 5
C. glutamicum Off-Pathway Target Genes

| | | C. glutamicum | |
|---|---|---|---|
| Symbol | Gene Name (EC #) | Gene | Position |
| | Putative | NCgl0019 | 21501 . . . 21872 |
| | uncharacterized | | |
| | protein Cgl0020 | | |
| | Rhodanese-related | NCgl0054 | complement |
| | sulfurtransferases | | (92527 . . . 93120) |
| ureR | Transcriptional | NCgl0082 | complement |
| | regulators | | (128299 . . . 128814) |
| tyrA | Prephenate | NCgl0223 | complement |
| | dehydrogenase | | (283783 . . . 284805) |
| | [EC 1.3.1.12] | | |
| topA | DNA | NCgl0304 | 375048 . . . 378053 |
| | topoisomerase 1 | | |
| | [EC 5.99.1.2] | | |
| nusG | Transcription | NCgl0458 | 604434 . . . 605405 |
| | termination/ | | |
| | antitermination | | |
| | protein NusG | | |
| rpoB | DNA-directed RNA | NCgl0471 | 619984 . . . 623463 |
| | polymerase subunit | | |
| | beta (RNAP | | |
| | subunit beta) | | |
| | [EC 2.7.7.6] | | |
| | Transcriptional | NCgl0601 | 758218 . . . 758801 |
| | regulators | | |
| rhlE | Superfamily II DNA | NCgl0737 | complement |
| | and RNA helicases | | (925165 . . . 926439) |
| prfB | Peptide chain | NCgl0767 | 962986 . . . 964092 |
| | release factor 2 | | |
| | (RF-2) | | |
| purH | Bifunctional purine | NCgl0827 | 1042072 . . . 1043634 |
| | biosynthesis | | |
| | protein PurH | | |
| | [EC 2.1.2.3], | | |
| | IMP cyclohydrolase | | |
| | [EC 3.5.4.10] | | |
| | Putative | NCgl0966 | 1184332 . . . 1185576 |
| | uncharacterized | | |
| | protein Cgl1009 | | |
| rho | Transcription | NCgl1152 | 1386927 . . . 1389359 |
| | termination | | |
| | factor Rho | | |
| | [EC 3.6.4.—] | | |
| | Transcriptional | NCgl1261 | complement |
| | regulator | | (1533093 . . . 1533800) |
| leuC | 3-isopropylmalate | NCgl1262 | 1534054 . . . 1535493 |
| | dehydratase large | | |
| | subunit | | |
| | [EC 4.2.1.33] | | |
| ddl | D-alanine-- | NCgl1267 | 1538769 . . . 1539844 |
| | D-alanine | | |
| | ligase | | |
| | [EC 6.3.2.4] | | |
| | Predicted | NCgl1301 | 1571632 . . . 1572609 |
| | transcriptional | | |
| | regulators | | |
| cmk | Cytidylate kinase | NCgl1372 | 1667477 . . . 1668187 |
| | (CK) | | |
| | [EC 2.7.4.25] | | |
| ctaB | Protoheme IX | NCgl1511 | complement |
| | farnesyltransferase | | (1826368 . . . 1827339) |
| | [EC 2.5.1.—] | | |
| | Transcriptional | NCgl2425 | complement |
| | regulators | | (2640625 . . . 2641119) |
| | tRNA- | NCgl2481 | 2699108 . . . 2700253 |
| | dihydrouridine | | |
| | synthase | | |
| | [EC 1.3.1.—] | | |
| | Transcriptional | NCgl2527 | complement |
| | regulator | | (2760930 . . . 2761629) |
| | AraC-type | NCgl2587 | 2824273 . . . 2825286 |
| | DNA-binding | | |
| | domain-containing | | |
| | proteins | | |
| hspR | Predicted | NCgl2699 | complement |
| | transcriptional | | (2955572 . . . 2956012) |
| | regulators | | |
| dnaK | Chaperone protein | NCgl2702 | complement |
| | DnaK (HSP70) | | (2958093 . . . 2959949) |
| | Transcriptional | NCgl2802 | 3083687 . . . 3084724 |
| | regulator | | |
| mutM2 | Formamido- | NCgl2898 | 3217037 . . . 3217852 |
| | pyrimidine- | | |
| | DNA glycosylase | | |
| | [EC 3.2.2.23] | | |
| | Transcriptional | NCgl2921 | complement |
| | regulator | | (3246164 . . . 3246940) |
| | Uncharacterized | NCgl2982 | 3305877 . . . 3309221 |
| | membrane protein, | | |
| | putative virulence | | |
| | factor | | |

In an embodiment, the present specification provides for, and includes a recombinant vector comprising a promoter polynucleotide sequence of SEQ ID NO:3 functionally linked to an off-pathway heterologous target gene recited in Table 5. In an embodiment, the present specification provides for, and includes a recombinant vector comprising a promoter polynucleotide sequence of SEQ ID NO:4 functionally linked to a heterologous target gene recited in Table 5. In an embodiment, the present specification provides for, and includes a recombinant vector comprising a promoter polynucleotide sequence of SEQ ID NO:5 functionally linked to a heterologous target gene recited in Table 5. In an embodiment, the present specification provides for, and includes a recombinant vector comprising a promoter polynucleotide sequence of SEQ ID NO:6 functionally linked to a heterologous target gene recited in Table 5. In an embodiment, the present specification provides for, and includes a recombinant vector comprising a promoter polynucleotide sequence of SEQ ID NO:7 functionally linked to a heterologous target gene recited in Table 5. In an embodiment, the present specification provides for, and includes a recombinant vector comprising a promoter polynucleotide sequence of SEQ ID NO:8 functionally linked to a heterologous target gene recited in Table 5.

In an embodiment, the present specification provides for, and includes, a host cell transformed with a recombinant vector comprising a promoter polynucleotide sequence of SEQ ID NO:1 functionally linked to an off-pathway heterologous target gene recited in Table 5. In an embodiment, the present specification provides for, and includes, a host cell transformed with a recombinant vector comprising a promoter polynucleotide sequence of SEQ ID NO:2 functionally linked to a heterologous target gene recited in Table 5. In an embodiment, the present specification provides for, and includes, a host cell transformed with a recombinant vector comprising a promoter polynucleotide sequence of SEQ ID NO:3 functionally linked to a heterologous target gene recited in Table 5. In an embodiment, the present specification provides for, and includes, a host cell transformed with a recombinant vector comprising a promoter polynucleotide sequence of SEQ ID NO:4 functionally linked to a heterologous target gene recited in Table 5. In an embodiment, the present specification provides for, and includes, a host cell transformed with a recombinant vector comprising a promoter polynucleotide sequence of SEQ ID NO:5 functionally linked to a heterologous target gene recited in Table 5. In an embodiment, the present specification provides for, and includes, a host cell transformed with a recombinant vector comprising a promoter polynucleotide sequence of SEQ ID NO:6 functionally linked to a heterologous target gene recited in Table 5. In an embodiment, the present specification provides for, and includes, a host cell transformed with a recombinant vector comprising a promoter polynucleotide sequence of SEQ ID NO:7 functionally linked to a heterologous target gene recited in Table 5. In an embodiment, the present specification provides for, and includes, a host cell transformed with a recombinant vector comprising a promoter polynucleotide sequence of SEQ ID NO:8 functionally linked to a heterologous target gene recited in Table 5.

In an embodiment, the present specification provides for, and includes a recombinant vector comprising a promoter polynucleotide sequence of SEQ ID NO:1 functionally linked to an off-pathway heterologous target gene recited in Table 10. In an embodiment, the present specification provides for, and includes a recombinant vector comprising a promoter polynucleotide sequence of SEQ ID NO:2 functionally linked to an off-pathway heterologous target gene recited in Table 10. In an embodiment, the present specification provides for, and includes a recombinant vector comprising a promoter polynucleotide sequence of SEQ ID NO:3 functionally linked to an off-pathway heterologous target gene recited in Table 10. In an embodiment, the present specification provides for, and includes a recombinant vector comprising a promoter polynucleotide sequence of SEQ ID NO:4 functionally linked to a heterologous target gene recited in Table 10. In an embodiment, the present specification provides for, and includes a recombinant vector comprising a promoter polynucleotide sequence of SEQ ID NO:5 functionally linked to a heterologous target gene recited in Table 10. In an embodiment, the present specification provides for, and includes a recombinant vector comprising a promoter polynucleotide sequence of SEQ ID NO:6 functionally linked to a heterologous target gene recited in Table 10. In an embodiment, the present specification provides for, and includes a recombinant vector comprising a promoter polynucleotide sequence of SEQ ID NO:7 functionally linked to a heterologous target gene recited in Table 10. In an embodiment, the present specification provides for, and includes a recombinant vector comprising a promoter polynucleotide sequence of SEQ ID NO:8 functionally linked to a heterologous target gene recited in Table 10.

The present specification provides for a host cell transformed with a recombinant vector comprising a promoter polynucleotide sequence selected from a plurality of promoter polynucleotides comprising a promoter ladder. In some cases, the host cell is a component of a plurality of transformed host cells comprising the promoter ladder, e.g., wherein each cell of the plurality comprises a different promoter polynucleotide of the promoter ladder. In some cases, the promoter polynucleotides of the promoter ladder, in the same or different transformed host cells of the plurality, are operably linked to the same heterologous, e.g., ancillary, target gene. In some cases, the heterologous target gene is a shell 2, a shell 3, and/or a shell 4 heterologous target gene. In some cases, the heterologous target gene is a shell 3, and/or a shell 4 heterologous target gene. In some cases the heterologous target gene is a shell 4 heterologous target gene. In some cases, the heterologous target gene is a shell 2 heterolgous target gene. In some cases, the heterologous target gene is a shell 3 heterologous target gene. In some cases, the heterologous target gene is a heterologous target gene from Corynebacterium glutamicum, such as the heterologous target genes provided in Table 10, or optionally any one of the tables described herein. Sequence start and end positions in Table 10 correspond to genomic nucleotide accession NC_003450.3. It will be understood by those of ordinary skill in the art that corresponding genes exist in other strains of C. glutamicum and may be readily identified from the present disclosure.

In some cases, the promoter polynucleotides comprising the promoter ladder are selected from the group consisting of SEQ ID NOs:1 to 8 functionally linked to an off-pathway heterologous target gene, e.g., a shell 2, a shell 3, and/or a shell 4 heterologous target gene, an off-pathway heterologous target gene provided in Table 10, or optionally an off pathway target gene in any one of the tables described herein. In some cases the heterologous target gene is a shell 4 heterologous target gene.

In an embodiment, the present specification provides for, and includes, a host cell transformed with a recombinant vector comprising a promoter polynucleotide sequence of SEQ ID NO:1 functionally linked to a shell 2, a shell 3, and/or a shell 4 heterologous target gene, e.g., a heterologous target gene recited in Table 10. In some cases the heterologous target gene is a shell 4 heterologous target gene. In an embodiment, the present specification provides for, and includes, a host cell transformed with a recombinant vector comprising a promoter polynucleotide sequence of SEQ ID NO:2 functionally linked to a shell 2, a shell 3, and/or a shell 4 heterologous target gene, e.g., a heterologous target gene recited in Table 10. In some cases the heterologous target gene is a shell 4 heterologous target gene. In an embodiment, the present specification provides for, and includes, a host cell transformed with a recombinant vector comprising a promoter polynucleotide sequence of SEQ ID NO:3 functionally linked to a shell 2, a shell 3, and/or a shell 4 heterologous target gene, e.g., a heterologous target gene recited in Table 10. In some cases the heterologous target gene is a shell 4 heterologous target gene. In an embodiment, the present specification provides for, and includes, a host cell transformed with a recombinant vector comprising a promoter polynucleotide sequence of SEQ ID NO:4 functionally linked to a shell 2, a shell 3, and/or a shell 4 heterologous target gene, e.g., a heterologous target gene recited in Table 10. In some cases the heterologous target gene is a shell 4 heterologous target gene.

In an embodiment, the present specification provides for, and includes, a host cell transformed with a recombinant vector comprising a promoter polynucleotide sequence of SEQ ID NO:5 functionally linked to a shell 2, a shell 3, and/or a shell 4 heterologous target gene, e.g., a heterologous target gene recited in Table 10. In some cases the heterologous target gene is a shell 4 heterologous target gene. In an embodiment, the present specification provides for, and includes, a host cell transformed with a recombinant vector comprising a promoter polynucleotide sequence of SEQ ID NO:6 functionally linked to a shell 2, a shell 3, and/or a shell 4 heterologous target gene, e.g., a heterologous target gene recited in Table 10. In some cases the heterologous target gene is a shell 4 heterologous target gene.

In an embodiment, the present specification provides for, and includes, a host cell transformed with a recombinant vector comprising a promoter polynucleotide sequence of SEQ ID NO:7 functionally linked to a shell 2, a shell 3, and/or a shell 4 heterologous target gene, e.g., a heterologous target gene recited in Table 10. In some cases the heterologous target gene is a shell 2 heterologous target gene. In an embodiment, the present specification provides for, and includes, a host cell transformed with a recombinant vector comprising a promoter polynucleotide sequence of SEQ ID NO:8 functionally linked to a functionally linked to a shell 2, a shell 3, and/or a shell 4 heterologous target gene, e.g., a heterologous target gene recited in Table 10. In some cases the heterologous target gene is a shell 4 heterologous target gene.

In an embodiment, the present specification provides for, and includes a recombinant vector comprising a promoter polynucleotide sequence of SEQ ID NO:3 functionally linked to an off-pathway heterologous target gene recited in Table 10. In an embodiment, the present specification provides for, and includes a recombinant vector comprising a promoter polynucleotide sequence of SEQ ID NO:4 functionally linked to a heterologous target gene recited in Table 10. In an embodiment, the present specification provides for, and includes a recombinant vector comprising a promoter polynucleotide sequence of SEQ ID NO:5 functionally linked to a heterologous target gene recited in Table 10. In an embodiment, the present specification provides for, and includes a recombinant vector comprising a promoter polynucleotide sequence of SEQ ID NO:6 functionally linked to a heterologous target gene recited in Table 10. In an embodiment, the present specification provides for, and includes a recombinant vector comprising a promoter polynucleotide sequence of SEQ ID NO:7 functionally linked to a heterologous target gene recited in Table 10. In an embodiment, the present specification provides for, and includes a recombinant vector comprising a promoter polynucleotide sequence of SEQ ID NO:8 functionally linked to a heterologous target gene recited in Table 10.

In an embodiment, the present specification provides for, and includes, a host cell transformed with a recombinant vector comprising a promoter polynucleotide sequence of SEQ ID NO:1 functionally linked to an off-pathway heterologous target gene recited in Table 10. In an embodiment, the present specification provides for, and includes, a host cell transformed with a recombinant vector comprising a promoter polynucleotide sequence of SEQ ID NO:2 functionally linked to a heterologous target gene recited in Table 10. In an embodiment, the present specification provides for, and includes, a host cell transformed with a recombinant vector comprising a promoter polynucleotide sequence of SEQ ID NO:3 functionally linked to a heterologous target gene recited in Table 10. In an embodiment, the present specification provides for, and includes, a host cell transformed with a recombinant vector comprising a promoter polynucleotide sequence of SEQ ID NO:4 functionally linked to a heterologous target gene recited in Table 10. In an embodiment, the present specification provides for, and includes, a host cell transformed with a recombinant vector comprising a promoter polynucleotide sequence of SEQ ID NO:5 functionally linked to a heterologous target gene recited in Table 10. In an embodiment, the present specification provides for, and includes, a host cell transformed with a recombinant vector comprising a promoter polynucleotide sequence of SEQ ID NO:6 functionally linked to a heterologous target gene recited in Table 10. In an embodiment, the present specification provides for, and includes, a host cell transformed with a recombinant vector comprising a promoter polynucleotide sequence of SEQ ID NO:7 functionally linked to a heterologous target gene recited in Table 10. In an embodiment, the present specification provides for, and includes, a host cell transformed with a recombinant vector comprising a promoter polynucleotide sequence of SEQ ID NO:8 functionally linked to a heterologous target gene recited in Table 10.

As used herein, a host cell refers to an organisms described below in the section entitled ‘Expression’ that have been transformed with one or more of the promoter cassettes. As will be apparent to one of ordinary skill in the art, a host cell may comprise one or more promoter cassettes as described herein.

In some embodiments, the target gene is associated with a biosynthetic pathway producing a biomolecule selected from: amino acids, organic acids, flavors and fragrances, biofuels, proteins and enzymes, polymers/monomers and other biomaterials, lipids, nucleic acids, small molecule therapeutics, protein therapeutics, fine chemicals, and nutraceuticals.

In some embodiments the target gene is associated with a biosynthetic pathway producing a secondary metabolite selected from: antibiotics, alkaloids, terpenoids, and polyketides. In some embodiments the target gene is associated with a metabolic pathway producing a primary metabolite selected from: alcohols, amino acids, nucleotides, antioxidants, organic acids, polyols, vitamins, and lipids/fatty acids. In some embodiments the target gene is associated with a biosynthetic pathway producing a macromolecule selected from: proteins, nucleic acids, and polymers

In addition it may be advantageous for the production of L-amino acids to enhance, in particular to overexpress one or more enzymes of the respective biosynthesis pathway, glycolysis, anaplerosis, citric acid cycle, pentose phosphate cycle, amino acid export and optionally regulatory proteins.

Thus for example, for the production of L-amino acids, it may be advantageous for one or more genes selected from the following group to be enhanced, in particular overexpressed: the gene dapA coding for dihydrodipicolinate synthase (EP-B 0 197 335); the gene eno coding for enolase (DE: 19947791.4); the gene gap coding for glyceraldehyde-3-phosphate dehydrogenase (Eikmanns (1992), Journal of Bacteriology 174:6076-6086); the gene tpi coding for triosephosphate isomerase (Eikmanns (1992), Journal of Bacteriology 174:6076-6086); the gene pgk coding for 3-phosphoglycerate kinase (Eikmanns (1992), Journal of Bacteriology 174:6076-6086); the gene zwf coding for glucose-6-phosphate dehydrogenase (JP-A-09224661); the gene pyc coding for pyruvate carboxylase (DE-A-198 31 609; Eikmanns (1992), Journal of Bacteriology 174:6076-6086); the gene mqo coding for malate-quinone-oxidoreductase (Molenaar et al., European Journal of Biochemistry 254, 395-403 (1998)); the gene lysC coding for a feedback-resistant aspartate kinase (Accession No. P26512); the gene lysE coding for lysine export (DE-A-195 48 222); the gene hom coding for homoserine dehydrogenase (EP-A 0131171); the gene ilvA coding for threonine dehydratase (Mockel et al., Journal of Bacteriology (1992) 8065-8072)) or the allele ilvA (Fbr) coding for a feedback-resistant threonine dehydratase (Mockel et al., (1994) Molecular Microbiology 13: 833-842); the gene ilvBN coding for acetohydroxy acid synthase (EP-B 0356739); the gene ilvD coding for dihydroxy acid dehydratase (Sahm and Eggeling (1999) Applied and Environmental Microbiology 65: 1973-1979); and the gene zwa1 coding for the Zwa1 protein (DE: 19959328.0, DSM 13115).

Furthermore it may be advantageous for the production of L-amino acids also to attenuate, in particular to reduce, the expression of one or more genes selected from the group: the gene pck coding for phosphoenol pyruvate carboxykinase (DE 199 50 409.1; DSM 13047); the gene pgi coding for glucose-6-phosphate isomerase (U.S. Pat. No. 6,586,214; DSM 12969); the gene poxB coding for pyruvate oxidase (DE: 1995 1975.7; DSM 13114); and the gene zwa2 coding for the Zwa2 protein (DE: 19959327.2, DSM 13113).

In addition, it may furthermore be advantageous, for the production of amino acids, in particular L-lysine, to eliminate undesirable side reactions, (Nakayama: “Breeding of Amino Acid Producing Micro-organisms”, in: Overproduction of Microbial Products, Krumphanzl, Sikyta, Vanek (eds.), Academic Press, London, U K, 1982).

The promoter according to the disclosure can thus be used in each case for overexpressing or underexpressing the target gene in C. glutamicum.

### Methods of Identifying Ancillary Target Genes for Optimizing Production of Target Biomolecules

Described herein are methods for screening for and/or identifying ancillary target genes for modulation of expression and/or activity to improve target biomolecule production. In some cases, the heterologous ancillary target genes are shell 2, shell 3, and/or shell 4 target genes. In some cases, the heterologous ancillary target genes are shell 3 and/or shell 4 target genes. In some cases, the heterologous ancillary target genes are shell 4 target genes. Typically the methods involve screening a library of transformed host cells, wherein individual transformed host cells of the library comprise a different [promoter polynucleotide: operably linked heterologous ancillary target gene] combination as compared to other transformed cells of the library. Such combinations can then be identified from the library that improve target biomolecule production and used for manufacture of target biomolecule or further optimized.

Thus the methods can include one or more steps of providing such a library, and/or screening such a library, and/or identifying transformants exhibiting improved target molecule production, and/or isolating such improved transformants, and/or storing or expanding such improved transformants. In some embodiments, the promoter polynucleotides comprise a promoter ladder.

Generally, transformed host cells of the library further comprise an on-pathway modification. In some cases, the on-pathway modification is the same for all, essentially all, substantially all, or a majority of the transformed cells of the library. For example, for lysine production, all, essentially all, substantially all, or a majority of the transformed cells of the library can comprise a promoter polynucleotide operably linked to the on-pathway heterologous target gene lysA and/or one or more other promoter polynucleotide(s) operably linked to on-pathway heterologous target gene(s). In some cases, the transformed host cells comprise a wild-type strain background such that endogenous on-pathway target genes are operably linked to their corresponding endogenous promoters.

The library of transformed cells can comprise a promoter ladder, wherein the individual promoter polynucleotides of the promoter ladder are in different cells of the library. In general, different promoter polynucleotides of the promoter ladder are operably linked to the same heterologous ancillary target gene in the different transformed cells. As an example, for a library comprising a promoter ladder having eight different promoter polynucleotides and interrogating a single heterologous ancillary target gene, the minimum library size is eight cells, one cell containing each possible [promoter polynucleotide: operably linked heterologous ancillary target gene] combination, or nine cells where one cell is a control cell without a promoter polynucleotide of the promoter ladder. One of skill the in art can appreciate that the library of transformed host cells can contain a plurality (e.g., >10; >100; >1,000; 10-1,000; 10-10,000; or 100-100,000) of redundant copies of the minimal cellular set, of the library or a subset thereof. The library can further comprise an additional set of cells for each interrogated heterologous ancillary target gene, such that each interrogated heterologous ancillary target gene is operably linked to each of the different promoter polynucleotides of the promoter ladder in a different cell. This provides a set of cells, where each cell in the library is an experiment interrogating a different [promoter polynucleotide: operably linked heterologous ancillary target gene] combination.

The library can be provided by a number of techniques available to one of skill in the art. For example, a plurality of host cells having a selected background (e.g., modified for lysA overexpression) can be transformed with a library of recombinant vectors under conditions such that substantially all transformants are singly modified to contain a single [promoter polynucleotide operably linked heterologous ancillary target gene] combination. The recombinant vectors can be integrating vectors, such that the providing comprises engineering the genome of the host cell. The transformants can be isolated, stored, and/or expanded, and optionally assayed for target molecule production. Exemplary isolating methods include without limitation limiting dilution, plating, streaking, and/or colony picking. Exemplary storage methods include without limitation cryopreservation or sporulation. For example, transformants can be isolated, mixed with a suitable cryoprotectant (e.g., glycerol), cryogenically frozen under conditions suitable to limit ice crystal formation, and stored.

Moreover, the interrogated heterologous ancillary target genes can be assayed in plurality of (e.g., two or more) different on-pathway modification backgrounds. The assay of different on-pathway backgrounds can be performed simultaneously, e.g., in parallel, or sequentially. For example, the library of transformed host cells for increasing production of lysine can comprise a first sub-library of transformed host cells having a lysA overexpression modification and interrogating a plurality of [promoter polynucleotide operably linked heterologous target gene] combinations; and a second sub-library that differs from the first sub-library by having a different, or additional, on-pathway modification. Similarly, the library can comprise, or further comprise an off-pathway modification background and interrogating a plurality of [promoter polynucleotide operably linked heterologous target gene] combinations and/or interrogating a plurality of [promoter polynucleotide operably linked heterologous ancillary target gene] combinations. As an example, a library of transformed host cells for increasing production of lysine can comprise transformed host cells having a background comprising: an on-pathway lysA overexpression modification; an off-pathway pgi overexpression modification; and various [promoter polynucleotide operably linked heterologous ancillary target gene] combinations.

In some embodiments, the method includes identifying a host cell from the plurality of host cells that exhibits increased production of the target biomolecule. In some cases, the identifying step includes a reproducibility filter to identify host cells, and the underlying [promoter polynucleotide operably linked heterologous ancillary target gene] combinations that reproducibly exhibit increased production of the target biomolecule. For example, the identifying step can assay redundant copies of each [promoter polynucleotide operably linked heterologous ancillary target gene] combination and identify combinations that exhibit reproducibly improved target biomolecule production in all, substantially all, or a majority of the redundant copies. As another example, a statistical filter can be applied to exclude combinations that do not meet a selected level of statistical significance (e.g., p<0.05, 0.01, 0.005, or 0.001).

In some embodiments, the method can comprise an iterative method of providing a library. For example, a library can be provided, cultured, and one or more host cells exhibiting increased production of target biomolecule can comprise the background strain for a second round of library generation and screening. Thus, in some embodiments, a subsequent iteration creates a new host cell library comprising individual host cells harboring unique genetic variations that are a combination of genetic variation selected from amongst at least two individual host cells of a preceding host cell library. Iterations can be performed multiple times until a resulting host cell has acquired a selected level of target biomolecule production improvement; until further rounds of providing and screening a library exhibit diminishing improvement; or until improvement pleateus. In an embodiment, at least one round interrogates heterologous ancillary target genes. Additionally or alternatively, on-pathway genes can be interrogated in earlier or later rounds of library generation and screening, optionally in combination with further interrogation of heterologous ancillary target genes.

### Linkers

The target gene is positioned downstream of the promoter polynucleotide according to the invention, i.e. at the 3′ end, such that both polynucleotides are functionally linked to one another either directly or by means of a linker oligonucleotide or linker polynucleotide. Preference is given to the promoter and the target gene being functionally linked to one another by means of a linker oligonucleotide or linker polynucleotide. Said linker oligonucleotide or linker polynucleotide consists of deoxyribonucleotides.

In this context, the expression “functionally linked to one another directly” means that the nucleotide at the 3′ end of the promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, is linked directly to the first nucleotide of the start codon of a target gene. This results in “leaderless” mRNAs which start immediately with the 5′-terminal AUG start codon and therefore do not have any other translation initiation signals.

In this context, the expression “functionally linked to one another by means of a linker oligonucleotide” means that the nucleotide at the 3′ end of the promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, is linked by an oligonucleotide of 1, 2, 3, 4 or 5 nucleotides in length to the first nucleotide of the start codon of a target gene.

In this context, the expression “functionally linked to one another by means of a linker polynucleotide” means that the nucleotide at the 3′ end of the promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, is linked by a polynucleotide of from 6 to no more than 600 nucleotides in length to the first nucleotide of the start codon of a target gene.

In this context, the expression “functionally linked to one another” means that the target gene is bound to the promoter polynucleotide according to the invention in such a way that transcription of the target gene and translation of the synthesized RNA are ensured.

Depending on the technical requirement, the linker polynucleotide is:

6-600, 6-500, 6-400, 6-300, 6-200, 6-180, 6-160, 6-140, 6-120, 6-100, 6-80, 6-60, 6-50, 6-40, 6-30, 6-28, 6-27, 6-26, or 6-25; or

8-600, 8-500, 8-400, 8-300, 8-200, 8-180, 8-160, 8-140, 8-120, 8-100, 8-80, 8-60, 8-50, 8-40, 8-30, 8-28, 8-27, 8-26, or 8-25; or

10-600, 10-500, 10-400, 10-300, 10-200, 10-180, 10-160, 10-140, 10-120, 10-100, 10-80, 10-60, 10-50, 10-40, 10-30, 10-28, 10-27, 10-26, or 10-25; or

12-600, 12-500, 12-400, 12-300, 12-200, 12-180, 12-160, 12-140, 12-120, 12-100, 12-80, 12-60, 12-50, 12-40, 12-30, 12-28, 12-27, 12-26, or 12-25; or

14-600, 14-500, 14-400, 14-300, 14-200, 14-180, 14-160, 14-140, 14-120, 14-100, 14-80, 14-60, 14-50, 14-40, 14-30, 14-28, 14-27, 14-26, or 14-20; or

16-600, 16-500, 16-400, 16-300, 16-200, 16-180, 16-160, 16-140, 16-120, 16-100, 16-80, 16-60, 16-50, 16-40, 16-30, 16-28, 16-27, 16-26, or 16-25; or

18-600, 18-500, 18-400, 18-300, 18-200, 18-180, 18-160, 18-140, 18-120, 18-100, 18-80, 18-60, 18-50, 18-40, 18-30, 18-28, 18-27, 18-26, or 18-25; or

20-600, 20-500, 20-400, 20-300, 20-200, 20-180, 20-160, 20-140, 20-120, 20-100, 20-80, 20-60, 20-50, 20-40, 20-30, 20-28, 20-27, 20-26, or 20-25 nucleotides in length.

In particularly preferred embodiments, the linker polynucleotide is 20, 21, 22, 23, 24, or 25 nucleotides in length because this produces preferably functional constructs.

The disclosure further relates accordingly to an isolated promoter polynucleotide, essentially consisting of a promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, which, via the nucleotide at its 3′ end, is functionally linked, directly or by means of a linker polynucleotide which ensures translation of RNA, to a target gene which contains at its 5′ end an ATG or GTG start codon and codes for one or more off-pathway polypeptide(s). Preference is given to the promoter and target gene being functionally linked to one another by means of a linker polynucleotide.

The disclosure furthermore also relates to an isolated polynucleotide, essentially consisting of a promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, which, via the nucleotide at its 3′ end, is functionally linked to a linker oligonucleotide.

In addition, the disclosure furthermore relates to an isolated polynucleotide, essentially consisting of a promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, which, via the nucleotide at its 3′ end, is functionally linked to a linker polynucleotide which ensures translation of RNA.

In this context, the term “essentially” means that a polynucleotide of no more than 1,000, no more than 800, no more than 700, no more than 600, no more than 500, or no more than 400 nucleotides in length has been added to the 5′ end of the promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, and a polynucleotide of no more than 1,000, no more than 800, no more than 700, no more than 600, no more than 500, or no more than 400 nucleotides in length has been added to the 3′ of the target gene, or a polynucleotide of no more than 15,000, no more than 10,000, no more than 7,500, no more than 5,000, no more than 2,500, no more than 1,000, no more than 800, no more than 700, no more than 600, no more than 500, or no more than 400 nucleotides in length has been added to the 3′ end of the linker oligo- or polynucleotide.

Any useful combination of the features from the preceding three lists of polynucleotides is in accordance with the invention here. “Useful combination” means, for example, a combination of features which results in an efficient recombination being carried out. The use of additions of the same length flanking a DNA region to be replaced facilitates the transfer of the region by homologous recombination in the experimental procedure. Relatively long flanking homologous regions are advantageous for efficient recombination between circular DNA molecules but cloning of the replacement vector is made more difficult with increasing length of the flanks (Wang et al., Molecular Biotechnology 32:43-53 (2006)).

In addition, the flank at the 3′ end of the linker oligo- or polynucleotide increases in length to no more than 15,000 nucleotides when the 3′ end is functionally linked to a target gene which contains at its 5′ end an ATG or GTG start codon and codes for one or more polypeptide(s).

These particularly preferred embodiments of the linker polynucleotide ensure translation of RNA in an advantageous manner.

To facilitate chemical linking between the promoter polynucleotide, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8, the linker polynucleotide which ensures translation of RNA, and the target gene coding for one or more polypeptide(s), which has an ATG or GTG start codon at its 5′ end, functional nucleotide sequences required for cloning may be incorporated into said polynucleotides at their 5′ and 3′ ends and are at least partially retained even after said cloning.

The term “functional nucleotide sequence required for cloning” here represents any REII (type II restriction endonuclease) cleavage site present, whose sequence normally consists of from 4 to 8 nucleotides.

In addition, it should be mentioned here that site-specific mutagenesis by means of mutagenesis primers or a de novo gene synthesis (e.g. by GENEART AG (Regensburg, Germany)) of the nucleotide sequences to remove cleavage sites for restriction endonucleases may introduce silent mutations into the sequence in order to enable said cleavage sites to be used advantageously for subsequent cloning steps.

The polynucleotide resulting from the promoter according to the invention being functionally linked to the linker polynucleotide which ensures translation of RNA is also referred to as expression unit herein below.

### Expression

The disclosure furthermore relates to the use of the promoter according to the invention or of the expression unit according to the invention for expressing target genes or polynucleotides in microorganisms. The promoter according to the invention or the expression unit according to the invention ensures transcription and translation of the synthesized RNA, preferably mRNA, into a polypeptide. As used herein, the term “host cell” refers to a transformed cell of a microorganism.

The present disclosure, provides for, and includes, transformed host cells comprising the recombinant nucleic acids and recombinant vectors described in detail above. The present disclosure further provides for, and includes, host cells transformed with two recombinant nucleic acids. In an embodiment, the host cells are transformed with three recombinant nucleic acids. As provided above, the nucleic acids may be selected from biosynthetic pathways based on the overall effect on the yield of the desired product. There is no practical limit the number of recombinant nucleic acids that may be incorporated into the host cells of the present specification. Expression is preferably carried out in microorganisms of the genus Corynebacterium. Preference is given to strains within the genus Corynebacterium which are based on the following species: C. efficiens, with the deposited type strain being DSM44549; C. glutamicum, with the deposited type strain being ATCC13032; and C. ammoniagenes, with the deposited type strain being ATCC6871. Very particular preference is given to the species C. glutamicum. In this way it is possible to express polynucleotides that code for polypeptides having a property, preferably enzyme activity, which are not present or detectable in the corresponding host. Thus, for example, Yukawa et al. describe expression of Escherichia coli genes for utilizing D-xylose in C. glutamicum R under the control of the constitutive Ptrc promoter (Applied Microbiology and Biotechnology 81, 691-699 (2008)).

The present specification provides for, and includes host cells such as C. glutamicum having two or more genes of a biosynthetic pathway under the control of the promoter polynucleotide sequences described above. In various embodiments, one or more target genes (e.g., ancillary target genes, and/or shell 2, and/or shell 3, and/or 4 target genes) are placed under the control of a promoter polynucleotide sequence having as sequence of SEQ ID NOs:1 to 8 as described above. In other embodiments, one or more target genes are placed under the control of a promoter polynucleotide sequence having as sequence of SEQ ID NOs:1, 5 or 7 as described above.

In certain embodiments according to the present specification, C. glutamicum host cells have two target genes under the control of the promoters having sequences of SEQ ID NOs:1 to 8. In certain other embodiments according to the present specification, C. glutamicum host cells have two target genes under the control of the promoters having sequences of SEQ ID NOs:1, 5 or 7. Using homologous recombination, the promoters of the present disclosure replace the endogenous promoter and endogenous sequence to prepare a promoter functionally linked to a heterologous gene. One of ordinary skill in the art would recognize that the recombination results in a replacement of the endogenous promoter while retaining the gene in its native locus. Specific non-limiting examples are illustrated below in Table 8. Multiple promoter-heterologous target pairs (e.g., promoter cassettes) can be readily incorporated into the genome of a host cell. In an embodiment, the promoter cassettes can be incorporated into host cells sequentially. In certain embodiments, the recombinant vectors of the present disclosure provide for two or more different promoter cassettes in a single construct. The present specification provides no practical limit to the number of promoter replacements that can be developed using the described methods.

Also described herein is a plurality of host cells comprising a promoter ladder, wherein one cell of the plurality comprises a first promoter polynucleotide operably linked to a heterologous target gene, e.g., an ancillary target gene, a shell 2 target gene, a shell 3 target gene, or a shell 4 target gene, and a second cell of the plurality comprises a second promoter polynucleotide operably linked to the same heterologous target gene, wherein the first and second promoter polynucleotides are different promoter polynucleotides of the promoter ladder.

In some cases, the plurality of host cells further comprise a third cell of the plurality comprising a third promoter polynucleotide operably linked to the same heterologous target gene, wherein the third promoter polynucleotide is a promoter polynucleotide of the promoter ladder that is different from the first and second promoter polynucleotides. In some cases, the plurality of host cells further comprise a fourth cell of the plurality comprising a fourth promoter polynucleotide operably linked to the same heterologous target gene, wherein the fourth promoter polynucleotide is a promoter polynucleotide of the promoter ladder that is different from the first, second, and third promoter polynucleotides. In some cases, the plurality of host cells further comprise a fifth cell of the plurality comprising a fifth promoter polynucleotide operably linked to the same heterologous target gene, wherein the fifth promoter polynucleotide is a promoter polynucleotide of the promoter ladder that is different from the first, second, third, and fourth promoter polynucleotides. In some cases, the plurality of host cells further comprise a sixth cell of the plurality comprising a sixth promoter polynucleotide operably linked to the same heterologous target gene, wherein the sixth promoter polynucleotide is a promoter polynucleotide of the promoter ladder that is different from the first, second, third, fourth, and fifth promoter polynucleotides. In some cases, the plurality of host cells further comprise a seventh cell of the plurality comprising a seventh promoter polynucleotide operably linked to the same heterologous target gene, wherein the seventh promoter polynucleotide is a promoter polynucleotide of the promoter ladder that is different from the first, second, third, fourth, fifth, and sixth promoter polynucleotides. In some cases, the plurality of host cells further comprise an eighth cell of the plurality comprising an eighth promoter polynucleotide operably linked to the same heterologous target gene, wherein the eighth promoter polynucleotide is a promoter polynucleotide of the promoter ladder that is different from the first, second, third, fourth, fifth, sixth, and seventh promoter polynucleotides.

In some cases each of the first, second, third, fourth, fifth, sixth, seventh, and/or eighth promoter polynucleotide of the promoter ladder is selected from SEQ ID NO:1-8. In some cases, the promoter polynucleotides of the promoter ladder are selected from SEQ ID NO:1, 5, and 7. The number of cells in the plurality can comprise at least about 1×10⁵, 1×10⁶, or 1×10⁷ cells.

In an embodiment the host cell is a transgenic C. glutamicum host cell comprising the promoter cassettes Pcg0007-lysA and Pcg3121-pgi. In an embodiment the host cell is a transgenic C. glutamicum host cell comprising the promoter cassettes Pcg1860-pyc and Pcg0007-zwf. In an embodiment the host cell is a transgenic C. glutamicum host cell comprising the promoter cassettes Pcg0007-lysA and Pcg0007-zwf. In an embodiment the host cell is a transgenic C. glutamicum host cell comprising the promoter cassettes Pcg3121-pck and Pcg0007-zwf. In an embodiment the host cell is a transgenic C. glutamicum host cell comprising the promoter cassettes Pcg0007_39-ppc and Pcg0007-zwf. In an embodiment the host cell is a transgenic C. glutamicum host cell comprising the promoter cassettes Pcg3121-pck and Pcg3121-pgi. In an embodiment the host cell is a transgenic C. glutamicum host cell comprising the promoter cassettes Pcg3381-ddh and Pcg0007-zwf. In an embodiment the host cell is a transgenic C. glutamicum host cell comprising the promoter cassettes Pcg0007_265-dapB and Pcg0007-zwf. In an embodiment the host cell is a transgenic C. glutamicum host cell comprising the promoter cassettes Pcg0007-zwf and Pcg3121-pgi. In an embodiment the host cell is a transgenic C. glutamicum host cell comprising the promoter cassettes Pcg3381-ddh and Pcg3121-pgi. In an embodiment the host cell is a transgenic C. glutamicum host cell comprising the promoter cassettes Pcg3121-pgi and Pcg1860-pyc. In an embodiment the host cell is a transgenic C. glutamicum host cell comprising the promoter cassettes Pcg1860-pyc and Pcg0007_265-dapB. In an embodiment the host cell is a transgenic C. glutamicum host cell comprising the promoter cassettes Pcg1860-pyc and Pcg0007-lysA. In an embodiment the host cell is a transgenic C. glutamicum host cell comprising the promoter cassettes Pcg1860-asd and Pcg0007-zwf. In an embodiment the host cell is a transgenic C. glutamicum host cell comprising the promoter cassettes Pcg0007_265-dapB and Pcg3121-pgi. In an embodiment the host cell is a transgenic C. glutamicum host cell comprising the promoter cassettes Pcg1860-pyc and Pcg1860-asd. In an embodiment the host cell is a transgenic C. glutamicum host cell comprising the promoter cassettes Pcg3381-aspB and Pcg1860-pyc. In an embodiment the host cell is a transgenic C. glutamicum host cell comprising the promoter cassettes Pcg3381-fbp and Pcg1860-pyc. In an embodiment the host cell is a transgenic C. glutamicum host cell comprising the promoter cassettes Pcg3381-ddh and Pcg3381-fbp. In an embodiment the host cell is a transgenic C. glutamicum host cell comprising the promoter cassettes Pcg0755-ptsG and Pcg3121-pgi. In an embodiment the host cell is a transgenic C. glutamicum host cell comprising the promoter cassettes Pcg1860-pyc and Pcg3121-pck. In an embodiment the host cell is a transgenic C. glutamicum host cell comprising the promoter cassettes Pcg1860-asd and Pcg3121-pgi. In an embodiment the host cell is a transgenic C. glutamicum host cell comprising the promoter cassettes Pcg1860-asd and Pcg33 81-fbp. In an embodiment the host cell is a transgenic C. glutamicum host cell comprising the promoter cassettes Pcg0007_39-lysE and Pcg3381-fbp. In an embodiment the host cell is a transgenic C. glutamicum host cell comprising the promoter cassettes Pcg3381-fbp and Pcg0007-lysA. In an embodiment the host cell is a transgenic C. glutamicum host cell comprising the promoter cassettes Pcg0007_39-lysE and Pcg1860-pyc. In an embodiment the host cell is a transgenic C. glutamicum host cell comprising the promoter cassettes Pcg3121-pgi and Pcg3381-fbp. In an embodiment the host cell is a transgenic C. glutamicum host cell comprising the promoter cassettes Pcg3121-pck and Pcg0007-lysA. In an embodiment the host cell is a transgenic C. glutamicum host cell comprising the promoter cassettes Pcg0007-lysA and Pcg0007_265-dapB. In an embodiment the host cell is a transgenic C. glutamicum host cell comprising the promoter cassettes Pcg0007_265-dapB and Pcg1860-asd. In an embodiment the host cell is a transgenic C. glutamicum host cell comprising the promoter cassettes Pcg3121-pgi and Pcg0007_265-dapD. In an embodiment the host cell is a transgenic C. glutamicum host cell comprising the promoter cassettes Pcg0007-lysA and Pcg3381-ddh. In an embodiment the host cell is a transgenic C. glutamicum host cell comprising the promoter cassettes Pcg3121-pck and Pcg1860-asd. In an embodiment the host cell is a transgenic C. glutamicum host cell comprising the promoter cassettes Pcg0007-lysA and Pcg1860-asd. In an embodiment the host cell is a transgenic C. glutamicum host cell comprising the promoter cassettes Pcg3121-pck and Pcg0007_265-dapB. In an embodiment the host cell is a transgenic C. glutamicum host cell comprising the promoter cassettes Pcg3381-ddh and Pcg1860-asd. In an embodiment the host cell is a transgenic C. glutamicum host cell comprising the promoter cassettes Pcg0007_39-ppc and Pcg1860-asd. In an embodiment the host cell is a transgenic C. glutamicum host cell comprising the promoter cassettes Pcg0007_39-ppc and Pcg0007-lysA. In an embodiment the host cell is a transgenic C. glutamicum host cell comprising the promoter cassettes Pcg3381-ddh and Pcg0007_265-dapB. In an embodiment the host cell is a transgenic C. glutamicum host cell comprising the promoter cassettes Pcg0007_265-dapB and Pcg3381-fbp. In an embodiment the host cell is a transgenic C. glutamicum host cell comprising the promoter cassettes Pcg0007_39-ppc and Pcg0007_265-dapB. In an embodiment the host cell is a transgenic C. glutamicum host cell comprising the promoter cassettes Pcg3381-aspB and Pcg3121-pck. In an embodiment the host cell is a transgenic C. glutamicum host cell comprising the promoter cassettes Pcg0007_265-dapB and Pcg0007_265-dapD. In an embodiment the host cell is a transgenic C. glutamicum host cell comprising the promoter cassettes Pcg0007_39-lysE and Pcg3381-aspB. In an embodiment the host cell is a transgenic C. glutamicum host cell comprising the promoter cassettes Pcg0007_39-lysE and Pcg0007_265-dapD. In an embodiment the host cell is a transgenic C. glutamicum host cell comprising the promoter cassettes Pcg3381-aspB and Pcg0007_265-dapB. In an embodiment the host cell is a transgenic C. glutamicum host cell comprising the promoter cassettes Pcg1860-asd and Pcg0007_265-dapD. In an embodiment the host cell is a transgenic C. glutamicum host cell comprising the promoter cassettes Pcg3381-aspB and Pcg0007-lysA. In an embodiment the host cell is a transgenic C. glutamicum host cell comprising the promoter cassettes Pcg3381-aspB and Pcg3381-ddh. In an embodiment the host cell is a transgenic C. glutamicum host cell comprising the promoter cassettes Pcg0755-ptsG and Pcg1860-pyc. In an embodiment the host cell is a transgenic C. glutamicum host cell comprising the promoter cassettes Pcg0755-ptsG and Pcg3381-fbp. In an embodiment the host cell is a transgenic C. glutamicum host cell comprising the promoter cassettes Pcg0007-zwf and Pcg3381-fbp. In an embodiment the host cell is a transgenic C. glutamicum host cell comprising the promoter cassettes Pcg0755-ptsG and Pcg0007_265-dapD.

The present disclosure provides for, and includes, host cells having three or more promoter cassettes as described above. In an embodiment, the host cell includes the Pcg0007_39-zwf, Pcg0007_39-lysA and Pcg1860-pyc promoter cassettes. In an embodiment, the host cell is a C. glutamicum host cell.

In an embodiment, the host cell includes any one of the foregoing promoter cassettes, and/or includes pcg0007_39-dnak; pcg0007_39-cg0074; pcg3121-cg0074; pcg1860-rhle_609; pcg3121-cg1144; pcg1860-rhle_609; pcg0007_39-cg2899_2194; pcg0007_39-cg1486; pcg0007_39-cg2766; pcg0007_39-cmk; pcg0007_39-rpob_383; pcg0007_39-ddl; pcg0007_39-cg0027; pcg0007_39-ddl; pcg0007_39-rpob_383; pcg0007_39-rpob_383; pcg0007_39-cg0027; pcg1860-cg1144; pcg0007_39-cg0725; pcg0007_39-cg0027; pcg0007_39-cg1527; pcg0007_39-ddl; pcg0007_39-rpob_383; pcg0007_39-cg0725; pcg0007_39-cg0725; pcg0007_39-ddl; pcg0007_39-cg0725; pcg0007_39-cg2766; pcg0007_39-cg0725; pcg0007_39-hspr; pcg0007_39-cg3352; pcg0007_39-cg2899_2194; pcg0007_39-cg2766; pcg0007_39-cg2766; pcg0007_39-cg2965; pcg0007_39-rpob_383; pcg0007_39-cg2766; pcg0007_39-cg2766; pcg0007_39-cg2899_2194; pcg0007_39-cg0074; pcg3121-cg0074; pcg0007_39-cg2766; pcg3121-cg1144; pcg0007_39-cg2766; pcg0007_39-cg2766; pcg0007_39-cg2899; pcg0007_39-rho; pcg0007_39-cg2766; pcg0007_39-cg2766; pcg0007_39-cg0725; pcg1860-cg1144; pcg0007_39-cg2766 pcg0007_39-cg2766; pcg0007_39-urer; pcg0007_39-nusg; pcg3121-mutm2_2522; pcg0007_39-ddl; pcg1860-cg1144; pcg0007_39-cg2899; pcg0007_39-cg2965; pcg0007_39-ddl; pcg3121-mutm2_2522; pcg0007_39-cg2766; pcg0007_39-cg2766; pcg0007_39-cg2766; pcg0007_39-cg2766; pcg0007_39-cg2766; pcg0007_39-cg2766; pcg0007_39-tyra; pcg0007_39-cg1486; pcg0007_39-cg2899; pcg0007_39-cg0027; pcg0007_39-ncg11511; pcg0007_39-ncg11262; pcg0007_39-cg3419; pcg0007_39-cg1486; pcg0007_39-cg3210; pcg0007_39-cg1486; pcg0007_39-cg1486; pcg0007_39-cg1486; pcg0007_39-cg1486; pcg0007_39-ncg10767; pcg0007_39-ncg12481; pcg0007_39-tyra; pcg0007_39-cg1486; pcg0007_39-ncg11511; pcg0007_39-ncg10827; pcg0007_39-tyra; pcg0007_39-cg1486; pcg0007_39-ncg11262; pcg0007_39-cg1486; pcg0007_39-ncg11262; pcg0007_39-ncg11262; pcg0007_39-ncg10767; pcg0007_39-ncg10304; pcg0007_39-ncg11511; pcg0007_39-ncg10767; pcg0007_39-ncg11262; pcg0007_39-ncg11511; pcg0007_39-ncg10767; pcg0007_39-ncg10767; pcg0007_39-ncg11262; pcg0007_39-cg1486; pcg0007_39-ncg11262; pcg0007_39-ncg10304; pcg0007_39-ncg11262; pcg0007_39-ncg10767; pcg0007_39-ncg11262, or a combination of 1, 2, 3, 4, 5, 6, 7, 8, 9, 10, or all thereof.

The promoter according to the invention or the expression unit according to the invention is furthermore used for improving the performance characteristics of microorganisms, which can include, for example, yield, titer, productivity, by-product elimination, tolerance to process excursions, optimal growth temperature and growth rate. In some embodiments, the promoter according to the invention or the expression unit according to the invention is used for up-regulating a target gene in a microorganism (overexpression). Overexpression generally means an increase in the intracellular concentration or activity of a ribonucleic acid, a protein (polypeptide) or an enzyme in comparison with the starting strain (parent strain) or wild-type strain, if the latter is the starting strain. In some embodiments, the promoter according to the invention or the expression unit according to the invention is used for down-regulating a target gene in a microorganism (underexpression). Underexpression generally means an decrease in the intracellular concentration or activity of a ribonucleic acid, a protein (polypeptide) or an enzyme in comparison with the starting strain (parent strain) or wild-type strain, if the latter is the starting strain. In some embodiments, a combination of promoters and/or expression units according to the invention are used for regulating expression of more than one target gene in a microorganism, wherein each target gene is either up-regulated or down-regulated. In some embodiments the target genes up- or down-regulated by the combination of promoters and/or expression units are part of the same metabolic pathway. In some embodiments the target genes up- or down-regulated by the combination of promoters and/or expression units are not part of the same metabolic pathway.

The promoters described herein can be used in combination with other methods very well-known in the art for attenuating (reducing or eliminating) the intracellular activity of one or more enzymes (proteins) in a microorganism which are coded by the corresponding DNA, for example by using a weak promoter or using a gene, or allele, which codes for a corresponding enzyme with a low activity, or inactivates the corresponding gene or enzyme (protein), and optionally combining these measures.

The reduction in gene expression can take place by suitable culturing or by genetic modification (mutation) of the signal structures of gene expression. Signal structures of gene expression are, for example, repressor genes, activator genes, operators, promoters, attenuators, ribosome binding sites, the start codon and terminators. The expert can find information on this e.g. in the patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Patek et al. (Microbiology 142: 1297 (1996)), Vašicová et al. (Journal of Bacteriology 181: 6188 (1999)) and in known textbooks of genetics and molecular biology, such as e.g. the textbook by Knippers (“Molekulare Genetik [Molecular Genetics]”, 6th edition, Georg Thieme Verlag, Stuttgart, Germany, 1995) or that by Winnacker (“Gene und Klone [Genes and Clones]”, VCH Verlagsgesellschaft, Weinheim, Germany, 1990).

Mutations which lead to a change or reduction in the catalytic properties of enzyme proteins are known from the prior art; examples which may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation und Struktur des Enzyms [Threonine dehydratase from Corynebacterium glutamicum: Cancelling the allosteric regulation and structure of the enzyme]”, Reports from the Jülich Research Centre, Jül-2906, ISSN09442952, Jülich, Germany, 1994). Comprehensive descriptions can be found in known textbooks of genetics and molecular biology, such as e.g. that by Hagemann (“Allgemeine Genetik [General Genetics]”, Gustav Fischer Verlag, Stuttgart, 1986).

Possible mutations are transitions, transversions, insertions and deletions. Depending on the effect of the amino acid exchange on the enzyme activity, missense mutations or nonsense mutations are referred to. Insertions or deletions of at least one base pair in a gene lead to frame shift mutations, as a consequence of which incorrect amino acids are incorporated or translation is interrupted prematurely. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik [Molecular Genetics]”, 6th edition, Georg Thieme Verlag, Stuttgart, Germany, 1995), that by Winnacker (“Gene und Klone [Genes and Clones]”, VCH Verlagsgesellschaft, Weinheim, Germany, 1990) or that by Hagemann (“Allgemeine Genetik [General Genetics]”, Gustav Fischer Verlag, Stuttgart, 1986). A common method of mutating genes of C. glutamicum is the method of gene disruption and gene replacement described by Schwarzer and Pühler (Bio/Technology 9, 84-87 (1991)).

In the method of gene disruption a central part of the coding region of the gene of interest is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schafer et al., Gene 145, 69-73 (1994)), pK18mobsacB or pK19mobsacB (Jager et al., Journal of Bacteriology 174: 5462-65 (1992)), pGEM-T (Promega corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector which contains the central part of the coding region of the gene is then transferred into the desired strain of C. glutamicum by conjugation or transformation. The method of conjugation is described, for example, by Schafer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” event, the coding region of the gene in question is interrupted by the vector sequence and two incomplete alleles are obtained, one lacking the 3′ end and one lacking the 5′ end. This method has been used, for example, by Fitzpatrick et al. (Applied Microbiology and Biotechnology 42, 575-580 (1994)) to eliminate the recA gene of C. glutamicum.

In the method of gene replacement, a mutation, such as e.g. a deletion, insertion or base exchange, is established in vitro in the gene of interest. The allele prepared is in turn cloned in a vector which is not replicative for C. glutamicum and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch (Microbiology 144, 915-927 (1998)) to eliminate the pyc gene of C. glutamicum by a deletion.

The promoters described herein can be used in combination with other methods very well-known in the art for raising (enhancing) the intracellular activity of one or more enzymes in a microorganism that are coded by the corresponding DNA, by for example increasing the number of copies of the gene or genes, using a strong promoter, or using a gene that codes for a corresponding enzyme having a high activity, and optionally combining these measures.

In order to achieve an overexpression the number of copies of the corresponding genes can be increased, or alternatively the promoter and regulation region or the ribosome binding site located upstream of the structure gene can be mutated. Expression cassettes that are incorporated upstream of the structure gene act in the same way. By means of inducible promoters it is in addition possible to increase the expression in the course of the enzymatic amino acid production. The expression is similarly improved by measures aimed at prolonging the lifetime of the m-RNA. Furthermore, the enzyme activity is also enhanced by preventing the degradation of the enzyme protein. The genes or gene constructs may either be present in plasmids having different numbers of copies, or may be integrated and amplified in the chromosome. Alternatively, an overexpression of the relevant genes may furthermore be achieved by altering the composition of the media and the culture conditions.

The person skilled in the art can find details on the above in, inter alia, Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European Patent Specification 0 472 869, in U.S. Pat. No. 4,601,893, in Schwarzer and Pühler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in Patent Application WO 96/15246, in Malumbres et al. (Gene 134, 15-24 (1993)), in Japanese laid open Specification JP-A-10-229891, in Jensen and Hammer (Biotechnology and Bioengineering 58, 191-195 (1998)), in Makrides (Microbiological Reviews 60:512-538 (1996)) and in known textbooks on genetics and molecular biology.

Genes may be overexpressed for example by means of episomal plasmids. Suitable plasmids are those that are replicated in coryneform bacteria. Numerous known plasmid vectors, such as for example pZ1 (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)) are based on the cryptic plasmids pHM1519, pBL1 or pGA1. Other plasmid vectors, such as for example those based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891) may be used in a similar way.

Furthermore, also suitable are those plasmid vectors with the aid of which the process of gene amplification by integration in the chromosome can be employed, such as has been described for example by Reinscheid et al. (Applied and Environmental Microbiology 60, 126-132 (1994)) for the duplication and amplification of the hom-thrB operon. In this method the complete gene is cloned into a plasmid vector that can replicate in a host (typically E. coli) but not in C. glutamicum. Suitable vectors are for example pSUP301 (Simon et al., Bio/Technology 1, 784-791 (1983)), pK18mob or pK19 mob (Schafer et al., Gene 145, 69-73 (1994)), pGEM-T (Promega Corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Netherlands; Bernard et al., Journal of Molecular Biology, 234: 534-541 (1993)), pEM1 (Schrumpf et al, 1991, Journal of Bacteriology 173:4510-4516) or pBGS8 (Sprat et al., 1986, Gene 41: 337-342). The plasmid vector that contains the gene to be amplified is then transferred by conjugation or transformation into the desired strain of C. glutamicum. The method of conjugation is described for example in Schafer et al. (Applied and Environmental Microbiology 60, 756-759 (1994)). Transformation methods are described for example in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a crossover event, the resulting strain contains at least two copies of the relevant gene.

Methods of regulating, i.e., either increasing or decreasing, gene expression include recombinant methods in which a microorganism is produced using a DNA molecule provided in vitro. Such DNA molecules comprise, for example, promoters, expression cassettes, genes, alleles, coding regions, etc. They are introduced into the desired microorganisms by methods of transformation, conjugation, transduction or similar methods.

In the case of the present disclosure, the promoters are preferably a polynucleotide of SEQ ID NO:1, SEQ ID NO:2, SEQ ID NO:3, SEQ ID NO:4, SEQ ID NO:5, SEQ ID NO:6, SEQ ID NO:7, or SEQ ID NO:8, and the expression cassettes are preferably a polynucleotide of SEQ ID NO:1, SEQ ID NO:2, SEQ ID NO:3, SEQ ID NO:4, SEQ ID NO:5, SEQ ID NO:6, SEQ ID NO:7, or SEQ ID NO:8 which, via the nucleotide at its 3′ end, are functionally linked to a linker polynucleotide which ensures translation of RNA.

The measures of overexpression using the promoter according to the invention or the expression unit according to the invention increase the activity or concentration of the corresponding polypeptide usually by at least 10%, 25%, 50%, 75%, 100%, 150%, 200%, 300%, 400% or 500%, preferably by no more than 1,000%, 2,000%, 4,000%, 10,000% or 20,000%, based on the activity or concentration of said polypeptide in the strain prior to the measure resulting in overexpression.

The extent of expression or overexpression may be established by measuring the amount of mRNA transcribed from the gene, by determining the amount of polypeptide and by determining enzyme activity.

The amount of mRNA may be determined inter alia by using the methods of “Northern Blotting” and of quantitative RT-PCR. Quantitative RT-PCR involves reverse transcription which precedes the polymerase chain reaction. For this, the LightCycler™ System from Roche Diagnostics (Boehringer Mannheim GmbH, Roche Molecular Biochemicals, Mannheim, Germany) may be used, as described in Jungwirth et al. (FEMS Microbiology Letters 281, 190-197 (2008)), for example. The concentration of the protein may be determined via 1- and 2-dimensional protein gel fractionation and subsequent optical identification of the protein concentration using appropriate evaluation software in the gel. A customary method of preparing protein gels for coryneform bacteria and of identifying said proteins is the procedure described by Hermann et al. (Electrophoresis, 22:1712-23 (2001)). The protein concentration may likewise be determined by Western-Blot hybridization using an antibody specific for the protein to be detected (Sambrook et al., Molecular cloning: a laboratory manual. 2nd Ed. Cold Spring Harbor Laboratory Press, Cold Spring Harbor, N.Y., 1989) and subsequent optical evaluation using appropriate software for concentration determination (Lohaus and Meyer (1998) Biospektrum 5:32-39; Lottspeich, Angewandte Chemie 321: 2630-2647 (1999)). The statistical significance of the data collected is determined by means of a T test (Gosset, Biometrika 6(1): 1-25 (1908)).

The measure of overexpressing target genes using the promoter according to the invention may be combined in a suitable manner with further overexpression measures. Overexpression is achieved by a multiplicity of methods available in the prior art. These include increasing the copy number in addition to modifying the nucleotide sequences which direct or control expression of the gene. The copy number may be increased by means of plasmids which replicate in the cytoplasm of the microorganism. To this end, an abundance of plasmids are described in the prior art for very different groups of microorganisms, which plasmids can be used for setting the desired increase in the copy number of the gene. Plasmids suitable for the genus Corynebacterium are described, for example, in Tauch et al. (Journal of Biotechnology 104 (1-3), 27-40, (2003)), and in Stansen et al. (Applied and Environmental Microbiology 71, 5920-5928 (2005)).

The copy number may furthermore be increased by at least one (1) copy by introducing further copies into the chromosome of the microorganism. Methods suitable for the genus Corynebacterium are described, for example, in the patents WO 03/014330, WO 03/040373 and WO 04/069996.

Gene expression may furthermore be increased by positioning a plurality of promoters upstream of the target gene or functionally linking them to the gene to be expressed and achieving increased expression in this way. Examples of this are described in the patent WO 2006/069711.

Transcription of a gene is controlled, where appropriate, by proteins which suppress (repressor proteins) or promote (activator proteins) transcription. Accordingly, overexpression can likewise be achieved by increasing the expression of activator proteins or reducing or switching off the expression of repressor proteins or else eliminating the binding sites of the repressor proteins. The rate of elongation is influenced by the codon usage, it being possible to enhance translation by utilizing codons for transfer RNAs (tRNAs) which are frequent in the starting strain. Moreover, replacing a start codon with the ATG codon most frequent in many microorganisms (77% in E. coli) may considerably improve translation, since, at the RNA level, the AUG codon is two to three times more effective than the codons GUG and UUG, for example (Khudyakov et al., FEBS Letters 232(2):369-71(1988); Reddy et al., Proceedings of the National Academy of Sciences of the USA 82(17):5656-60 (1985)). It is also possible to optimize the sequences surrounding the start codon because synergistic effects between the start codon and the flanking regions have been described (Stenström et al., Gene 273(2):259-65 (2001); Hui et al., EMBO Journal 3(3):623-9 (1984)).

Instructions for handling DNA, digestion and ligation of DNA, transformation and selection of transformants can be found inter alia in the known manual by Sambrook et al. “Molecular Cloning: A Laboratory Manual, Second Edition (Cold Spring Harbor Laboratory Press, 1989).

The disclosure also relates to vectors comprising the polynucleotides according to the invention.

Kirchner and Tauch (Journal of Biotechnology 104:287-299 (2003)) describe a selection of vectors to be used in C. glutamicum.

Homologous recombination using the vectors according to the invention allows DNA segments on the chromosome to be replaced with polynucleotides according to the invention which are transported into the cell by the vector. For efficient recombination between the circular DNA molecule of the vector and the target DNA on the chromosome, the DNA region to be replaced with the polynucleotide according to the invention is provided at the ends with nucleotide sequences homologous to the target site which determine the site of integration of the vector and of replacement of the DNA.

Thus the promoter polynucleotide according to the invention may: 1) be replaced with the native promoter at the native gene locus of the target gene in the chromosome; or 2) be integrated with the target gene at the native gene locus of the latter or at another gene locus.

“Replacement of the native promoter at the native gene locus of the target gene” means the fact that the naturally occurring promoter of the gene which usually is naturally present by way of a single copy at its gene locus in the corresponding wild type or corresponding starting organism in the form of its nucleotide sequence is replaced. “Another gene locus” means a gene locus whose nucleotide sequence is different from the sequence of the target gene. Said other gene locus or the nucleotide sequence at said other gene locus is preferably located within the chromosome and normally is not essential for growth and for production of the desired chemical compounds. It is furthermore possible to use intergenic regions within the chromosome, i.e. nucleotide sequences without coding function.

Expression or overexpression is preferably carried out in microorganisms of the genus Corynebacterium. Within the genus Corynebacterium, preference is given to strains based on the following species: C. efficiens, with the deposited type strain being DSM44549, C. glutamicum, with the deposited type strain being ATCC13032, and C. ammoniagenes, with the deposited type strain being ATCC6871. Very particular preference is given to the species C. glutamicum.

Suitable strains of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

The term “Micrococcus glutamicus” has also been in use for C. glutamicum. Some representatives of the species C. efficiens have also been referred to as C. thermoaminogenes in the prior art, such as the strain FERM BP-1539, for example.

The microorganisms or strains (starting strains) employed for the expression or overexpression measures according to the invention preferably already possess the ability to secrete a desired fine chemical into the surrounding nutrient medium and accumulate there. The expression “to produce” is also used for this herein below. More specifically, the strains employed for the overexpression measures possess the ability to accumulate the desired fine chemical in concentrations of at least 0.10 g/L, at least 0.25 g/L, at least 0.5 g/L, at least 1.0 g/L, at least 1.5 g/L, at least 2.0 g/L, at least 4.0 g/L, or at least 10.0 g/L in no more than 120 hours, no more than 96 hours, no more than 48 hours, no more than 36 hours, no more than 24 hours, or no more than 12 hours in the cell or in the nutrient medium. The starting strains are preferably strains prepared by mutagenesis and selection, by recombinant DNA technologies or by a combination of both methods.

A person skilled in the art understands that a microorganism suitable for the measures of the invention may also be obtained by firstly employing the promoter according to the invention, e.g., SEQ ID NO:1, SEQ ID NO:2, SEQ ID NO:3, SEQ ID NO:4, SEQ ID NO:5, SEQ ID NO:6, SEQ ID NO:7, or SEQ ID NO:8 for overexpression or underexpression of the target genes in a wild strain such as, for example, the C. glutamicum type strain ATCC 13032 or the strain ATCC 14067, and then, by means of further genetic measures described in the prior art, causing the microorganism to produce the desired fine chemical(s).

The term “biomolecules” means with regard to the measures of the invention amino acids, organic acids, vitamins, nucleosides and nucleotides. Particular preference is given to proteinogenic amino acids, non-proteinogenic amino acids, macromolecules, and organic acids.

“Proteinogenic amino acids” mean the amino acids which occur in natural proteins, i.e. in proteins of microorganisms, plants, animals and humans. They serve as structural units for proteins in which they are linked to one another via peptide bonds.

Where L-amino acids or amino acids are mentioned hereinbelow, they are to be understood as meaning one or more amino acids, including their salts, selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-lysine is especially preferred. L-Amino acids, in particular lysine, are used in human medicine and in the pharmaceuticals industry, in the foodstuffs industry and very particularly in animal nutrition. There is therefore a general interest in providing new improved processes for the preparation of amino acids, in particular L-lysine.

The terms protein and polypeptide are interchangeable.

The present disclosure provides a microorganism which produces a fine chemical, said microorganism having increased expression of one or more genes in comparison to the particular starting strain by using a promoter of a promoter ladder, such as a promoter selected from SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

### Fermentative Preparation

The present disclosure furthermore provides a process for fermentative preparation of a fine chemical, comprising the steps of:

a) culturing the above-described microorganism according to the present disclosure in a suitable medium, resulting in a fermentation broth; and

b) concentrating the fine chemical in the fermentation broth of a) and/or in the cells of the microorganism.

Preference is given here to obtaining from the fine chemical-containing fermentation broth the fine chemical or a liquid or solid fine chemical-containing product. The microorganisms produced may be cultured continuously—as described, for example, in WO 05/021772—or discontinuously in a batch process (batch cultivation) or in a fed-batch or repeated fed-batch process for the purpose of producing the desired organic-chemical compound. A summary of a general nature about known cultivation methods is available in the textbook by Chmiel (Bioprozeßtechnik. 1: Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)).

The culture medium or fermentation medium to be used must in a suitable manner satisfy the demands of the respective strains. Descriptions of culture media for various microorganisms are present in the “Manual of Methods for General Bacteriology” of the American Society for Bacteriology (Washington D.C., USA, 1981). The terms culture medium and fermentation medium are interchangeable.

It is possible to use, as carbon source, sugars and carbohydrates such as, for example, glucose, sucrose, lactose, fructose, maltose, molasses, sucrose-containing solutions from sugar beet or sugar cane processing, starch, starch hydrolysate, and cellulose; oils and fats such as, for example, soybean oil, sunflower oil, groundnut oil and coconut fat; fatty acids such as, for example, palmitic acid, stearic acid, and linoleic acid; alcohols such as, for example, glycerol, methanol, and ethanol; and organic acids such as, for example, acetic acid or lactic acid.

It is possible to use, as nitrogen source, organic nitrogen-containing compounds such as peptones, yeast extract, meat extract, malt extract, corn steep liquor, soybean flour, and urea; or inorganic compounds such as ammonium sulfate, ammonium chloride, ammonium phosphate, ammonium carbonate, and ammonium nitrate. The nitrogen sources can be used individually or as a mixture.

It is possible to use, as phosphorus source, phosphoric acid, potassium dihydrogen phosphate or dipotassium hydrogen phosphate or the corresponding sodium-containing salts.

The culture medium may additionally comprise salts, for example in the form of chlorides or sulfates of metals such as, for example, sodium, potassium, magnesium, calcium and iron, such as, for example, magnesium sulfate or iron sulfate, which are necessary for growth. Finally, essential growth factors such as amino acids, for example homoserine and vitamins, for example thiamine, biotin or pantothenic acid, may be employed in addition to the abovementioned substances.

Said starting materials may be added to the culture in the form of a single batch or be fed in during the cultivation in a suitable manner.

The pH of the culture can be controlled by employing basic compounds such as sodium hydroxide, potassium hydroxide, ammonia, or aqueous ammonia; or acidic compounds such as phosphoric acid or sulfuric acid in a suitable manner. The pH is generally adjusted to a value of from 6.0 to 8.5, preferably 6.5 to 8. To control foaming, it is possible to employ antifoams such as, for example, fatty acid polyglycol esters. To maintain the stability of plasmids, it is possible to add to the medium suitable selective substances such as, for example, antibiotics. The fermentation is preferably carried out under aerobic conditions. In order to maintain these conditions, oxygen or oxygen-containing gas mixtures such as, for example, air are introduced into the culture. It is likewise possible to use liquids enriched with hydrogen peroxide. The fermentation is carried out, where appropriate, at elevated pressure, for example at an elevated pressure of from 0.03 to 0.2 MPa. The temperature of the culture is normally from 20° C. to 45° C. and preferably from 25° C. to 40° C., particularly preferably from 30° C. to 37° C. In batch or fed-batch processes, the cultivation is preferably continued until an amount of the desired organic-chemical compound sufficient for being recovered has formed. This aim is normally achieved within 10 hours to 160 hours. In continuous processes, longer cultivation times are possible. The activity of the microorganisms results in a concentration (accumulation) of the organic-chemical compound in the fermentation medium and/or in the cells of said microorganisms.

Examples of suitable fermentation media can be found inter alia in the U.S. Pat. Nos. 5,770,409, 5,990,350, 5,275,940, WO 2007/012078, U.S. Pat. No. 5,827,698, WO 2009/043803, U.S. Pat. Nos. 5,756,345 and 7,138,266.

Analysis of L-amino acids to determine the concentration at one or more time(s) during the fermentation can take place by separating the L-amino acids by means of ion exchange chromatography, preferably cation exchange chromatography, with subsequent post-column derivatization using ninhydrin, as described in Spackman et al. (Analytical Chemistry 30:1190-1206 (1958)). It is also possible to employ ortho-phthaldialdehyde rather than ninhydrin for post-column derivatization. An overview article on ion exchange chromatography can be found in Pickering (LC-GC Magazine of Chromatographic Science) 7(6), 484-487 (1989)).

It is likewise possible to carry out a pre-column derivatization, for example using ortho-phthaldialdehyde or phenyl isothiocyanate, and to fractionate the resulting amino acid derivatives by reversed-phase (RP) chromatography, preferably in the form of high-performance liquid chromatography (HPLC). A method of this type is described, for example, in Lindroth et al. (Analytical Chemistry 51:1167-1174 (1979)).

Detection is carried out photometrically (absorption, fluorescence).

A review regarding amino acid analysis can be found inter alia in the textbook “Bioanalytik” from Lottspeich and Zorbas (Spektrum Akademischer Verlag, Heidelberg, Germany 1998).

Determination of the concentration of α-ketoacids at one or more time point(s) in the course of the fermentation may be carried out by separating the ketoacids and other secreted products by means of ion exchange chromatography, preferably cation exchange chromatography, on a sulfonated styrene-divinylbenzene polymer in the H+ form, for example by means of 0.025 M sulfuric acid with subsequent UV detection at 215 nm (alternatively also at 230 or 275 nm). Preferably, a REZEK RFQ-Fast Fruit H+ column (Phenomenex) may be employed, but other suppliers for the separating phase (e.g. Aminex from BioRad) are feasible. Similar separations are described in application examples by the suppliers.

The performance of the processes or fermentation processes containing the promoter variants according to the invention, in terms of one or more of the parameters selected from the group of concentration (compound formed per unit volume), yield (compound formed per unit carbon source consumed), formation (compound formed per unit volume and time) and specific formation (compound formed per unit dry cell matter or dry biomass and time or compound formed per unit cellular protein and time) or else other process parameters and combinations thereof, is increased by at least 0.5%, at least 1%, at least 1.5%, at least 2%, at least 3%, at least 4%, at least 5%, at least 10%, at least 20%, at least 30%, at least 40%, at least 50%, at least 60%, at least 70%, at least 80%, at least 90%, or at least 100% based on processes or fermentation processes using microorganisms not containing the promoter variants according to the invention. This is considered to be very worthwhile in terms of a large-scale industrial process.

The fermentation measures result in a fermentation broth which contains the desired fine chemical, preferably amino acids, organic acids, vitamins, nucleosides or nucleotides.

A product containing the fine chemical is then provided or produced or recovered in liquid or solid form.

A fermentation broth means a fermentation medium or nutrient medium in which a microorganism has been cultivated for a certain time and at a certain temperature. The fermentation medium or the media employed during fermentation comprise(s) all the substances or components which ensure production of the desired compound and typically propagation and viability.

When the fermentation is complete, the resulting fermentation broth accordingly comprises:

a) the biomass (cell mass) of the microorganism, said biomass having been produced due to propagation of the cells of said microorganism;

b) the desired fine chemical formed during the fermentation;

c) the organic byproducts possibly formed during the fermentation; and

d) the constituents of the fermentation medium employed or of the starting materials, such as, for example, vitamins such as biotin or salts such as magnesium sulfate, which have not been consumed in the fermentation.

The organic byproducts include substances which are produced by the microorganisms employed in the fermentation in addition to the particular desired compound and are optionally secreted.

The fermentation broth is removed from the culture vessel or fermentation tank, collected where appropriate, and used for providing a product containing the fine chemical in liquid or solid form. The expression “recovering the fine chemical-containing product” is also used for this. In the simplest case, the fine chemical-containing fermentation broth itself, which has been removed from the fermentation tank, constitutes the recovered product.

One or more of the measures selected from the group consisting of

a) partial (>0% to <80%) to complete (100%) or virtually complete (≥80%, ≥90%, ≥95%, ≥96%, ≥97%, ≥98%, or ≥99%) removal of the water;

b) partial (>0% to <80%) to complete (100%) or virtually complete (≥80%, ≥90%, ≥95%, ≥96%, ≥97%, ≥98%, or ≥99%) removal of the biomass, the latter being optionally inactivated before removal;

c) partial (>0% to <80%) to complete (100%) or virtually complete (≥80%, ≥90%, ≥95%, ≥96%, ≥97%, ≥98%, ≥99%, ≥99.3%, or ≥99.7%) removal of the organic byproducts formed during fermentation; and

d) partial (>0%) to complete (100%) or virtually complete (≥80%, ≥90%, ≥95%, ≥96%, ≥97%, ≥98%, ≥99%, ≥99.3%, or ≥99.7%) removal of the constituents of the fermentation medium employed or of the starting materials, which have not been consumed in the fermentation, from the fermentation broth achieves concentration or purification of the desired organic-chemical compound. Products having a desired content of said compound are isolated in this way.

The partial (>0% to <80%) to complete (100%) or virtually complete (≥80% to <100%) removal of the water (measure a)) is also referred to as drying.

In one variant of the process, complete or virtually complete removal of the water, of the biomass, of the organic byproducts and of the unconsumed constituents of the fermentation medium employed results in pure (≥80% by weight, ≥90% by weight) or high-purity (≥95% by weight, ≥97% by weight, or ≥99% by weight) product forms of the desired organic-chemical compound. An abundance of technical instructions for measures a), b), c) and d) are available in the prior art.

Depending on requirements, the biomass can be removed wholly or partly from the fermentation broth by separation methods such as, for example, centrifugation, filtration, decantation or a combination thereof, or be left completely therein. Where appropriate, the biomass or the biomass-containing fermentation broth is inactivated during a suitable process step, for example by thermal treatment (heating) or by addition of acid.

In one procedure, the biomass is completely or virtually completely removed so that no (0%) or at most 30%, at most 20%, at most 10%, at most 5%, at most 1% or at most 0.1% biomass remains in the prepared product. In a further procedure, the biomass is not removed, or is removed only in small proportions, so that all (100%) or more than 70%, 80%, 90%, 95%, 99% or 99.9% biomass remains in the product prepared. In one process according to the invention, accordingly, the biomass is removed in proportions of from ≥0% to ≤100%.

Finally, the fermentation broth obtained after the fermentation can be adjusted, before or after the complete or partial removal of the biomass, to an acidic pH with an inorganic acid such as, for example, hydrochloric acid, sulfuric acid, or phosphoric acid; or organic acid such as, for example, propionic acid, so as to improve the handling properties of the final product (GB 1,439,728 or EP 1 331220). It is likewise possible to acidify the fermentation broth with the complete content of biomass. Finally, the broth can also be stabilized by adding sodium bisulfite (NaHCO₃, GB 1,439,728) or another salt, for example ammonium, alkali metal, or alkaline earth metal salt of sulfurous acid.

During the removal of the biomass, any organic or inorganic solids present in the fermentation broth are partially or completely removed. The organic byproducts dissolved in the fermentation broth, and the dissolved unconsumed constituents of the fermentation medium (starting materials), remain at least partly (>0%), preferably to an extent of at least 25%, particularly preferably to an extent of at least 50% and very particularly preferably to an extent of at least 75% in the product. Where appropriate, they also remain completely (100%) or virtually completely, meaning >95% or >98% or >99%, in the product. If a product in this sense comprises at least part of the constituents of the fermentation broth, this is also described by the term “product based on fermentation broth”.

Subsequently, water is removed from the broth, or said broth is thickened or concentrated, by known methods such as, for example, using a rotary evaporator, thin-film evaporator, falling-film evaporator, by reverse osmosis or by nanofiltration. This concentrated fermentation broth can then be worked up to free-flowing products, in particular to a fine powder or preferably coarse granules, by methods of freeze drying, spray drying, spray granulation or by other processes such as in the circulating fluidized bed, as described for example according to PCT/EP2004/006655. A desired product is isolated where appropriate from the resulting granules by screening or dust removal. It is likewise possible to dry the fermentation broth directly, i.e. without previous concentration by spray drying or spray granulation.

“Free-flowing” means powders which, from a series of glass orifice vessels with orifices of different sizes, flow unimpeded at least out of the vessel with a 5 mm orifice (Klein: Seifen, Öle, Fette, Wachse 94, 12 (1968)).

“Fine” means a powder predominantly (>50%) having a particle size of diameter from 20 to 200 μm.

“Coarse” means a product predominantly (>50%) of a particle size of diameter from 200 to 2000 μm.

The particle size determination can be carried out by methods of laser diffraction spectrometry. Corresponding methods are described in the textbook “TeilchengroBenmessung in der Laborpraxis” by R. H. Müller and R. Schuhmann, Wissenschaftliche Verlagsgesellschaft Stuttgart (1996) or in the text book “Introduction to Particle Technology” by M. Rhodes, published by Wiley &Sons (1998).

The free-flowing, fine powder can in turn be converted by suitable compaction or granulation processes into a coarse, very free-flowing, storable and substantially dust-free product.

The term “dust-free” means that the product comprises only small proportions (<5%) of particle sizes below 100 μm in diameter.

“Storable” in the sense of this invention means a product which can be stored for at least one (1) year or longer, preferably at least 1.5 years or longer, particularly preferably two (2) years or longer, in a dry and cool environment without any substantial loss of the respective organic-chemical compound occurring. “Substantial loss” means a loss of >5%.

It is advantageous to employ during the granulation or compaction the usual organic or inorganic auxiliaries or carriers such as starch, gelatin, cellulose derivatives or similar substances, as normally used in the processing of food products or feeds as binders, gelling agents or thickeners, or further substances such as, for example, silicas, silicates (EP0743016A) and stearates.

It is further advantageous to treat the surface of the resulting granules with oils or fats as described in WO04/054381. Oils which can be used are mineral oils, vegetable oils or mixtures of vegetable oils. Examples of such oils are soybean oil, olive oil, soybean oil/lecithin mixtures. In the same way, silicone oils, polyethylene glycols or hydroxyethylcellulose are also suitable. Treatment of the surfaces of the granules with said oils achieves an increased abrasion resistance of the product and a reduction in the dust content. The oil content in the product is 0.02 to 2.0% by weight, preferably 0.02 to 1.0% by weight, and very particularly preferably 0.2 to 1.0% by weight, based on the total amount of the feed additive.

Preferred products have a proportion of ≥97% by weight with a particle size of from 100 to 1800 μm or a proportion of ≥95% by weight with a particle size of diameter 300 to 1800 μm. The proportion of dust, i.e. particles with a particle size<100 μm, is preferably >0 to 1% by weight, particularly preferably not exceeding 0.5% by weight.

However, alternatively, the product may also be absorbed on an organic or inorganic carrier known and customary in the processing of feeds, such as, for example, silicas, silicates, meals, brans, flours, starches, sugars or others, and/or be mixed and stabilized with customary thickeners or binders. Examples of use and processes therefor are described in the literature (Die Mühle+Mischfuttertechnik 132 (1995) 49, page 817).

The following examples are provided for purposes of illustration, not limitation.

### EXAMPLES

### Example 1: Application of Candidate Promoters to the L-Lysine Biosynthetic Pathway

The promoters of the present disclosure are useful for improved processes for the production of biomolecules in host cells. An example of the application and use of the promotor of the present disclosure is directed to the production of the amino acid L-lysine.

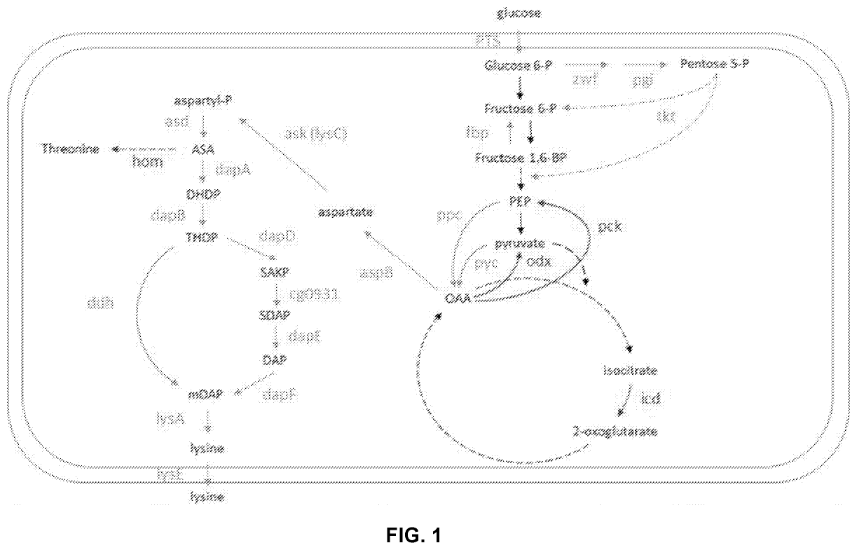

FIG. 1 presents the biosynthetic pathway for the production of L-lysine and includes the genes pck, odx, icd, and hom (e.g., the homoserine/threonine synthase pathway), that divert intermediates from the pathway leading to reductions in overall L-lysine yield. The symbols, gene names, Enzyme Commission number (EC number), and map position in C. glutamicum strain ATCC 13032 are provided in Table 3.

Recombinant vectors comprising a promoter of SEQ ID NOs: 1 to 8 functionally linked to a target gene as provided in Table 3 are cloned into Corynebacterium cloning vectors using yeast homologous recombination cloning techniques to assemble a vector in which each promoter was flanked by direct repeat regions to provide for homologous recombination in Corynebacterium glutamicum at the target gene locus. Upon recombination, the endogenous promoter is replaced by the promoter of SEQ ID NOs: 1 to 8 functionally linked to the respective target gene in the endogenous C. glutamicum locus. A variety of targeting vectors comprising the promoter and functionally linked target gene included a range of homology direct repeat arm lengths ranging from 0.5 Kb, 1 Kb, 2 Kb, and 5 Kb. Each DNA insert was produced by PCR amplification of homologous regions using commercially sourced oligos and the host strain genomic DNA described above as template. The promoter to be introduced into the genome was encoded in the oligo tails. PCR fragments were assembled into the vector backbone using homologous recombination in yeast.

Vectors are initially transformed into E. coli using standard heat shock transformation techniques and correctly assembled clones are identified and validated. Transformed E. coli bacteria are tested for assembly success. Four colonies from each E. coli transformation plate are cultured and tested for correct assembly via PCR. Vectors are amplified in the E. coli hosts to provide vector DNA for Corynebacterium transformation.

Validated clones are transformed into Corynebacterium glutamicum host cells via electroporation. For each transformation, the number of Colony Forming Units (CFUs) per μg of DNA is determined as a function of the insert size. Corynebacterium genome integration is analyzed as a function of homology arm length. Shorter arms had a lower efficiency.

Cultures of Corynebacterium identified as having successful integrations of the insert cassette are cultured on media containing 5% sucrose to counter select for loop outs of the sacb selection gene. Sucrose resistance frequency for various homology direct repeat arms do not vary significantly with arm length. These results suggest that loopout efficiencies remain steady across homology arm lengths of 0.5 kb to 5 kb.

In order to further validate loop out events, colonies exhibiting sucrose resistance are cultured and analyzed via sequencing. The results for the sequencing of the insert genomic regions are summarized below in Table 6.

TABLE 6
Loop-out Validation Frequency

| Outcome | Frequency (sampling error 95% confidence) | |
|---|---|---|
| Successful | 13% | (9%/20%) |
| Loop out | | |
| Loop Still | 42% | (34%/50%) |
| present | | |
| Mixed read | 44% | (36%/52%) |

Sequencing results show a 10-20% efficiency in loop outs. Not to be limited by any particular theory, loop-out may be dependent on insert sequence. Even if correct, picking 10-20 sucrose-resistant colonies leads to high success rates.

Upon integration, the recombinant vectors replace the endogenous promoter sequences with a promoter selected from the group consisting of Pcg1860 (SEQ ID NO:2), Pcg0007 (SEQ ID NO:3), Pcg0755 (SEQ ID NO:4), Pcg0007_lib_265 (SEQ ID NO:5), Pcg3381 (SEQ ID NO:6), Pcg007_lib_119 (SEQ ID NO:7), and Pcg3121 (SEQ ID NO:8). The resulting recombinant strains is provided in the following list:

Pcg1860-asd; Pcg0755-asd; Pcg0007_119-asd; Pcg3121-asd; Pcg0007_265-asd; Pcg3381-asd; Pcg1860-ask; Pcg0755-ask; Pcg3121-ask; Pcg0007_119-ask; Pcg0007_265-ask; Pcg3381-ask; Pcg3381-aspB; Pcg0007_119-aspB; Pcg0007_119-cg0931; Pcg1860-cg0931; Pcg0007_265-cg0931; Pcg0007_39-cg0931; Pcg0755-cg0931; Pcg0007-cg0931; Pcg0007-dapA; Pcg3381-dapA; Pcg0007_265-dapA; Pcg0007_119-dapA; Pcg0007_265-dapB; Pcg0755-dapB; Pcg0007-dapB; Pcg3381-dapB; Pcg1860-dapB Pcg3121-dapB; Pcg0007_119-dapB; Pcg0007_265-dapD; Pcg0007_119-dapD; Pcg3381-dapD; Pcg0007_39-dapD; Pcg3121-dapD; Pcg0007-dapD; Pcg1860-dapD; Pcg0755-dapD; Pcg3381-dapE; Pcg3121-dapE; Pcg0755-dapE; Pcg0007_119-dapE; Pcg1860-dapE; Pcg0007_39-dapE; Pcg0007_265-dapF; Pcg3381-dapF; Pcg0007_119-dapF; Pcg0007-dapF; Pcg1860-dapF; Pcg0007_39-dapF; Pcg3381-ddh; Pcg3121-ddh; Pcg0007_119-ddh; Pcg0007_39-ddh; Pcg1860-ddh; Pcg0007_265-ddh; Pcg0755-ddh; Pcg0007-ddh; Pcg3381-fbp; Pcg0007_119-fbp; Pcg1860-fbp; Pcg0007-fbp; Pcg3121-fbp; Pcg0755-fbp; Pcg0755-hom; Pcg3381-hom; Pcg1860-hom; Pcg3121-hom; Pcg0007_119-icd; Pcg3121-icd; Pcg3381-icd; Pcg1860-icd; Pcg0007_39-icd; Pcg0007-icd; Pcg0007_265-icd; Pcg0007-lysA; Pcg0007_39-lysA; Pcg3121-lysA; Pcg0007_265-lysA; Pcg0007_119-lysA; Pcg3381-lysA; Pcg0007_39-lysE; Pcg0007-lysE; Pcg0007_265-lysE; Pcg3121-lysE; Pcg3381-lysE; Pcg0007_119-lysE; Pcg3381-odx; Pcg0007_265-odx; Pcg0755-odx; Pcg0007-odx; Pcg1860-odx; Pcg0007_39-odx; Pcg0007_119-odx; Pcg3121-odx; Pcg3121-pck; Pcg3381-pck; Pcg0007_119-pck; Pcg0007_265-pck; Pcg0755-pck; Pcg0007_39-pck; Pcg0007-pck; Pcg1860-pck; Pcg3121-pgi; Pcg0007_119-pgi; Pcg3381-pgi; Pcg0007_265-pgi; Pcg1860-pgi; Pcg0007-pgi; Pcg0007_39-ppc; Pcg0007_265-ppc; Pcg0755-ppc; Pcg3381-ppc; Pcg0007_119-ppc; Pcg1860-ppc; Pcg3121-ppc; Pcg0755-ptsG; Pcg1860-ptsG; Pcg0007_39-ptsG; Pcg3381-ptsG; Pcg0007_119-ptsG; Pcg3121-ptsG; Pcg1860-pyc; Pcg0755-pyc; Pcg0007_39-pyc; Pcg0007_265-pyc; Pcg0007-pyc; Pcg3381-pyc; Pcg0007_119-pyc; Pcg3121-pyc; Pcg3121-tkt; Pcg0007_119-tkt; Pcg0755-tkt; Pcg0007-tkt; Pcg3381-tkt; Pcg0007_265-tkt; Pcg0007-zwf; Pcg0755-zwf; Pcg0007_265-zwf; and Pcg1860-zwf; Pcg3121-zwf.

Multiple single colonies are picked, inoculated and grown as a small scale culture. Each newly created strain comprising a test promoter is tested for lysine yield in small scale cultures designed to assess product titer performance. Small scale cultures are conducted using media from industrial scale cultures. Product titer is optically measured at carbon exhaustion (i.e., representative of single batch yield) with a standard colorimetric assay. Briefly, a concentrated assay mixture is prepared and is added to fermentation samples such that final concentrations of reagents are 160 mM sodium phosphate buffer, 0.2 mM Amplex Red, 0.2 U/mL Horseradish Peroxidase and 0.005 U/mL of lysine oxidase. Reactions proceed to completion and optical density is measured using a Tecan M1000 plate spectrophotometer at a 560 nm wavelength.

In some cases, the yield of L-lysine is increased by over 24% (e.g., recombinant strain 7000007840) over the non-engineered strain. In other embodiments, the yield of L-lysine is decreased by nearly 90% (e.g., recombinant strain 700000773). Replacement of the promoter for the pgi and zwf results in greater than 10% improvements to L-lysine production.

Notably, the production of L-lysine is not a simple dependence on incorporating the most active promoters. Lysine yield is maximized by a relatively weak promoter (e.g., pgi having relative promoter expression of 1, 7×, or 48×, or dapB at a relative promoter strength of 7×) or maximized by intermediate expression (e.g., lysA at having a relative promoter expression of 454×). In certain cases, expression is maximal when the relative promoter strength is maximized (e.g., ppc). The location of the gene in the genetic pathway does not reliably predict the relative increase or decrease in L-lysine yield or the optimal promoter strength. For example, high level expression of cg0931 results in improved yield while higher levels of dapD result in no improvement or decreased yield.

### Example 2: Engineering the L-Lysine Biosynthetic Pathway

The yield of L-lysine is modified by swapping pairs of promoters for target genes. The constructs of Example 1 are used to prepare recombinant organsims as follows:

The combination of Pcg0007-lysA and Pcg3121-pgi provide for the highest yields of L-lysine.

TABLE 7
Paired Promoter Swapping of Target Genes in the L-lysine biosynthetic pathway

| | | | | Mean Yield | |
|---|---|---|---|---|---|
| Strain ID | Number | PRO Swap 1 | PRO Swap 2 | (A₅₆₀) | Std Dev |
| 7000008489 | 4 | Pcg0007-lysA | Pcg3121-pgi | 1.17333 | 0.020121 |
| 7000008530 | 8 | Pcg1860-pyc | Pcg0007-zwf | 1.13144 | 0.030023 |
| 7000008491 | 7 | Pcg0007-lysA | Pcg0007-zwf | 1.09836 | 0.028609 |
| 7000008504 | 8 | Pcg3121-pck | Pcg0007-zwf | 1.09832 | 0.021939 |
| 7000008517 | 8 | Pcg0007_39-ppc | Pcg0007-zwf | 1.09502 | 0.030777 |
| 7000008502 | 4 | Pcg3121-pck | Pcg3121-pgi | 1.09366 | 0.075854 |
| 7000008478 | 4 | Pcg3381-ddh | Pcg0007-zwf | 1.08893 | 0.025505 |
| 7000008465 | 4 | Pcg0007_265-dapB | Pcg0007-zwf | 1.08617 | 0.025231 |
| 7000008535 | 8 | Pcg0007-zwf | Pcg3121-pgi | 1.06261 | 0.019757 |
| 7000008476 | 6 | Pcg3381-ddh | Pcg3121-pgi | 1.04808 | 0.084307 |
| 7000008510 | 8 | Pcg3121-pgi | Pcg1860-pyc | 1.04112 | 0.021087 |
| 7000008525 | 8 | Pcg1860-pyc | Pcg0007_265-dapB | 1.0319 | 0.034045 |
| 7000008527 | 8 | Pcg1860-pyc | Pcg0007-lysA | 1.02278 | 0.043549 |
| 7000008452 | 5 | Pcg1860-asd | Pcg0007-zwf | 1.02029 | 0.051663 |
| 7000008463 | 4 | Pcg0007_265-dapB | Pcg3121-pgi | 1.00511 | 0.031604 |
| 7000008524 | 8 | Pcg1860-pyc | Pcg1860-asd | 1.00092 | 0.026355 |
| 7000008458 | 4 | Pcg3381-aspB | Pcg1860-pyc | 1.00043 | 0.020083 |
| 7000008484 | 8 | Pcg3381-fbp | Pcg1860-pyc | 0.99686 | 0.061364 |
| 7000008474 | 8 | Pcg3381-ddh | Pcg3381-fbp | 0.99628 | 0.019733 |
| 7000008522 | 8 | Pcg0755-ptsG | Pcg3121-pgi | 0.99298 | 0.066021 |
| 7000008528 | 8 | Pcg1860-pyc | Pcg3121-pck | 0.99129 | 0.021561 |
| 7000008450 | 4 | Pcg1860-asd | Pcg3121-pgi | 0.98262 | 0.003107 |
| 7000008448 | 8 | Pcg1860-asd | Pcg3381-fbp | 0.97814 | 0.022285 |
| 7000008494 | 8 | Pcg0007_39-lysE | Pcg3381-fbp | 0.97407 | 0.027018 |
| 7000008481 | 8 | Pcg3381-fbp | Pcg0007-lysA | 0.9694 | 0.029315 |
| 7000008497 | 8 | Pcg0007_39-lysE | Pcg1860-pyc | 0.9678 | 0.028569 |
| 7000008507 | 8 | Pcg3121-pgi | Pcg3381-fbp | 0.96358 | 0.035078 |
| 7000008501 | 8 | Pcg3121-pck | Pcg0007-lysA | 0.96144 | 0.018665 |
| 7000008486 | 8 | Pcg0007-lysA | Pcg0007_265-dapB | 0.94523 | 0.017578 |
| 7000008459 | 8 | Pcg0007_265-dapB | Pcg1860-asd | 0.94462 | 0.023847 |
| 7000008506 | 2 | Pcg3121-pgi | Pcg0007_265-dapD | 0.94345 | 0.014014 |
| 7000008487 | 8 | Pcg0007-lysA | Pcg3381-ddh | 0.94249 | 0.009684 |
| 7000008498 | 8 | Pcg3121-pck | Pcg1860-asd | 0.94154 | 0.016802 |
| 7000008485 | 8 | Pcg0007-lysA | Pcg1860-asd | 0.94135 | 0.013578 |
| 7000008499 | 8 | Pcg3121-pck | Pcg0007_265-dapB | 0.93805 | 0.013317 |
| 7000008472 | 8 | Pcg3381-ddh | Pcg1860-asd | 0.93716 | 0.012472 |
| 7000008511 | 8 | Pcg0007_39-ppc | Pcg1860-asd | 0.93673 | 0.015697 |
| 7000008514 | 8 | Pcg0007_39-ppc | Pcg0007-lysA | 0.93668 | 0.027204 |
| 7000008473 | 8 | Pcg3381-ddh | Pcg0007_265-dapB | 0.93582 | 0.030377 |
| 7000008461 | 7 | Pcg0007_265-dapB | Pcg3381-fbp | 0.93498 | 0.037862 |
| 7000008512 | 8 | Pcg0007_39-ppc | Pcg0007_265-dapB | 0.93033 | 0.017521 |
| 7000008456 | 8 | Pcg3381-aspB | Pcg3121-pck | 0.92544 | 0.020075 |
| 7000008460 | 8 | Pcg0007_265-dapB | Pcg0007_265-dapD | 0.91723 | 0.009508 |
| 7000008492 | 8 | Pcg0007_39-lysE | Pcg3381-aspB | 0.91165 | 0.012988 |
| 7000008493 | 8 | Pcg0007_39-lysE | Pcg0007_265-dapD | 0.90609 | 0.031968 |
| 7000008453 | 8 | Pcg3381-aspB | Pcg0007_265-dapB | 0.90338 | 0.013228 |
| 7000008447 | 8 | Pcg1860-asd | Pcg0007_265-dapD | 0.89886 | 0.028896 |
| 7000008455 | 8 | Pcg3381-aspB | Pcg0007-lysA | 0.89531 | 0.027108 |
| 7000008454 | 6 | Pcg3381-aspB | Pcg3381-ddh | 0.87816 | 0.025807 |
| 7000008523 | 8 | Pcg0755-ptsG | Pcg1860-pyc | 0.87693 | 0.030322 |
| 7000008520 | 8 | Pcg0755-ptsG | Pcg3381-fbp | 0.87656 | 0.018452 |
| 7000008533 | 4 | Pcg0007-zwf | Pcg3381-fbp | 0.84584 | 0.017012 |
| 7000008519 | 8 | Pcg0755-ptsG | Pcg0007_265-dapD | 0.84196 | 0.025747 |

### Example 3: Engineering the L-Lysine Biosynthetic Pathway with Promoters Operably Linked to Off-Pathway Genes

The yield of L-lysine is modified by including a second promoter polynucleotide sequence functionally linked to an off-pathway second heterologous target gene. The heterologous target genes are selected from ncg10009, ncg10019, ncg10054, ncg10082, ncg10142, ncg10223, ncg10241, ncg10242, ncg10304, ncg10306, ncg10356, ncg10398, ncg10408, ncg10424, ncg10425, ncg10427, ncg10439, ncg10458, ncg10471, ncg10531, ncg10546, ncg10564, ncg10573, ncg10578, ncg10581, ncg10598, ncg10600, ncg10601, ncg10641, ncg10663, ncg10668, ncg10737, ncg10767, ncg10813, ncg10823, ncg10827, ncg10853, ncg10874, ncg10877, ncg10905, ncg10916, ncg10966, ncg11065, ncg11124, ncg11137, ncg11152, ncg11187, ncg11196, ncg11202, ncg11203, ncg11208, ncg11261, ncg11262, ncg11267, ncg11301, ncg11320, ncg11322, ncg11364, ncg11366, ncg11371, ncg11372, ncg11457, ncg11484, ncg11500, ncg11503, ncg11508, ncg11511, ncg11545, ncg11550, ncg11583, ncg11607, ncg11855, ncg11858, ncg11880, ncg11886, ncg11900, ncg11905, ncg11911, ncg11928, ncg11948, ncg11961, ncg12001, ncg12002, ncg12019, ncg12048, ncg12077, ncg12104, ncg12147, ncg12153, ncg12190, ncg12204, ncg12210, ncg12211, ncg12247, ncg12250, ncg12274, ncg12286, ncg12287, ncg12298, ncg12327, ncg12399, ncg12425, ncg12440, ncg12441, ncg12446, ncg12449, ncg12472, ncg12473, ncg12481, ncg12491, ncg12505, ncg12527, ncg12535, ncg12538, ncg12559, ncg12567, ncg12569, ncg12576, ncg12587, ncg12614, ncg12669, ncg12684, ncg12699, ncg12702, ncg12755, ncg12789, ncg12790, ncg12802, ncg12827, ncg12886, ncg12898, ncg12901, ncg12905, ncg12921, ncg12929, ncg12930, ncg12931, ncg12982, and ncg12984.

Constructs containing a promoter identified herein linked to sequences homologous to a portion of the heterologous off-pathway genes identified above are used to prepare recombinant host cell organisms as provided in Tables 8 and 9. Upon integration, the recombinant vectors replace the endogenous promoter sequences with a promoter selected from the group consisting of Pcg1860 (SEQ ID NO:2), Pcg0007 (SEQ ID NO:3), Pcg0755 (SEQ ID NO:4), Pcg0007_lib_265 (SEQ ID NO:5), Pcg3381 (SEQ ID NO:6), Pcg007_lib_119 (SEQ ID NO:7), and Pcg3121 (SEQ ID NO:8). A list of the resulting recombinant strains is provided below in Table 8.

Multiple single colonies (N in Table 8) are picked, inoculated and grown as a small scale culture. Each newly created strain comprising a test promoter is tested for lysine yield in small scale cultures designed to assess product titer performance. Small scale cultures are conducted using media from industrial scale cultures. Product titer is optically measured at carbon exhaustion (i.e., representative of single batch yield) with a standard colorimetric assay. Briefly, a concentrated assay mixture is prepared and is added to fermentation samples such that final concentrations of reagents are 160 mM sodium phosphate buffer, 0.2 mM Amplex Red, 0.2 U/mL Horseradish Peroxidase and 0.005 U/mL of lysine oxidase. Reactions proceed to completion and optical density is measured using a Tecan M1000 plate spectrophotometer at a 560 nm wavelength.

As shown in Table 8, the yield of L-lysine is increased by over 14% (e.g., recombinant strain 7000152451) over the parent strain that does not contain a heterologous promoter functionally linked to an off-pathway target gene. Among those promoter replacements applied in at least three different strains in Table 9, the best performing modifications overall are pcg0007_39-cg0725 (average of 6.5% yield change in six strains), pcg0007_39-ncg11262 (average of 6.3% yield change in nine strains), and pcg0007_39-cg2766 (average of 5.1% yield change in 23 strains).

Notably, the production of L-lysine is not a simple dependence on incorporating the most active promoters. The pcg3121-mutm22522 modification involves a weak promoter but improved yield by an average of 5% in four strains.

TABLE 8
Recombinant strains of C. glutamicum having modified expression of non-L-lysine
Biosynthetic Genes and yield change from base of at least 3%, where the promoter-
target modification has been applied in at least five different strain backgrounds

| | | | | | % Yield |
|---|---|---|---|---|---|
| | | | Mean | | Change |
| Strain | promoter-target | N | (A₅₆₀) | Std Error | From Base |
| 7000011650 | pcg0007_39-dnak | 16 | 0.939907 | 0.005637 | 14.40616 |
| 7000011837 | pcg0007_39-cg0074 | 8 | 0.924085 | 0.005601 | 12.48029 |
| 7000012092 | pcg3121-cg0074 | 8 | 0.927409 | 0.006967 | 12.88495 |
| 7000051494 | pcg1860-rhle_609 | 14 | 1.060682 | 0.005131 | 8.870004 |
| 7000051495 | pcg3121-cg1144 | 16 | 1.055172 | 0.0054 | 8.30446 |
| 7000071062 | pcg1860-rhle_609 | 18 | 0.744484 | 0.007673 | 3.518385 |
| 7000101786 | pcg0007_39-cg2899_2194 | 20 | 1.059718 | 0.00367 | 3.920982 |
| 7000101932 | pcg0007_39-cg1486 | 6 | 1.057967 | 0.008648 | 3.749293 |
| 7000101946 | pcg0007_39-cg2766 | 448 | 1.075533 | 0.003077 | 5.471888 |
| 7000102382 | pcg0007_39-cmk | 8 | 1.056225 | 0.007802 | 3.57847 |
| 7000132573 | pcg0007_39-rpob_383 | 12 | 1.055606 | 0.008687 | 3.517745 |
| 7000132579 | pcg0007_39-ddl | 4 | 1.099107 | 0.012557 | 4.686924 |
| 7000132585 | pcg0007_39-cg0027 | 5 | 1.078981 | 0.007576 | 5.008844 |
| 7000132587 | pcg0007_39-ddl | 5 | 1.061015 | 0.009978 | 3.260317 |
| 7000132589 | pcg0007_39-rpob_383 | 5 | 1.082512 | 0.007857 | 5.352516 |
| 7000132596 | pcg0007_39-rpob_383 | 5 | 1.107812 | 0.007265 | 6.123558 |
| 7000134570 | pcg0007_39-cg0027 | 12 | 1.071955 | 0.010682 | 9.944488 |
| 7000138780 | pcg1860-cg1144 | 16 | 1.068026 | 0.016669 | 3.061599 |
| 7000139655 | pcg0007_39-cg0725 | 28 | 1.06408 | 0.005703 | 5.890614 |
| 7000144050 | pcg0007_39-cg0027 | 8 | 1.113507 | 0.009172 | 5.81486 |
| 7000144052 | pcg0007_39-cg1527 | 6 | 1.122574 | 0.009961 | 6.676453 |
| 7000144055 | pcg0007_39-ddl | 8 | 1.114668 | 0.003544 | 5.925188 |
| 7000144056 | pcg0007_39-rpob_383 | 8 | 1.122532 | 0.004265 | 6.672479 |
| 7000148399 | pcg0007_39-cg0725 | 39 | 1.069788 | 0.007402 | 3.231703 |
| 7000148414 | pcg0007_39-cg0725 | 20 | 1.06931 | 0.008863 | 6.411116 |
| 7000148433 | pcg0007_39-ddl | 4 | 0.999022 | 0.013036 | 3.363822 |
| 7000148440 | pcg0007_39-cg0725 | 17 | 1.080813 | 0.009706 | 11.82635 |
| 7000148442 | pcg0007_39-cg2766 | 15 | 1.09271 | 0.011011 | 13.0573 |
| 7000148453 | pcg0007_39-cg0725 | 19 | 0.967318 | 0.008709 | 3.173838 |
| 7000148476 | pcg0007_39-hspr | 2 | 0.95376 | 0.0072 | 4.883646 |
| 7000148498 | pcg0007_39-cg3352 | 7 | 0.956069 | 0.01208 | 5.137602 |
| 7000148526 | pcg0007_39-cg2899_2194 | 4 | 0.9628 | 0.022005 | 5.877807 |
| 7000148917 | pcg0007_39-cg2766 | 17 | 0.966282 | 0.007737 | 6.260712 |
| 7000148950 | pcg0007_39-cg2766 | 11 | 1.101872 | 0.00755 | 6.318818 |
| 7000148952 | pcg0007_39-cg2965 | 11 | 1.068518 | 0.006968 | 3.100537 |
| 7000148963 | pcg0007_39-rpob_383 | 26 | 0.885703 | 0.01053 | 4.88738 |
| 7000148966 | pcg0007_39-cg2766 | 19 | 1.078181 | 0.00924 | 5.142825 |
| 7000149002 | pcg0007_39-cg2766 | 17 | 1.105547 | 0.003775 | 3.957767 |
| 7000149072 | pcg0007_39-cg2899_2194 | 7 | 0.938323 | 0.009912 | 3.186081 |
| 7000149133 | pcg0007_39-cg0074 | 4 | 0.949821 | 0.002412 | 4.45053 |
| 7000149138 | pcg3121-cg0074 | 6 | 0.93946 | 0.002513 | 3.311153 |
| 7000151562 | pcg0007_39-cg2766 | 22 | 1.069264 | 0.007822 | 4.196667 |
| 7000151586 | pcg3121-cg1144 | 8 | 0.948437 | 0.006684 | 4.298347 |
| 7000151646 | pcg0007_39-cg2766 | 37 | 1.073209 | 0.00667 | 3.660275 |
| 7000151755 | pcg0007_39-cg2766 | 6 | 1.07101 | 0.010452 | 12.61841 |
| 7000151756 | pcg0007_39-cg2899 | 2 | 1.080952 | 0.00245 | 13.66387 |
| 7000151757 | pcg0007_39-rho | 7 | 1.055369 | 0.010359 | 10.97379 |
| 7000151842 | pcg0007_39-cg2766 | 14 | 0.950916 | 0.007382 | 4.092383 |
| 7000151844 | pcg0007_39-cg2766 | 13 | 0.980145 | 0.005638 | 7.291958 |
| 7000151863 | pcg0007_39-cg0725 | 347 | 1.037644 | 0.003105 | 8.621402 |
| 7000151867 | pcg1860-cg1144 | 17 | 0.997554 | 0.016818 | 4.424723 |
| 7000151881 | pcg0007_39-cg2766 | 21 | 1.057606 | 0.003115 | 3.190432 |
| 7000151906 | pcg0007_39-cg2766 | 52 | 1.085568 | 0.008262 | 5.547884 |
| 7000152431 | pcg0007_39-urer | 27 | 1.082933 | 0.008288 | 4.599504 |
| 7000152450 | pcg0007_39-nusg | 27 | 0.990823 | 0.013909 | 5.68089 |
| 7000152451 | pcg3121-mutm2_2522 | 8 | 0.98005 | 0.00757 | 4.531918 |
| 7000152503 | pcg0007_39-ddl | 8 | 0.972555 | 0.00636 | 3.732443 |
| 7000152510 | pcg1860-cg1144 | 6 | 0.973355 | 0.005158 | 3.817762 |
| 7000152585 | pcg0007_39-cg2899 | 6 | 0.92449 | 0.028472 | 6.198442 |
| 7000152586 | pcg0007_39-cg2965 | 15 | 0.952111 | 0.014247 | 9.371358 |
| 7000152587 | pcg0007_39-ddl | 6 | 0.97185 | 0.010086 | 11.63879 |
| 7000152595 | pcg3121-mutm2_2522 | 7 | 1.000598 | 0.005055 | 14.94122 |
| 7000154599 | pcg0007_39-cg2766 | 32 | 1.047813 | 0.006632 | 5.038279 |
| 7000154607 | pcg0007_39-cg2766 | 64 | 1.094538 | 0.01056 | 4.462625 |
| 7000154623 | pcg0007_39-cg2766 | 12 | 1.084043 | 0.010304 | 5.347278 |
| 7000155554 | pcg0007_39-cg2766 | 76 | 1.061757 | 0.006753 | 3.655825 |
| 7000172142 | pcg0007_39-cg2766 | 23 | 0.98211 | 0.006367 | 5.011511 |
| 7000172150 | pcg0007_39-cg2766 | 20 | 1.100986 | 0.007079 | 5.411028 |
| 7000174400 | pcg0007_39-tyra | 42 | 1.082497 | 0.011274 | 3.702717 |
| 7000174421 | pcg0007_39-cg1486 | 34 | 1.087003 | 0.01147 | 4.134408 |
| 7000178668 | pcg0007_39-cg2899 | 18 | 1.091477 | 0.021824 | 3.721322 |
| 7000178693 | pcg0007_39-cg0027 | 18 | 1.091807 | 0.011304 | 3.519654 |
| 7000179790 | pcg0007_39-ncgl1511 | 13 | 1.077263 | 0.020883 | 3.484369 |
| 7000179967 | pcg0007_39-ncgl1262 | 55 | 1.127037 | 0.009118 | 8.2657 |
| 7000182541 | pcg0007_39-cg3419 | 11 | 1.109639 | 0.009557 | 5.221902 |
| 7000182553 | pcg0007_39-cg1486 | 10 | 1.121579 | 0.012964 | 6.354126 |
| 7000182556 | pcg0007_39-cg3210 | 8 | 1.093494 | 0.013361 | 3.690927 |
| 7000182560 | pcg0007_39-cg1486 | 9 | 1.111948 | 0.011145 | 5.440906 |
| 7000182594 | pcg0007_39-cg1486 | 8 | 1.030912 | 0.015987 | 3.199736 |
| 7000182604 | pcg0007_39-cg1486 | 8 | 1.080288 | 0.013249 | 4.919604 |
| 7000182620 | pcg0007_39-cg1486 | 71 | 1.121599 | 0.007064 | 3.116152 |
| 7000183003 | pcg0007_39-ncgl0767 | 8 | 1.1098 | 0.015313 | 5.23717 |
| 7000183123 | pcg0007_39-ncgl2481 | 7 | 1.098826 | 0.017557 | 4.1966 |
| 7000183674 | pcg0007_39-tyra | 11 | 1.065235 | 0.007951 | 3.264175 |
| 7000187919 | pcg0007_39-cg1486 | 8 | 1.055633 | 0.007257 | 3.038697 |
| 7000187929 | pcg0007_39-ncgl1511 | 52 | 1.074922 | 0.006759 | 5.748624 |
| 7000187963 | pcg0007_39-ncgl0827 | 8 | 1.093548 | 0.004974 | 4.32642 |
| 7000190043 | pcg0007_39-tyra | 12 | 1.105391 | 0.006829 | 3.253128 |
| 7000190074 | pcg0007_39-cg1486 | 10 | 1.119157 | 0.007057 | 3.146392 |
| 7000190089 | pcg0007_39-ncgl1262 | 24 | 1.132882 | 0.010445 | 7.067927 |
| 7000190098 | pcg0007_39-cg1486 | 12 | 1.096864 | 0.010931 | 3.142087 |
| 7000190123 | pcg0007_39-ncgl1262 | 144 | 1.11294 | 0.002915 | 5.948846 |
| 7000191520 | pcg0007_39-ncgl1262 | 117 | 1.099061 | 0.004345 | 5.441041 |
| 7000191588 | pcg0007_39-ncgl0767 | 12 | 1.096351 | 0.004382 | 4.369537 |
| 7000196624 | pcg0007_39-ncgl0304 | 18 | 1.090471 | 0.0102 | 3.809772 |
| 7000196641 | pcg0007_39-ncgl1511 | 12 | 1.173593 | 0.084735 | 4.635733 |
| 7000196649 | pcg0007_39-ncgl0767 | 18 | 1.128663 | 0.008043 | 4.022545 |
| 7000196650 | pcg0007_39-ncgl1262 | 19 | 1.127885 | 0.005913 | 3.95088 |
| 7000196651 | pcg0007_39-ncgl1511 | 7 | 1.119959 | 0.018691 | 3.220332 |
| 7000196657 | pcg0007_39-ncgl0767 | 8 | 1.11792 | 0.008145 | 3.032404 |
| 7000196668 | pcg0007_39-ncgl0767 | 18 | 1.114954 | 0.005466 | 3.439875 |
| 7000196677 | pcg0007_39-ncgl1262 | 14 | 1.131585 | 0.004536 | 4.982825 |
| 7000196687 | pcg0007_39-cg1486 | 20 | 1.129246 | 0.004393 | 4.76576 |
| 7000196703 | pcg0007_39-ncgl1262 | 20 | 1.17434 | 0.003597 | 8.949436 |
| 7000197878 | pcg0007_39-ncgl0304 | 16 | 1.106952 | 0.005627 | 3.246974 |
| 7000197883 | pcg0007_39-ncgl1262 | 19 | 1.129494 | 0.00609 | 5.076771 |
| 7000197896 | pcg0007_39-ncgl0767 | 22 | 1.199136 | 0.005992 | 5.506654 |
| 7000197934 | pcg0007_39-ncgl1262 | 20 | 1.112516 | 0.005336 | 7.136187 |

As shown in Table 9, off-pathway target genes that exhibit a significant increase in lysine production when operably linked to a heterologous promoter exhibit an overrepresentation of certain GOSlim terms.

TABLE 9
Recombinant strains of C. glutamicum having modified expression of non-L-lysine Biosynthetic
Genes and yield change from base of at least 3%, and associated GOSlim terms

| promoter- | | Improvement | |
|---|---|---|---|
| target | N | Over Parent | GOSlims |
| pcg1860-pfka | 1 | 4.39028632 | GO:0051186; GO:0005975; GO:0016301; GO:0043167; GO:0034641; |
| | | | GO:0008150; GO:0044281; GO:0003674; GO:0006091; GO:0009056 |
| pcg0007_265- | 1 | 3.34890197 | GO:0051186; GO:0005975; GO:0016301; GO:0043167; GO:0034641; |
| pfka | | | GO:0008150; GO:0044281; GO:0003674; GO:0006091; GO:0009056 |
| pcg3381-pfka | 1 | 11.3980559 | GO:0051186; GO:0005975; GO:0016301; GO:0043167; GO:0034641; |
| | | | GO:0008150; GO:0044281; GO:0003674; GO:0006091; GO:0009056 |
| pcg0007_39- | 6 | 14.4061592 | GO:0008150; GO:0006457; GO:0003674; GO:0051082; GO:0043167 |
| dnak | | | |
| pcg0007-rho | 1 | 13.4898058 | GO:0009058; GO:0008150; GO:0003723; GO:0016887; GO:0003674; |
| | | | GO:0043167; GO:0034641; GO:0004386 |
| pcg0007- | 1 | 12.5839916 | GO:0006457; GO:0003674; GO:0008150; GO:0043167 |
| groel | | | |
| pcg1860-rho | 3 | 14.1730443 | GO:0009058; GO:0008150; GO:0003723; GO:0016887; GO:0003674; |
| | | | GO:0043167; GO:0034641; GO:0004386 |
| pcg1860- | 2 | 13.5818163 | GO:0004518; GO:0016829; GO:0006950; GO:0016798; GO:0034641; |
| mutm2_2522 | | | GO:0008150; GO:0003677; GO:0006259; GO:0003674; GO:0043167 |
| pcg0007_265- | 1 | 13.2635149 | GO:0043167; GO:0003674; GO:0004386 |
| rhle_609 | | | |
| pcg3381-gpsi | 1 | 13.1192293 | GO:0016779; GO:0004518; GO:0043167; GO:0008150; GO:0034641; |
| | | | GO:0003674; GO:0003723; GO:0034655; GO:0009056 |
| pcg3121-rho | 1 | 12.3280267 | GO:0009058; GO:0008150; GO:0003723; GO:0016887; GO:0003674; |
| | | | GO:0043167; GO:0034641; GO:0004386 |
| pcg0007_39- | 5 | 12.4802928 | GO:0003674 |
| cg0074 | | | |
| pcg0007_39- | 2 | 13.8438588 | GO:0008150; GO:0016779; GO:0003674; GO:0043167 |
| glne | | | |
| pcg0007- | 1 | 12.1722755 | GO:0003674 |
| cg0074 | | | |
| pcg0007-glne | 1 | 14.1530559 | GO:0008150; GO:0016779; GO:0003674; GO:0043167 |
| pcg0007_265- | 1 | 12.5857278 | GO:0003674 |
| cg0074 | | | |
| pcg0007_265- | 1 | 17.1431663 | GO:0008150; GO:0016779; GO:0003674; GO:0043167 |
| glne | | | |
| pcg3381- | 1 | 12.266976 | GO:0003674 |
| cg0074 | | | |
| pcg3121- | 7 | 12.8849506 | GO:0003674 |
| cg0074 | | | |
| pcg3121-glne | 2 | 14.2546395 | GO:0008150; GO:0016779; GO:0003674; GO:0043167 |
| pcg1860- | 7 | 8.87000429 | GO:0043167; GO:0003674; GO:0004386 |
| rhle_609 | | | |
| pcg1860- | 7 | 3.51838501 | GO:0043167; GO:0003674; GO:0004386 |
| rhle_609 | | | |
| pcg0007_39- | 4 | −4.5845813 | GO:0009058; GO:0008150; GO:0044281; GO:0003674; GO:0016301; |
| prsa | | | GO:0034641; GO:0043167 |
| pcg0007_39- | 3 | 3.06020314 | GO:0034641; GO:0008150; GO:0001071; GO:0003677; GO:0003674; |
| cg2942 | | | GO:0009058 |
| pcg0007_39- | 61 | −0.6395119 | GO:0003674; GO:0003677 |
| cg1527 | | | |
| pcg0007_39- | 4 | 0.76168482 | GO:0034641; GO:0003674; GO:0001071; GO:0003677; GO:0008150; |
| cg3239 | | | GO:0009058 |
| pcg1860- | 1 | −0.5888931 | GO:0034641; GO:0003674; GO:0001071; GO:0003677; GO:0008150; |
| cg3239 | | | GO:0009058 |
| pcg0007_39- | 3 | −3.8426999 | GO:0034641; GO:0008150; GO:0044281; GO:0003677; GO:0003674; |
| cg2831_2140 | | | GO:0009058 |
| pcg1860- | 1 | −7.8963623 | GO:0034641; GO:0003674; GO:0003723; GO:0008150; GO:0016829; |
| cg1615 | | | GO:0016853 |
| pcg0007_39- | 4 | −3.1295926 | GO:0044403; GO:0006950; GO:0008150 |
| cg2151_1597 | | | |
| pcg1860- | 1 | −7.3331102 | GO:0044403; GO:0006950; GO:0008150 |
| cg2151_1597 | | | |
| pcg1860- | 1 | −3.2598098 | GO:0034641; GO:0003674; GO:0001071; GO:0003677; GO:0008150; |
| cg2783_2117 | | | GO:0009058 |
| pcg0007_39- | 2 | −6.1189202 | GO:0003674; GO:0001071; GO:0003677; GO:0008150; GO:0043167 |
| cg2784 | | | |
| pcg0007_39- | 1 | −5.1371168 | GO:0009058; GO:0034641; GO:0003674; GO:0003677; GO:0008150; |
| rpob_384 | | | GO:0016779 |
| pcg1860-ptsi | 1 | −12.991654 | GO:0016301; GO:0003674; GO:0008150; GO:0006810; GO:0043167 |
| pcg0007_39- | 3 | −7.9173341 | GO:0009058; GO:0003674; GO:0008150; GO:0016779 |
| galu2 | | | |
| pcg0007_39- | 9 | 3.92098179 | GO:0034641; GO:0003674; GO:0001071; GO:0003677; GO:0008150; |
| cg2899_2194 | | | GO:0009058 |
| pcg0007_39- | 1 | 4.35468317 | GO:0034641; GO:0008150; GO:0003677; GO:0003674; GO:0009058 |
| cg0646_412 | | | |
| pcg1860- | 1 | 3.73948719 | GO:0034641; GO:0008150; GO:0003677; GO:0003674; GO:0009058 |
| cg0646_412 | | | |
| pcg0007_39- | 3 | 3.82606325 | GO:0034641; GO:0008150; GO:0003677; GO:0003674; GO:0009058 |
| cg1410 | | | |
| pcg0007_39- | 32 | 3.74929273 | GO:0034641; GO:0008150; GO:0003677; GO:0003674; GO:0009058 |
| cg1486 | | | |
| pcg0007_39- | 45 | 5.47188771 | GO:0034641; GO:0003674; GO:0001071; GO:0003677; GO:0008150; |
| cg2766 | | | GO:0009058 |
| pcg1860- | 1 | 3.01149306 | GO:0034641; GO:0003674; GO:0001071; GO:0003677; GO:0008150; |
| cg0537 | | | GO:0009058 |
| pcg0007_39- | 3 | 3.09596969 | GO:0034641; GO:0008150; GO:0003677; GO:0003674; GO:0009058 |
| hrca | | | |
| pcg1860- | 1 | 3.74501997 | GO:0034641; GO:0008150; GO:0001071; GO:0003677; GO:0003674; |
| cg2965 | | | GO:0009058 |
| pcg0007_39- | 1 | 3.71827755 | GO:0034641; GO:0003674; GO:0001071; GO:0003677; GO:0008150; |
| cg0702_459 | | | GO:0009058 |
| pcg0007_39- | 2 | 3.55921201 | GO:0034641; GO:0003674; GO:0001071; GO:0003677; GO:0008150; |
| cg0702_460 | | | GO:0009058 |
| pcg0007_39- | 2 | −6.7755339 | GO:0034641; GO:0003674; GO:0001071; GO:0003677; GO:0008150; |
| cg1324_966 | | | GO:0009058 |
| pcg0007_39- | 5 | 3.57846953 | GO:0034641; GO:0003674; GO:0044281; GO:0008150; GO:0016301; |
| cmk | | | GO:0043167 |
| pcg1860-cmk | 1 | 4.52929794 | GO:0034641; GO:0003674; GO:0044281; GO:0008150; GO:0016301; |
| | | | GO:0043167 |
| pcg0007_39- | 52 | −2.3418417 | GO:0009058; GO:0008150; GO:0003723; GO:0016887; GO:0003674; |
| rho | | | GO:0043167; GO:0034641; GO:0004386 |
| pcg0007_39- | 59 | 3.51774505 | GO:0009058; GO:0034641; GO:0003674; GO:0003677; GO:0008150; |
| rpob_383 | | | GO:0016779 |
| pcg0007_39- | 39 | 4.68692368 | GO:0071554; GO:0009058; GO:0003674; GO:0008150; GO:0016874; |
| ddl | | | GO:0043167 |
| pcg0007_39- | 26 | 5.00884445 | GO:0003674; GO:0008150; GO:0003677 |
| cg0027 | | | |
| pcg0007_39- | 39 | 3.26031674 | GO:0071554; GO:0009058; GO:0003674; GO:0008150; GO:0016874; |
| ddl | | | GO:0043167 |
| pcg0007_39- | 59 | 5.35251645 | GO:0009058; GO:0034641; GO:0003674; GO:0003677; GO:0008150; |
| rpob_383 | | | GO:0016779 |
| pcg0007_39- | 59 | 6.12355761 | GO:0009058; GO:0034641; GO:0003674; GO:0003677; GO:0008150; |
| rpob_383 | | | GO:0016779 |
| pcg0007_39- | 26 | 9.94448777 | GO:0003674; GO:0008150; GO:0003677 |
| cg0027 | | | |
| pcg0007_39- | 39 | −2.1858149 | GO:0071554; GO:0009058; GO:0003674; GO:0008150; GO:0016874; |
| ddl | | | GO:0043167 |
| pcg0007_39- | 41 | 5.89061417 | GO:0034641; GO:0003674; GO:0001071; GO:0003677; GO:0008150; |
| cg0725 | | | GO:0009058 |
| pcg0007_39- | 26 | 5.81485995 | GO:0003674; GO:0008150; GO:0003677 |
| cg0027 | | | |
| pcg0007_39- | 61 | 6.67645276 | GO:0003674; GO:0003677 |
| cg1527 | | | |
| pcg0007_39- | 39 | 5.92518842 | GO:0071554; GO:0009058; GO:0003674; GO:0008150; GO:0016874; |
| ddl | | | GO:0043167 |
| pcg0007_39- | 59 | 6.67247895 | GO:0009058; GO:0034641; GO:0003674; GO:0003677; GO:0008150; |
| rpob_383 | | | GO:0016779 |
| pcg0007_39- | 41 | 3.23170311 | GO:0034641; GO:0003674; GO:0001071; GO:0003677; GO:0008150; |
| cg0725 | | | GO:0009058 |
| pcg0007_39- | 41 | 6.41111591 | GO:0034641; GO:0003674; GO:0001071; GO:0003677; GO:0008150; |
| cg0725 | | | GO:0009058 |
| pcg0007_39- | 39 | 3.36382174 | GO:0071554; GO:0009058; GO:0003674; GO:0008150; GO:0016874; |
| ddl | | | GO:0043167 |
| pcg0007_39- | 41 | 11.8263514 | GO:0034641; GO:0003674; GO:0001071; GO:0003677; GO:0008150; |
| cg0725 | | | GO:0009058 |
| pcg0007_39- | 45 | 13.0573003 | GO:0034641; GO:0003674; GO:0001071; GO:0003677; GO:0008150; |
| cg2766 | | | GO:0009058 |
| pcg0007_39- | 41 | 3.17383831 | GO:0034641; GO:0003674; GO:0001071; GO:0003677; GO:0008150; |
| cg0725 | | | GO:0009058 |
| pcg0007_39- | 2 | 3.80588976 | GO:0034641; GO:0003674; GO:0001071; GO:0003677; GO:0008150; |
| cg3315 | | | GO:0009058 |
| pcg0007_39- | 5 | 4.88364585 | GO:0003674; GO:0008150; GO:0003677 |
| hspr | | | |
| pcg0007_39- | 4 | 3.39617637 | GO:0034641; GO:0008150; GO:0003677; GO:0003674; GO:0009058 |
| cg2910 | | | |
| pcg0007_39- | 5 | 5.137602 | GO:0034641; GO:0008150; GO:0003677; GO:0003674; GO:0009058 |
| cg3352 | | | |
| pcg0007_39- | 2 | 5.60015218 | GO:0003674 |
| cg2177_1627 | | | |
| pcg0007_39- | 4 | 4.89731116 | GO:0008150; GO:0016746; GO:0003674 |
| plsc_1822 | | | |
| pcg0007_39- | 9 | 5.87780715 | GO:0034641; GO:0003674; GO:0001071; GO:0003677; GO:0008150; |
| cg2899_2194 | | | GO:0009058 |
| pcg0007_39- | 4 | 2.27062338 | GO:0034641; GO:0008150; GO:0009058 |
| nusg_374 | | | |
| pcg0007_39- | 4 | 4.03184061 | GO:0034641; GO:0008150; GO:0003677; GO:0003674; GO:0009058 |
| cg0646_413 | | | |
| pcg0007_39- | 5 | 2.89398198 | GO:0003674; GO:0008150; GO:0016887 |
| para2_1175 | | | |
| pcg0007_39- | 4 | 3.31061965 | GO:0044403; GO:0006950; GO:0008150 |
| cg2151_1597 | | | |
| pcg0007_39- | 45 | 6.26071191 | GO:0034641; GO:0003674; GO:0001071; GO:0003677; GO:0008150; |
| cg2766 | | | GO:0009058 |
| pcg0007_39- | 45 | 6.31881824 | GO:0034641; GO:0003674; GO:0001071; GO:0003677; GO:0008150; |
| cg2766 | | | GO:0009058 |
| pcg0007_39- | 34 | 3.10053677 | GO:0034641; GO:0008150; GO:0001071; GO:0003677; GO:0003674; |
| cg2965 | | | GO:0009058 |
| pcg0007_39- | 59 | 4.88737996 | GO:0009058; GO:0034641; GO:0003674; GO:0003677; GO:0008150; |
| rpob_383 | | | GO:0016779 |
| pcg0007_39- | 45 | 5.14282462 | GO:0034641; GO:0003674; GO:0001071; GO:0003677; GO:0008150; |
| cg2766 | | | GO:0009058 |
| pcg0007_39- | 45 | 3.95776653 | GO:0034641; GO:0003674; GO:0001071; GO:0003677; GO:0008150; |
| cg2766 | | | GO:0009058 |
| pcg0007_39- | 9 | 3.18608122 | GO:0034641; GO:0003674; GO:0001071; GO:0003677; GO:0008150; |
| cg2899_2194 | | | GO:0009058 |
| pcg0007_39- | 52 | 1.78153221 | GO:0009058; GO:0008150; GO:0003723; GO:0016887; GO:0003674; |
| rho | | | GO:0043167; GO:0034641; GO:0004386 |
| pcg0007_39- | 5 | 4.45053021 | GO:0003674 |
| cg0074 | | | |
| pcg3121- | 7 | 3.31115349 | GO:0003674 |
| cg0074 | | | |
| pcg0007_39- | 4 | −1.0859037 | GO:0004518; GO:0003674 |
| cg2453 | | | |
| pcg3121- | 35 | −0.238969 | GO:0004518; GO:0016829; GO:0006950; GO:0016798; GO:0034641; |
| mutm2_2522 | | | GO:0008150; GO:0003677; GO:0006259; GO:0003674; GO:0043167 |
| pcg0007_39- | 45 | 4.19666701 | GO:0034641; GO:0003674; GO:0001071; GO:0003677; GO:0008150; |
| cg2766 | | | GO:0009058 |
| pcg0007_39- | 45 | 0.57574968 | GO:0034641; GO:0003674; GO:0001071; GO:0003677; GO:0008150; |
| cg2766 | | | GO:0009058 |
| pcg0007_39- | 52 | 0.87040444 | GO:0009058; GO:0008150; GO:0003723; GO:0016887; GO:0003674; |
| rho | | | GO:0043167; GO:0034641; GO:0004386 |
| pcg0007_39- | 45 | 3.66027494 | GO:0034641; GO:0003674; GO:0001071; GO:0003677; GO:0008150; |
| cg2766 | | | GO:0009058 |
| pcg0007_39- | 45 | 12.6184078 | GO:0034641; GO:0003674; GO:0001071; GO:0003677; GO:0008150; |
| cg2766 | | | GO:0009058 |
| pcg0007_39- | 51 | 13.6638701 | GO:0034641; GO:0003674; GO:0001071; GO:0003677; GO:0008150; |
| cg2899 | | | GO:0009058 |
| pcg0007_39- | 52 | 10.9737942 | GO:0009058; GO:0008150; GO:0003723; GO:0016887; GO:0003674; |
| rho | | | GO:0043167; GO:0034641; GO:0004386 |
| pcg0007_39- | 45 | 4.09238291 | GO:0034641; GO:0003674; GO:0001071; GO:0003677; GO:0008150; |
| cg2766 | | | GO:0009058 |
| pcg0007_39- | 45 | 7.29195777 | GO:0034641; GO:0003674; GO:0001071; GO:0003677; GO:0008150; |
| cg2766 | | | GO:0009058 |
| pcg0007_39- | 41 | 8.62140196 | GO:0034641; GO:0003674; GO:0001071; GO:0003677; GO:0008150; |
| cg0725 | | | GO:0009058 |
| pcg0007_39- | 45 | 3.19043193 | GO:0034641; GO:0003674; GO:0001071; GO:0003677; GO:0008150; |
| cg2766 | | | GO:0009058 |
| pcg0007_39- | 45 | 1.62135831 | GO:0034641; GO:0003674; GO:0001071; GO:0003677; GO:0008150; |
| cg2766 | | | GO:0009058 |
| pcg0007_39- | 45 | 5.54788425 | GO:0034641; GO:0003674; GO:0001071; GO:0003677; GO:0008150; |
| cg2766 | | | GO:0009058 |
| pcg0007_39- | 61 | −0.9105898 | GO:0003674; GO:0003677 |
| cg1527 | | | |
| pcg1860-xerd | 32 | −1.0306474 | GO:0034641; GO:0008150; GO:0007049; GO:0003677; GO:0006259; |
| | | | GO:0003674; GO:0007059; GO:0032196; GO:0051301 |
| pcg0007_39- | 44 | 4.59950374 | GO:0034641; GO:0003674; GO:0001071; GO:0003677; GO:0008150; |
| urer | | | GO:0009058 |
| pcg1860-xerd | 32 | 0.51774094 | GO:0034641; GO:0008150; GO:0007049; GO:0003677; GO:0006259; |
| | | | GO:0003674; GO:0007059; GO:0032196; GO:0051301 |
| pcg0007_39- | 52 | 0.80226785 | GO:0034641; GO:0008150; GO:0009058 |
| nusg | | | |
| pcg0007_39- | 52 | 5.6808904 | GO:0034641; GO:0008150; GO:0009058 |
| nusg | | | |
| pcg3121- | 35 | 4.53191788 | GO:0004518; GO:0016829; GO:0006950; GO:0016798; GO:0034641; |
| mutm2_2522 | | | GO:0008150; GO:0003677; GO:0006259; GO:0003674; GO:0043167 |
| pcg1860-xerd | 32 | 0.45401159 | GO:0034641; GO:0008150; GO:0007049; GO:0003677; GO:0006259; |
| | | | GO:0003674; GO:0007059; GO:0032196; GO:0051301 |
| pcg0007_39- | 39 | 3.73244268 | GO:0071554; GO:0009058; GO:0003674; GO:0008150; GO:0016874; |
| ddl | | | GO:0043167 |
| pcg0007_39- | 51 | 6.19844239 | GO:0034641; GO:0003674; GO:0001071; GO:0003677; GO:0008150; |
| cg2899 | | | GO:0009058 |
| pcg0007_39- | 34 | 9.37135808 | GO:0034641; GO:0008150; GO:0001071; GO:0003677; GO:0003674; |
| cg2965 | | | GO:0009058 |
| pcg0007_39- | 39 | 11.6387877 | GO:0071554; GO:0009058; GO:0003674; GO:0008150; GO:0016874; |
| ddl | | | GO:0043167 |
| pcg3121- | 35 | 14.9412244 | GO:0004518; GO:0016829; GO:0006950; GO:0016798; GO:0034641; |
| mutm2_2522 | | | GO:0008150; GO:0003677; GO:0006259; GO:0003674; GO:0043167 |
| pcg0007_39- | 51 | 0.8074962 | GO:0034641; GO:0003674; GO:0001071; GO:0003677; GO:0008150; |
| cg2899 | | | GO:0009058 |
| pcg0007_39- | 34 | −0.9759507 | GO:0034641; GO:0008150; GO:0001071; GO:0003677; GO:0003674; |
| cg2965 | | | GO:0009058 |
| pcg0007_39- | 39 | −0.1918499 | GO:0071554; GO:0009058; GO:0003674; GO:0008150; GO:0016874; |
| ddl | | | GO:0043167 |
| pcg3121- | 35 | 0.62085394 | GO:0004518; GO:0016829; GO:0006950; GO:0016798; GO:0034641; |
| mutm2_2522 | | | GO:0008150; GO:0003677; GO:0006259; GO:0003674; GO:0043167 |
| pcg0007_39- | 45 | 5.03827938 | GO:0034641; GO:0003674; GO:0001071; GO:0003677; GO:0008150; |
| cg2766 | | | GO:0009058 |
| pcg0007_39- | 45 | 4.46262469 | GO:0034641; GO:0003674; GO:0001071; GO:0003677; GO:0008150; |
| cg2766 | | | GO:0009058 |
| pcg0007_39- | 45 | 5.34727763 | GO:0034641; GO:0003674; GO:0001071; GO:0003677; GO:0008150; |
| cg2766 | | | GO:0009058 |
| pcg0007_39- | 51 | 0.8195123 | GO:0034641; GO:0003674; GO:0001071; GO:0003677; GO:0008150; |
| cg2899 | | | GO:0009058 |
| pcg0007_39- | 61 | 0.7420368 | GO:0003674; GO:0003677 |
| cg1527 | | | |
| pcg0007_39- | 34 | −0.9362595 | GO:0034641; GO:0008150; GO:0001071; GO:0003677; GO:0003674; |
| cg2965 | | | GO:0009058 |
| pcg0007_39- | 45 | 3.65582546 | GO:0034641; GO:0003674; GO:0001071; GO:0003677; GO:0008150; |
| cg2766 | | | GO:0009058 |
| pcg3381- | 10 | 0.10953454 | GO:0034641; GO:0008150; GO:0003677; GO:0003674; GO:0009058 |
| cg1410 | | | |
| pcg3121- | 1 | 3.04229587 | GO:0034641; GO:0003674; GO:0001071; GO:0003677; GO:0008150; |
| cg2783_2117 | | | GO:0009058 |
| pcg0007_39- | 45 | 2.64354688 | GO:0034641; GO:0003674; GO:0001071; GO:0003677; GO:0008150; |
| cg2766 | | | GO:0009058 |
| pcg1860-xerd | 32 | −2.7110091 | GO:0034641; GO:0008150; GO:0007049; GO:0003677; GO:0006259; |
| | | | GO:0003674; GO:0007059; GO:0032196; GO:0051301 |
| pcg0007_39- | 59 | −0.290262 | GO:0009058; GO:0034641; GO:0003674; GO:0003677; GO:0008150; |
| rpob_383 | | | GO:0016779 |
| pcg0007_39- | 45 | 5.01151095 | GO:0034641; GO:0003674; GO:0001071; GO:0003677; GO:0008150; |
| cg2766 | | | GO:0009058 |
| pcg0007_39- | 45 | 5.41102769 | GO:0034641; GO:0003674; GO:0001071; GO:0003677; GO:0008150; |
| cg2766 | | | GO:0009058 |
| pcg0007_39- | 3 | 0.45383689 | GO:0034641; GO:0003674; GO:0001071; GO:0008150; GO:0009058 |
| cg3082_2321 | | | |
| pcg0007_39- | 9 | 0.66363765 | GO:0034641; GO:0003674; GO:0001071; GO:0003677; GO:0008150; |
| cg2899_2194 | | | GO:0009058 |
| pcg0007_39- | 23 | 3.70271695 | GO:0009058; GO:0008150; GO:0044281; GO:0003674; GO:0006520; |
| tyra | | | GO:0016491 |
| pcg0007_39- | 32 | 4.13440802 | GO:0034641; GO:0008150; GO:0003677; GO:0003674; GO:0009058 |
| cg1486 | | | |
| pcg0007_39- | 44 | −1.2304485 | GO:0008150; GO:0003674; GO:0003677 |
| cg0800_539 | | | |
| pcg0007_39- | 45 | 2.65554177 | GO:0034641; GO:0003674; GO:0001071; GO:0003677; GO:0008150; |
| cg2766 | | | GO:0009058 |
| pcg0007_39- | 52 | −2.294756 | GO:0009058; GO:0008150; GO:0003723; GO:0016887; GO:0003674; |
| rho | | | GO:0043167; GO:0034641; GO:0004386 |
| pcg0007_39- | 44 | 0.72184473 | GO:0034641; GO:0003674; GO:0001071; GO:0003677; GO:0008150; |
| urer | | | GO:0009058 |
| pcg1860-xerd | 32 | 0.06000601 | GO:0034641; GO:0008150; GO:0007049; GO:0003677; GO:0006259; |
| | | | GO:0003674; GO:0007059; GO:0032196; GO:0051301 |
| pcg0007_39- | 51 | 3.72132191 | GO:0034641; GO:0003674; GO:0001071; GO:0003677; GO:0008150; |
| cg2899 | | | GO:0009058 |
| pcg0007_39- | 26 | 3.51965405 | GO:0003674; GO:0008150; GO:0003677 |
| cg0027 | | | |
| pcg0007_39- | 26 | 1.29180691 | GO:0003674; GO:0008150; GO:0003677 |
| cg0027 | | | |
| pcg0007_39- | 1 | −17.04716 | GO:0005975; GO:0009058; GO:0008150; GO:0016757; GO:0003674 |
| ncgl2535 | | | |
| pcg0007_39- | 1 | 0.34018726 | GO:0051186; GO:0009058; GO:0043167; GO:0034641; GO:0003674; |
| ncgl2446 | | | GO:0044281; GO:0008150; GO:0016874 |
| pcg0007_39- | 1 | −0.3317848 | GO:0008150; GO:0009058; GO:0003674; GO:0006629 |
| ncgl0600 | | | |
| pcg0007_39- | 5 | 0.41840795 | GO:0034641; GO:0008150; GO:0044281; GO:0003674; GO:0016810; |
| ncgl0827 | | | GO:0009058 |
| pcg0007_39- | 17 | 0.56929624 | GO:0016829; GO:0009058; GO:0034641; GO:0008150; GO:0044281; |
| ncgl0306 | | | GO:0004871; GO:0003674; GO:0007165 |
| pcg0007_39- | 11 | −0.388723 | GO:0034641; GO:0003674; GO:0044281; GO:0008150; GO:0016301; |
| ncgl1948 | | | GO:0009058; GO:0043167 |
| pcg0007_39- | 1 | 0.57425156 | GO:0003674; GO:0051186; GO:0009058; GO:0043167; GO:0016765; |
| ncgl1961 | | | GO:0034641; GO:0008150; GO:0044281; GO:0006790 |
| pcg0007_39- | 1 | −2.6435068 | GO:0034641; GO:0008150; GO:0044281; GO:0003674; GO:0016874; |
| ncgl2669 | | | GO:0009058; GO:0043167 |
| pcg0007_39- | 1 | −2.1326692 | GO:0051186; GO:0009058; GO:0034641; GO:0003674; GO:0008150; |
| ncgl1508 | | | GO:0016491 |
| pcg0007_39- | 1 | −1.7218114 | GO:0051186; GO:0009058; GO:0022607; GO:0006790; GO:0008150 |
| ncgl1503 | | | |
| pcg0007_39- | 15 | 3.48436935 | GO:0051186; GO:0009058; GO:0016765; GO:0034641; GO:0008150; |
| ncgl1511 | | | GO:0003674 |
| pcg0007_39- | 2 | −1.3907926 | GO:0016765; GO:0009058; GO:0008150; GO:0006629; GO:0003674; |
| ncgl0598 | | | GO:0044281 |
| pcg0007_39- | 1 | −0.0604086 | GO:0009058; GO:0008150; GO:0006629; GO:0003674; GO:0016829; |
| ncgl2569 | | | GO:0044281; GO:0043167 |
| pcg0007_39- | 1 | 0.97131028 | GO:0034641; GO:0051186; GO:0008150; GO:0003674; GO:0019748; |
| ncgl1905 | | | GO:0009058; GO:0043167 |
| pcg0007_39- | 1 | −0.5809877 | GO:0034641; GO:0003674; GO:0044281; GO:0008150; GO:0016301; |
| ncgl2287 | | | GO:0009058; GO:0043167 |
| pcg0007_39- | 1 | 0.29165558 | GO:0009058; GO:0051186; GO:0006790; GO:0008150; GO:0043167; |
| ncgl1457 | | | GO:0003674; GO:0016874 |
| pcg0007_39- | 1 | −3.5394984 | GO:0043167; GO:0009058; GO:0008150; GO:0006629; GO:0003674; |
| ncgl0874 | | | GO:0016301; GO:0044281 |
| pcg0007_39- | 1 | −1.532018 | GO:0042592; GO:0003674; GO:0008150; GO:0043167; GO:0016491 |
| ncgl1928 | | | |
| pcg0007_39- | 1 | −3.372779 | GO:0006950; GO:0043167; GO:0034641; GO:0003674; GO:0016887; |
| ncgl1880 | | | GO:0003677; GO:0006259; GO:0008150 |
| pcg0007_39- | 2 | −3.3625411 | GO:0003674; GO:0008150; GO:0016491 |
| ncgl0663 | | | |
| pcg0007_39- | 1 | −1.659528 | GO:0004518; GO:0016829; GO:0006950; GO:0016798; GO:0034641; |
| ncgl0813 | | | GO:0008150; GO:0003677; GO:0006259; GO:0003674; GO:0043167 |
| pcg0007_39- | 1 | −2.0046229 | GO:0003674; GO:0008150; GO:0016491 |
| ncgl2286 | | | |
| pcg0007_39- | 2 | −2.6375612 | GO:0004518; GO:0034641; GO:0008150; GO:0006259; GO:0003674; |
| ncgl0641 | | | GO:0006950 |
| pcg0007_39- | 1 | −1.1543868 | GO:0006457; GO:0034641; GO:0051082; GO:0043167; GO:0009058; |
| ncgl2210 | | | GO:0006950; GO:0008150; GO:0006259; GO:0003674 |
| pcg0007_39- | 7 | −1.6400734 | GO:0034641; GO:0008150; GO:0008168; GO:0006259; GO:0003674; |
| ncgl2901 | | | GO:0006950 |
| pcg0007_39- | 7 | −3.1981371 | GO:0003674; GO:0016853; GO:0008150; GO:0016491; GO:0043167 |
| ncgl0877 | | | |
| pcg0007_39- | 1 | −3.302181 | GO:0006950; GO:0003674; GO:0008150; GO:0016491 |
| ncgl2984 | | | |
| pcg0007_39- | 1 | −3.0186755 | GO:0034641; GO:0009058; GO:0006950; GO:0008150; GO:0003677; |
| ncgl1855 | | | GO:0006259; GO:0003674; GO:0008233 |
| pcg0007_39- | 6 | −2.9642887 | GO:0004518; GO:0006950; GO:0043167; GO:0034641; GO:0003674; |
| ncgl1322 | | | GO:0016887; GO:0003677; GO:0006259; GO:0008150 |
| pcg0007_39- | 1 | −1.8831379 | GO:0022607; GO:0006461; GO:0065003; GO:0008150; GO:0003674 |
| ncgl0427 | | | |
| pcg0007_39- | 1 | −2.4009195 | GO:0006950; GO:0034641; GO:0043167; GO:0009058; GO:0008150; |
| ncgl1196 | | | GO:0006259; GO:0003674; GO:0016874 |
| pcg0007_39- | 1 | −2.286499 | GO:0016779; GO:0034641; GO:0043167; GO:0006950; GO:0003674; |
| ncgl2576 | | | GO:0007165; GO:0003677; GO:0006259; GO:0008150 |
| pcg0007_39- | 2 | −0.4397465 | GO:0042592; GO:0003674; GO:0016853; GO:0008150; GO:0016491 |
| ncgl0424 | | | |
| pcg0007_39- | 1 | −0.6772859 | GO:0034641; GO:0008150; GO:0006259; GO:0006950 |
| ncgl2204 | | | |
| pcg0007_39- | 1 | −0.2836098 | GO:0022607; GO:0006461; GO:0065003; GO:0008150 |
| ncgl0425 | | | |
| pcg0007_39- | 1 | −9.9973443 | GO:0006950; GO:0008150 |
| ncgl2755 | | | |
| pcg0007_39- | 1 | −0.9815224 | GO:0006950; GO:0008150; GO:0006259; GO:0003674; GO:0034641; |
| ncgl0142 | | | GO:0016798 |
| pcg0007_39- | 2 | −0.3108331 | GO:0006950; GO:0043167; GO:0034641; GO:0008150; GO:0003677; |
| ncgl0241 | | | GO:0006259; GO:0003674 |
| pcg0007_39- | 1 | −2.484144 | GO:0008150; GO:0003674; GO:0016810; GO:0009056; GO:0043167 |
| ncgl2789 | | | |
| pcg0007_39- | 1 | −2.1347389 | GO:0006520; GO:0034641; GO:0043167; GO:0009058; GO:0008150; |
| ncgl2929 | | | GO:0044281; GO:0016757; GO:0003674 |
| pcg0007_39- | 1 | −2.7583354 | GO:0009058; GO:0008150; GO:0044281; GO:0003674; GO:0016829; |
| ncgl0408 | | | GO:0006520 |
| pcg0007_39- | 7 | −2.8766171 | GO:0006520; GO:0003674; GO:0044281; GO:0008150; GO:0016874 |
| ncgl1484 | | | |
| pcg0007_39- | 2 | −2.1343277 | GO:0003674; GO:0016829; GO:0006520; GO:0009058; GO:0008150; |
| ncgl2473 | | | GO:0044281; GO:0006790 |
| pcg0007_39- | 2 | −2.7490574 | GO:0003674; GO:0016829; GO:0006520; GO:0009058; GO:0008150; |
| ncgl2473 | | | GO:0044281; GO:0006790 |
| pcg0007_39- | 1 | −2.7534611 | GO:0005975; GO:0016301; GO:0043167; GO:0008150; GO:0044281; |
| ncgl2790 | | | GO:0003674; GO:0009056 |
| pcg0007_39- | 1 | −0.9048132 | GO:0006520; GO:0016301; GO:0043167; GO:0009058; GO:0008150; |
| ncgl2274 | | | GO:0044281; GO:0003674; GO:0003723 |
| pcg0007_39- | 1 | −1.1695153 | GO:0009058; GO:0003674; GO:0044281; GO:0008150; GO:0006520; |
| ncgl0398 | | | GO:0016491 |
| pcg0007_39- | 16 | 8.26570011 | GO:0016829; GO:0006520; GO:0043167; GO:0009058; GO:0008150; |
| ncgl1262 | | | GO:0044281; GO:0003674 |
| pcg0007_39- | 1 | 0.01729616 | GO:0034641; GO:0008150; GO:0044281; GO:0003674; GO:0006520; |
| ncgl1550 | | | GO:0009058; GO:0043167 |
| pcg0007_39- | 1 | −0.1957523 | GO:0008150; GO:0003674; GO:0005975 |
| ncgl2505 | | | |
| pcg0007_39- | 1 | 0.0396492 | GO:0008150; GO:0003674; GO:0016301; GO:0005975; GO:0043167 |
| ncgl2399 | | | |
| pcg0007_39- | 1 | 0.92749467 | GO:0008150; GO:0044281; GO:0003674; GO:0016829; GO:0006520; |
| ncgl1500 | | | GO:0006790; GO:0043167 |
| pcg0007_39- | 1 | 0.5029595 | GO:0009058; GO:0016301; GO:0008150; GO:0044281; GO:0003674; |
| ncgl1137 | | | GO:0006520; GO:0043167 |
| pcg0007_39- | 1 | −2.0644554 | GO:0006520; GO:0006790; GO:0043167; GO:0009058; GO:0008150; |
| ncgl2048 | | | GO:0044281; GO:0008168; GO:0003674 |
| pcg0007_39- | 1 | 1.24260236 | GO:0016829; GO:0006520; GO:0034641; GO:0009058; GO:0008150; |
| ncgl2019 | | | GO:0044281; GO:0003674 |
| pcg0007_39- | 1 | −0.3679826 | GO:0008150; GO:0044281; GO:0003674; GO:0009056; GO:0005975; |
| ncgl2559 | | | GO:0016853 |
| pcg0007_39- | 1 | −0.8689167 | GO:0008150; GO:0044281; GO:0003674; GO:0006091; GO:0016746; |
| ncgl2247 | | | GO:0005975; GO:0043167 |
| pcg0007_39- | 1 | −1.5472722 | GO:0016779; GO:0008150; GO:0044281; GO:0003674; GO:0005975; |
| ncgl2002 | | | GO:0043167 |
| pcg0007_39- | 1 | −0.5896195 | GO:0008150; GO:0003674; GO:0016301; GO:0005975 |
| ncgl2905 | | | |
| pcg0007_39- | 1 | −0.8260018 | GO:0003674; GO:0008150; GO:0008233; GO:0016746; GO:0034641; |
| ncgl0916 | | | GO:0009056; GO:0006790 |
| pcg0007_39- | 1 | −1.3961923 | GO:0009058; GO:0008150; GO:0044281; GO:0005975; GO:0016829; |
| ncgl1583 | | | GO:0003674 |
| pcg0007_39- | 1 | −0.6491076 | GO:0008150; GO:0003674; GO:0005975; GO:0016798 |
| ncgl0853 | | | |
| pcg0007_39- | 1 | −0.5659838 | GO:0016853; GO:0016829; GO:0006520; GO:0034641; GO:0009058; |
| ncgl2930 | | | GO:0008150; GO:0044281; GO:0003674 |
| pcg0007_39- | 44 | −0.2183031 | GO:0034641; GO:0003674; GO:0001071; GO:0003677; GO:0008150; |
| urer | | | GO:0009058 |
| pcg0007_39- | 44 | 2.15803348 | GO:0034641; GO:0003674; GO:0001071; GO:0003677; GO:0008150; |
| urer | | | GO:0009058 |
| pcg0007_39- | 51 | −0.3543192 | GO:0034641; GO:0003674; GO:0001071; GO:0003677; GO:0008150; |
| cg2899 | | | GO:0009058 |
| pcg0007_39- | 44 | −3.0417686 | GO:0008150; GO:0003674; GO:0003677 |
| cg0800_539 | | | |
| pcg0007_39- | 9 | 5.22190199 | GO:0061024; GO:0006810; GO:0008150 |
| cg3419 | | | |
| pcg0007_39- | 32 | 6.35412587 | GO:0034641; GO:0008150; GO:0003677; GO:0003674; GO:0009058 |
| cg1486 | | | |
| pcg0007_39- | 25 | 3.69092746 | GO:0008150 |
| cg3210 | | | |
| pcg0007_39- | 32 | 5.44090596 | GO:0034641; GO:0008150; GO:0003677; GO:0003674; GO:0009058 |
| cg1486 | | | |
| pcg0007_39- | 44 | 0.86106557 | GO:0034641; GO:0003674; GO:0001071; GO:0003677; GO:0008150; |
| urer | | | GO:0009058 |
| pcg0007_39- | 32 | 3.19973635 | GO:0034641; GO:0008150; GO:0003677; GO:0003674; GO:0009058 |
| cg1486 | | | |
| pcg0007_39- | 25 | 0.28172305 | GO:0008150 |
| cg3210 | | | |
| pcg0007_39- | 44 | 0.40999284 | GO:0008150; GO:0003674; GO:0003677 |
| cg0800_539 | | | |
| pcg0007_39- | 32 | 4.91960389 | GO:0034641; GO:0008150; GO:0003677; GO:0003674; GO:0009058 |
| cg1486 | | | |
| pcg0007_39- | 44 | −1.8148135 | GO:0008150; GO:0003674; GO:0003677 |
| cg0800_539 | | | |
| pcg0007_39- | 32 | 3.11615157 | GO:0034641; GO:0008150; GO:0003677; GO:0003674; GO:0009058 |
| cg1486 | | | |
| pcg0007_39- | 25 | −0.2470034 | GO:0008150 |
| cg3210 | | | |
| pcg0007_39- | 9 | 5.23717007 | GO:0034641; GO:0003674; GO:0003723; GO:0008150; GO:0008135; |
| ncgl0767 | | | GO:0009058; GO:0006412 |
| pcg0007_39- | 7 | 0.32038287 | GO:0006399; GO:0034641; GO:0008150; GO:0003674; GO:0016301 |
| ncgl0564 | | | |
| pcg0007_39- | 2 | 7.04961811 | GO:0006520; GO:0009058; GO:0043167; GO:0006399; GO:0034641; |
| ncgl1607 | | | GO:0008150; GO:0044281; GO:0003674; GO:0016874; GO:0006412 |
| pcg0007_39- | 7 | 4.19660001 | GO:0043167; GO:0006399; GO:0034641; GO:0008150; GO:0003674; |
| ncgl2481 | | | GO:0016491 |
| pcg0007_39- | 8 | 2.21004426 | GO:0034641; GO:0008150; GO:0051276; GO:0003677; GO:0006259; |
| ncgl0304 | | | GO:0003674; GO:0043167; GO:0016853 |
| pcg0007_39- | 10 | 2.20963174 | GO:0034641; GO:0003674; GO:0003723; GO:0008150; GO:0016829; |
| cg1615 | | | GO:0016853 |
| pcg0007_39- | 6 | −2.1901613 | GO:0008150; GO:0016829; GO:0003674 |
| ncgl2491 | | | |
| pcg0007_39- | 21 | −1.225098 | GO:0071554; GO:0009058; GO:0003674; GO:0051301; GO:0008150; |
| murc | | | GO:0016874; GO:0007049; GO:0043167 |
| pcg0007_39- | 25 | 0.75098055 | GO:0008150 |
| cg3210 | | | |
| pcg0007_39- | 32 | 0.92038074 | GO:0034641; GO:0008150; GO:0003677; GO:0003674; GO:0009058 |
| cg1486 | | | |
| pcg0007_39- | 3 | −1.8936532 | GO:0034641; GO:0003674; GO:0001071; GO:0008150; GO:0009058 |
| cg3082 | | | |
| pcg0007_39- | 8 | 0.57521516 | GO:0003674; GO:0016301; GO:0008150; GO:0003677 |
| cg0012 | | | |
| pcg0007_39- | 44 | −1.4559363 | GO:0034641; GO:0003674; GO:0001071; GO:0003677; GO:0008150; |
| urer | | | GO:0009058 |
| pcg0007_39- | 26 | −1.256254 | GO:0003674; GO:0008150; GO:0003677 |
| cg0027 | | | |
| pcg0007_39- | 59 | 0.7674954 | GO:0009058; GO:0034641; GO:0003674; GO:0003677; GO:0008150; |
| rpob_383 | | | GO:0016779 |
| pcg0007_39- | 51 | 1.89896771 | GO:0034641; GO:0003674; GO:0001071; GO:0003677; GO:0008150; |
| cg2899 | | | GO:0009058 |
| pcg0007_39- | 3 | 1.41331708 | GO:0034641; GO:0003674; GO:0001071; GO:0008150; GO:0009058 |
| cg3082 | | | |
| pcg0007_39- | 23 | 3.26417536 | GO:0009058; GO:0008150; GO:0044281; GO:0003674; GO:0006520; |
| tyra | | | GO:0016491 |
| pcg0007_39- | 21 | −0.5881867 | GO:0071554; GO:0009058; GO:0003674; GO:0051301; GO:0008150; |
| murc | | | GO:0016874; GO:0007049; GO:0043167 |
| pcg0007_39- | 44 | −0.0852691 | GO:0008150; GO:0003674; GO:0003677 |
| cg0800_539 | | | |
| pcg0007_39- | 44 | −0.2629748 | GO:0008150; GO:0003674; GO:0003677 |
| cg0800_539 | | | |
| pcg0007_39- | 1 | 4.77198741 | GO:0016746; GO:0003674 |
| ncgl1208 | | | |
| pcg0007_39- | 1 | 3.0707362 | GO:0003674; GO:0008150; GO:0016491 |
| ncgl2449 | | | |
| pcg0007_39- | 2 | 4.12705375 | GO:0008150; GO:0008233; GO:0003674 |
| ncgl2327 | | | |
| pcg0007_39- | 2 | 4.57292995 | GO:0003674 |
| ncgl2250 | | | |
| pcg0007_39- | 3 | 5.38326154 | GO:0006259; GO:0003674; GO:0034641; GO:0008150; GO:0003677 |
| ncgl1545 | | | |
| pcg0007_39- | 32 | 3.03869679 | GO:0034641; GO:0008150; GO:0003677; GO:0003674; GO:0009058 |
| cg1486 | | | |
| pcg0007_39- | 15 | 5.74862357 | GO:0051186; GO:0009058; GO:0016765; GO:0034641; GO:0008150; |
| ncgl1511 | | | GO:0003674 |
| pcg0007_39- | 5 | 4.32642044 | GO:0034641; GO:0008150; GO:0044281; GO:0003674; GO:0016810; |
| ncgl0827 | | | GO:0009058 |
| pcg0007_39- | 11 | −0.2038558 | GO:0034641; GO:0003674; GO:0044281; GO:0008150; GO:0016301; |
| ncgl1948 | | | GO:0009058; GO:0043167 |
| pcg0007_39- | 44 | −0.7937019 | GO:0008150; GO:0003674; GO:0003677 |
| cg0800_539 | | | |
| pcg0007_39- | 23 | 3.25312756 | GO:0009058; GO:0008150; GO:0044281; GO:0003674; GO:0006520; |
| tyra | | | GO:0016491 |
| pcg0007_39- | 32 | 3.14639212 | GO:0034641; GO:0008150; GO:0003677; GO:0003674; GO:0009058 |
| cg1486 | | | |
| pcg0007_39- | 32 | −3.5839145 | GO:0034641; GO:0008150; GO:0003677; GO:0003674; GO:0009058 |
| cg1486 | | | |
| pcg0007_39- | 32 | −1.8163957 | GO:0034641; GO:0008150; GO:0003677; GO:0003674; GO:0009058 |
| cg1486 | | | |
| pcg0007_39- | 23 | −3.5273781 | GO:0009058; GO:0008150; GO:0044281; GO:0003674; GO:0006520; |
| tyra | | | GO:0016491 |
| pcg0007_39- | 15 | −4.7165343 | GO:0016829; GO:0006520; GO:0034641; GO:0009058; GO:0008150; |
| ncgl2931 | | | GO:0044281; GO:0003674 |
| pcg0007_39- | 16 | 7.06792721 | GO:0016829; GO:0006520; GO:0043167; GO:0009058; GO:0008150; |
| ncgl1262 | | | GO:0044281; GO:0003674 |
| pcg0007_39- | 32 | 3.14208716 | GO:0034641; GO:0008150; GO:0003677; GO:0003674; GO:0009058 |
| cg1486 | | | |
| pcg0007_39- | 16 | 5.94884625 | GO:0016829; GO:0006520; GO:0043167; GO:0009058; GO:0008150; |
| ncgl1262 | | | GO:0044281; GO:0003674 |
| pcg0007_39- | 7 | −1.7262637 | GO:0006520; GO:0003674; GO:0044281; GO:0008150; GO:0016874 |
| ncgl1484 | | | |
| pcg0007_39- | 15 | −1.3365563 | GO:0051186; GO:0009058; GO:0016765; GO:0034641; GO:0008150; |
| ncgl1511 | | | GO:0003674 |
| pcg0007_39- | 16 | 5.44104119 | GO:0016829; GO:0006520; GO:0043167; GO:0009058; GO:0008150; |
| ncgl1262 | | | GO:0044281; GO:0003674 |
| pcg0007_39- | 9 | 4.36953707 | GO:0034641; GO:0003674; GO:0003723; GO:0008150; GO:0008135; |
| ncgl0767 | | | GO:0009058; GO:0006412 |
| pcg0007_39- | 4 | −0.3074247 | GO:0034641; GO:0008150; GO:0003677; GO:0003674; GO:0009058 |
| cg2614_2011 | | | |
| pcg0007_39- | 5 | −3.8871473 | GO:0003674; GO:0008150; GO:0003677 |
| hspr | | | |
| pcg0007_39- | 4 | 1.05794426 | GO:0003674; GO:0003677 |
| cg1392_1013 | | | |
| pcg0007_39- | 32 | 2.84208976 | GO:0034641; GO:0008150; GO:0003677; GO:0003674; GO:0009058 |
| cg1486 | | | |
| pcg0007_39- | 3 | 3.6771318 | GO:0003674; GO:0008150; GO:0003677 |
| cg0979_688 | | | |
| pcg0007_39- | 4 | 1.74233214 | GO:0003674 |
| ptsx′ | | | |
| pcg0007_39- | 1 | −0.6808701 | GO:0006810; GO:0008150; GO:0055085 |
| ncgl0546 | | | |
| pcg0007_39- | 2 | −0.264575 | GO:0009058; GO:0008150; GO:0044281; GO:0003674; GO:0006520; |
| ncgl0242 | | | GO:0034641 |
| pcg0007_39- | 1 | 3.52470923 | GO:0009058; GO:0043167; GO:0034641; GO:0008150; GO:0044281; |
| ncgl0578 | | | GO:0003674; GO:0016491 |
| pcg1860-xerd | 32 | 0.29279276 | GO:0034641; GO:0008150; GO:0007049; GO:0003677; GO:0006259; |
| | | | GO:0003674; GO:0007059; GO:0032196; GO:0051301 |
| pcg0007_39- | 7 | −0.8917066 | GO:0006399; GO:0034641; GO:0008150; GO:0003674; GO:0016301 |
| ncgl0564 | | | |
| pcg0007_39- | 7 | −1.1006911 | GO:0034641; GO:0008150; GO:0008168; GO:0006259; GO:0003674; |
| ncgl2901 | | | GO:0006950 |
| pcg0007_39- | 11 | −0.3328713 | GO:0034641; GO:0003674; GO:0044281; GO:0008150; GO:0016301; |
| ncgl1948 | | | GO:0009058; GO:0043167 |
| pcg0007_39- | 7 | −1.1884484 | GO:0008150; GO:0009058; GO:0003674 |
| ncgl1065 | | | |
| pcg0007_39- | 8 | 3.8097718 | GO:0034641; GO:0008150; GO:0051276; GO:0003677; GO:0006259; |
| ncgl0304 | | | GO:0003674; GO:0043167; GO:0016853 |
| pcg0007_39- | 15 | 4.63573323 | GO:0051186; GO:0009058; GO:0016765; GO:0034641; GO:0008150; |
| ncgl1511 | | | GO:0003674 |
| pcg0007_39- | 9 | 4.02254461 | GO:0034641; GO:0003674; GO:0003723; GO:0008150; GO:0008135; |
| ncgl0767 | | | GO:0009058; GO:0006412 |
| pcg0007_39- | 16 | 3.95087992 | GO:0016829; GO:0006520; GO:0043167; GO:0009058; GO:0008150; |
| ncgl1262 | | | GO:0044281; GO:0003674 |
| pcg0007_39- | 15 | 3.22033208 | GO:0051186; GO:0009058; GO:0016765; GO:0034641; GO:0008150; |
| ncgl1511 | | | GO:0003674 |
| pcg0007_39- | 9 | 3.03240412 | GO:0034641; GO:0003674; GO:0003723; GO:0008150; GO:0008135; |
| ncgl0767 | | | GO:0009058; GO:0006412 |
| pcg0007_39- | 9 | 3.43987546 | GO:0034641; GO:0003674; GO:0003723; GO:0008150; GO:0008135; |
| ncgl0767 | | | GO:0009058; GO:0006412 |
| pcg0007_39- | 25 | −0.2786013 | GO:0008150 |
| cg3210 | | | |
| pcg0007_39- | 16 | 4.9828246 | GO:0016829; GO:0006520; GO:0043167; GO:0009058; GO:0008150; |
| ncgl1262 | | | GO:0044281; GO:0003674 |
| pcg0007_39- | 32 | 4.76576015 | GO:0034641; GO:0008150; GO:0003677; GO:0003674; GO:0009058 |
| cg1486 | | | |
| pcg0007_39- | 16 | 8.94943618 | GO:0016829; GO:0006520; GO:0043167; GO:0009058; GO:0008150; |
| ncgl1262 | | | GO:0044281; GO:0003674 |
| pcg0007_39- | 8 | 3.24697423 | GO:0034641; GO:0008150; GO:0051276; GO:0003677; GO:0006259; |
| ncgl0304 | | | GO:0003674; GO:0043167; GO:0016853 |
| pcg0007_39- | 16 | 5.07677112 | GO:0016829; GO:0006520; GO:0043167; GO:0009058; GO:0008150; |
| ncgl1262 | | | GO:0044281; GO:0003674 |
| pcg0007_39- | 25 | 0.46752889 | GO:0008150 |
| cg3210 | | | |
| pcg0007_39- | 7 | 0.00474009 | GO:0008150; GO:0009058; GO:0003674 |
| ncgl1065 | | | |
| pcg0007_39- | 7 | −1.1311402 | GO:0006520; GO:0003674; GO:0044281; GO:0008150; GO:0016874 |
| ncgl1484 | | | |
| pcg0007_39- | 9 | 5.50665417 | GO:0034641; GO:0003674; GO:0003723; GO:0008150; GO:0008135; |
| ncgl0767 | | | GO:0009058; GO:0006412 |
| pcg0007_39- | 16 | 7.1361868 | GO:0016829; GO:0006520; GO:0043167; GO:0009058; GO:0008150; |
| ncgl1262 | | | GO:0044281; GO:0003674 |
| pcg0007_39- | 11 | 2.17493085 | GO:0034641; GO:0003674; GO:0044281; GO:0008150; GO:0016301; |
| ncgl1948 | | | GO:0009058; GO:0043167 |
| pcg0007_39- | 2 | 3.0247232 | GO:0008150; GO:0008233; GO:0003674 |
| ncgl2327 | | | |
| pcg0007_39- | 3 | 5.33577683 | GO:0006259; GO:0003674; GO:0034641; GO:0008150; GO:0003677 |
| ncgl1545 | | | |

### Example 4: Assessing L-Lysine Biosynthesis Modulation by Genes Belonging to Different Shells

Genetic loci across the C. glutamicum genome were tested for potential impact on lysine production by generating strains in which the native promoter regulating the gene's expression was substituted with a promoter selected from SEQ ID NOs: 1-8. The impact of each locus was tested by individually substituting the native promoter with one or more promoters from the promoters defined by SEQ ID NOs: 1-8.

The strains were tested for lysine production in multiple experiments and the mean performance of each strain across all experiments was calculated and compared to a control strain lacking the promoter modification. All strain pairs which differ by a single genetic change were calculated and the difference in lysine production at 96 hours between the strain with the change and the strain without the change was calculated. A change is defined as a hit if this performance difference is significantly greater than 0 (at p=0.01) across all strain pairs that differ by this change.

Table 10 provides each genetic locus/promoter modification combination tested and the performance thereof. Each genetic locus is provided with a shell designation as defined herein. Table 10 provides the locus id of each modified genetic locus, the promoter used to modify expression, whether any strains containing the modification had a significant difference in strain performance, and the shell allocation of the gene associated with the particular locus. As shown in Table 10, by testing the same genetic locus using multiple different promoters, applicants were able to identify loci encoding genes impacting strain performance, including shell 4 genes for which no known relationship with strain performance for lysine production was previously identified (e.g., see rows 49-54).

TABLE 10
Systematic sampling of promoter/target gene combinations from
different target gene shells for biomolecule production

| locus_id | promoter | any_above_threshold | Shell |
|---|---|---|---|
| ncgl0009 | pcg0007_39 | TRUE | 3 |
| ncgl0009 | pcg3121 | FALSE | 3 |
| ncgl0015 | pcg0007_39 | TRUE | 3 |
| ncgl0015 | pcg3381 | FALSE | 3 |
| ncgl0019 | pcg0007_39 | TRUE | 3 |
| ncgl0019 | pcg0755 | FALSE | 3 |
| ncgl0019 | pcg3121 | FALSE | 3 |
| ncgl0019 | pcg3381 | FALSE | 3 |
| ncgl0026 | pcg0007_39 | TRUE | 3 |
| ncgl0031 | pcg0007_39 | FALSE | 3 |
| ncgl0031 | pcg1860 | TRUE | 3 |
| ncgl0031 | pcg3381 | FALSE | 3 |
| ncgl0042 | pcg0007_39 | TRUE | 3 |
| ncgl0042 | pcg1860 | TRUE | 3 |
| ncgl0054 | pcg0007_39 | TRUE | 4 |
| ncgl0054 | pcg3121 | FALSE | 4 |
| ncgl0082 | pcg0007_119 | FALSE | 3 |
| ncgl0082 | pcg0007_39 | TRUE | 3 |
| ncgl0082 | pcg1860 | TRUE | 3 |
| ncgl0082 | pcg3121 | FALSE | 3 |
| ncgl0082 | pcg3381 | FALSE | 3 |
| ncgl0082 | wt | FALSE | 3 |
| ncgl0111 | pcg0007_39 | TRUE | 4 |
| ncgl0167 | pcg0007_39 | TRUE | 3 |
| ncgl0167 | pcg1860 | TRUE | 3 |
| ncgl0167 | pcg3121 | FALSE | 3 |
| ncgl0182 | pcg0007_39 | FALSE | 2 |
| ncgl0182 | pcg1860 | FALSE | 2 |
| ncgl0182 | pcg3121 | TRUE | 2 |
| ncgl0211 | pcg0007_39 | TRUE | 2 |
| ncgl0211 | pcg0007_39 | TRUE | 3 |
| ncgl0211 | pcg3121 | FALSE | 2 |
| ncgl0211 | pcg3121 | FALSE | 3 |
| ncgl0211 | pcg3381 | FALSE | 2 |
| ncgl0211 | pcg3381 | FALSE | 3 |
| ncgl0223 | pcg0007_39 | TRUE | 3 |
| ncgl0223 | pcg0755 | FALSE | 3 |
| ncgl0223 | pcg2613 | FALSE | 3 |
| ncgl0223 | pcg3381 | FALSE | 3 |
| ncgl0253 | pcg0007_39 | TRUE | 3 |
| ncgl0253 | pcg1860 | TRUE | 3 |
| ncgl0253 | pcg3121 | FALSE | 3 |
| ncgl0253 | pcg3381 | FALSE | 3 |
| ncgl0254 | pcg0007_39 | TRUE | 3 |
| ncgl0280 | pcg1860 | TRUE | 3 |
| ncgl0281 | pcg0007_39 | TRUE | 3 |
| ncgl0281 | pcg3381 | FALSE | 3 |
| ncgl0304 | pcg0007_119 | FALSE | 4 |
| ncgl0304 | pcg0007_39 | TRUE | 4 |
| ncgl0304 | pcg0755 | FALSE | 4 |
| ncgl0304 | pcg1860 | FALSE | 4 |
| ncgl0304 | pcg2613 | FALSE | 4 |
| ncgl0304 | wt | FALSE | 4 |
| ncgl0306 | pcg0007_39 | TRUE | 4 |
| ncgl0306 | pcg0755 | FALSE | 4 |
| ncgl0306 | pcg2613 | FALSE | 4 |
| ncgl0306 | pcg3381 | FALSE | 4 |
| ncgl0355 | pcg0007_39 | TRUE | 2 |
| ncgl0355 | pcg0755 | FALSE | 2 |
| ncgl0355 | pcg1860 | FALSE | 2 |
| ncgl0355 | pcg2613 | FALSE | 2 |
| ncgl0355 | pcg3121 | FALSE | 2 |
| ncgl0355 | pcg3381 | FALSE | 2 |
| ncgl0355 | wt | FALSE | 2 |
| ncgl0359 | pcg0007_39 | FALSE | 2 |
| ncgl0359 | pcg1860 | TRUE | 2 |
| ncgl0378 | pcg0007_39 | TRUE | 3 |
| ncgl0378 | pcg3121 | FALSE | 3 |
| ncgl0378 | pcg3381 | FALSE | 3 |
| ncgl0380 | pcg0007_39 | TRUE | 3 |
| ncgl0386 | pcg0007_39 | TRUE | 3 |
| ncgl0386 | pcg1860 | TRUE | 3 |
| ncgl0411 | pcg0007_39 | TRUE | 3 |
| ncgl0411 | pcg1860 | TRUE | 3 |
| ncgl0411 | pcg3121 | FALSE | 3 |
| ncgl0419 | pcg0007_39 | TRUE | 3 |
| ncgl0419 | pcg1860 | TRUE | 3 |
| ncgl0419 | pcg3381 | FALSE | 3 |
| ncgl0439 | pcg0007_39 | FALSE | 3 |
| ncgl0439 | pcg1860 | TRUE | 3 |
| ncgl0439 | pcg3121 | FALSE | 3 |
| ncgl0439 | pcg3381 | FALSE | 3 |
| ncgl0445 | pcg0007_39 | TRUE | 3 |
| ncgl0445 | pcg3381 | FALSE | 3 |
| ncgl0453 | pcg0007_39 | TRUE | 3 |
| ncgl0453 | pcg3121 | FALSE | 3 |
| ncgl0453 | pcg3381 | FALSE | 3 |
| ncgl0456 | pcg3381 | TRUE | 4 |
| ncgl0458 | pcg0007_39 | TRUE | 3 |
| ncgl0458 | pcg0007_39 | TRUE | 3 |
| ncgl0458 | pcg0007_39 | FALSE | 3 |
| ncgl0458 | pcg3381 | FALSE | 3 |
| ncgl0458 | pcg3381 | FALSE | 3 |
| ncgl0465 | pcg0007_39 | TRUE | 3 |
| ncgl0465 | pcg3381 | FALSE | 3 |
| ncgl0471 | pcg0007_39 | FALSE | 3 |
| ncgl0471 | pcg0007_39 | TRUE | 3 |
| ncgl0471 | pcg0007_39 | FALSE | 3 |
| ncgl0471 | pcg1860 | FALSE | 3 |
| ncgl0471 | pcg1860 | FALSE | 3 |
| ncgl0471 | pcg1860 | FALSE | 3 |
| ncgl0471 | wt | FALSE | 3 |
| ncgl0472 | pcg0007_39 | TRUE | 3 |
| ncgl0482 | pcg0007_39 | TRUE | 3 |
| ncgl0482 | pcg3381 | FALSE | 3 |
| ncgl0509 | pcg0007_39 | TRUE | 3 |
| ncgl0509 | pcg1860 | TRUE | 3 |
| ncgl0509 | pcg3121 | FALSE | 3 |
| ncgl0509 | pcg3381 | FALSE | 3 |
| ncgl0527 | pcg0007_39 | TRUE | 3 |
| ncgl0527 | pcg1860 | TRUE | 3 |
| ncgl0527 | pcg3121 | FALSE | 3 |
| ncgl0527 | pcg3381 | FALSE | 3 |
| ncgl0531 | pcg0007_39 | TRUE | 3 |
| ncgl0531 | pcg0007_39 | TRUE | 3 |
| ncgl0531 | pcg1860 | TRUE | 3 |
| ncgl0531 | pcg3121 | FALSE | 3 |
| ncgl0531 | pcg3381 | FALSE | 3 |
| ncgl0531 | pcg3381 | FALSE | 3 |
| ncgl0546 | pcg0007_39 | TRUE | 4 |
| ncgl0548 | pcg0007_39 | TRUE | 3 |
| ncgl0548 | pcg1860 | TRUE | 3 |
| ncgl0548 | pcg3121 | FALSE | 3 |
| ncgl0548 | pcg3381 | FALSE | 3 |
| ncgl0565 | pcg0007_39 | FALSE | 3 |
| ncgl0565 | pcg1860 | TRUE | 3 |
| ncgl0565 | pcg3121 | FALSE | 3 |
| ncgl0565 | pcg3381 | FALSE | 3 |
| ncgl0578 | pcg0007_39 | TRUE | 4 |
| ncgl0578 | pcg3381 | FALSE | 4 |
| ncgl0581 | pcg0007_39 | TRUE | 3 |
| ncgl0581 | pcg0007_39 | TRUE | 3 |
| ncgl0581 | pcg3121 | FALSE | 3 |
| ncgl0581 | pcg3121 | FALSE | 3 |
| ncgl0581 | pcg3381 | FALSE | 3 |
| ncgl0581 | pcg3381 | FALSE | 3 |
| ncgl0598 | pcg0007_39 | TRUE | 4 |
| ncgl0598 | pcg3381 | FALSE | 4 |
| ncgl0601 | pcg0007_119 | FALSE | 3 |
| ncgl0601 | pcg0007_39 | TRUE | 3 |
| ncgl0601 | pcg3121 | FALSE | 3 |
| ncgl0601 | pcg3381 | FALSE | 3 |
| ncgl0601 | wt | FALSE | 3 |
| ncgl0631 | pcg0007_39 | TRUE | 2 |
| ncgl0631 | pcg1860 | FALSE | 2 |
| ncgl0631 | pcg3121 | FALSE | 2 |
| ncgl0634 | pcg0007 | TRUE | 1 |
| ncgl0634 | pcg0007 | TRUE | 2 |
| ncgl0634 | pcg0007_265 | FALSE | 1 |
| ncgl0634 | pcg0007_265 | FALSE | 2 |
| ncgl0634 | pcg0007_39 | FALSE | 1 |
| ncgl0634 | pcg0007_39 | FALSE | 2 |
| ncgl0634 | pcg0755 | FALSE | 1 |
| ncgl0634 | pcg0755 | FALSE | 2 |
| ncgl0634 | pcg1860 | TRUE | 1 |
| ncgl0634 | pcg1860 | TRUE | 2 |
| ncgl0634 | pcg3121 | FALSE | 1 |
| ncgl0634 | pcg3121 | FALSE | 2 |
| ncgl0634 | pcg3381 | FALSE | 1 |
| ncgl0634 | pcg3381 | FALSE | 2 |
| ncgl0634 | wt | FALSE | 1 |
| ncgl0634 | wt | FALSE | 2 |
| ncgl0634 | wt | FALSE | 1 |
| ncgl0634 | wt | FALSE | 2 |
| ncgl0634 | wt | FALSE | 1 |
| ncgl0634 | wt | FALSE | 2 |
| ncgl0634 | wt | FALSE | 1 |
| ncgl0634 | wt | FALSE | 2 |
| ncgl0634 | wt | FALSE | 1 |
| ncgl0634 | wt | FALSE | 2 |
| ncgl0636 | pcg0007_39 | TRUE | 3 |
| ncgl0636 | pcg3121 | FALSE | 3 |
| ncgl0636 | pcg3381 | FALSE | 3 |
| ncgl0637 | pcg0007_39 | TRUE | 3 |
| ncgl0637 | pcg1860 | TRUE | 3 |
| ncgl0637 | pcg3121 | FALSE | 3 |
| ncgl0637 | pcg3381 | FALSE | 3 |
| ncgl0638 | pcg0007_39 | TRUE | 3 |
| ncgl0638 | pcg0755 | FALSE | 3 |
| ncgl0640 | pcg1860 | TRUE | 3 |
| ncgl0640 | pcg3121 | FALSE | 3 |
| ncgl0640 | pcg3381 | FALSE | 3 |
| ncgl0645 | pcg0007_39 | TRUE | 3 |
| ncgl0645 | pcg3121 | FALSE | 3 |
| ncgl0645 | pcg3381 | FALSE | 3 |
| ncgl0646 | pcg0007_39 | TRUE | 3 |
| ncgl0646 | pcg3121 | FALSE | 3 |
| ncgl0646 | pcg3381 | FALSE | 3 |
| ncgl0655 | pcg0007_39 | TRUE | 3 |
| ncgl0655 | pcg1860 | TRUE | 3 |
| ncgl0655 | pcg1860 | TRUE | 3 |
| ncgl0655 | pcg3121 | FALSE | 3 |
| ncgl0655 | pcg3121 | FALSE | 3 |
| ncgl0655 | pcg3381 | FALSE | 3 |
| ncgl0655 | pcg3381 | FALSE | 3 |
| ncgl0668 | pcg0007_39 | TRUE | 3 |
| ncgl0668 | pcg0755 | FALSE | 3 |
| ncgl0668 | pcg2613 | FALSE | 3 |
| ncgl0668 | pcg3121 | FALSE | 3 |
| ncgl0668 | pcg3381 | FALSE | 3 |
| ncgl0679 | pcg0007_39 | TRUE | 3 |
| ncgl0679 | pcg1860 | FALSE | 3 |
| ncgl0679 | pcg3121 | FALSE | 3 |
| ncgl0679 | pcg3381 | FALSE | 3 |
| ncgl0689 | pcg0007_39 | FALSE | 3 |
| ncgl0689 | pcg0007_39 | TRUE | 3 |
| ncgl0689 | pcg3381 | FALSE | 3 |
| ncgl0694 | pcg0007_39 | FALSE | 3 |
| ncgl0694 | pcg1860 | TRUE | 3 |
| ncgl0694 | pcg3121 | FALSE | 3 |
| ncgl0694 | pcg3381 | FALSE | 3 |
| ncgl0697 | pcg0007_39 | FALSE | 3 |
| ncgl0697 | pcg0007_39 | TRUE | 3 |
| ncgl0697 | pcg3121 | FALSE | 3 |
| ncgl0697 | pcg3381 | FALSE | 3 |
| ncgl0698 | pcg0007_119 | FALSE | 3 |
| ncgl0698 | pcg0007_39 | TRUE | 3 |
| ncgl0698 | pcg0007_39 | TRUE | 3 |
| ncgl0698 | pcg1860 | FALSE | 3 |
| ncgl0698 | pcg3121 | FALSE | 3 |
| ncgl0698 | pcg3121 | FALSE | 3 |
| ncgl0708 | pcg0007_39 | TRUE | 3 |
| ncgl0743 | pcg0007_39 | FALSE | 4 |
| ncgl0743 | pcg3381 | TRUE | 4 |
| ncgl0767 | pcg0007_39 | TRUE | 4 |
| ncgl0767 | pcg2613 | FALSE | 4 |
| ncgl0768 | pcg0007_39 | FALSE | 3 |
| ncgl0768 | pcg0007_39 | TRUE | 3 |
| ncgl0768 | pcg3121 | FALSE | 3 |
| ncgl0768 | pcg3381 | FALSE | 3 |
| ncgl0780 | pcg0007 | TRUE | 1 |
| ncgl0780 | pcg0007_265 | TRUE | 1 |
| ncgl0780 | pcg0755 | TRUE | 1 |
| ncgl0780 | pcg3121 | TRUE | 1 |
| ncgl0780 | pcg3381 | TRUE | 1 |
| ncgl0817 | pcg0007 | TRUE | 1 |
| ncgl0817 | pcg0007_119 | FALSE | 1 |
| ncgl0817 | pcg0007_119 | TRUE | 1 |
| ncgl0817 | pcg0007_265 | TRUE | 1 |
| ncgl0817 | pcg0007_39 | TRUE | 1 |
| ncgl0817 | pcg0755 | FALSE | 1 |
| ncgl0817 | pcg0755 | TRUE | 1 |
| ncgl0817 | pcg1860 | TRUE | 1 |
| ncgl0817 | pcg2613 | FALSE | 1 |
| ncgl0817 | pcg3121 | TRUE | 1 |
| ncgl0817 | pcg3121 | TRUE | 1 |
| ncgl0817 | pcg3381 | TRUE | 1 |
| ncgl0817 | pcg3381 | TRUE | 1 |
| ncgl0823 | pcg0007_39 | FALSE | 3 |
| ncgl0823 | pcg0007_39 | TRUE | 3 |
| ncgl0823 | pcg3381 | FALSE | 3 |
| ncgl0827 | pcg0007_39 | TRUE | 4 |
| ncgl0827 | pcg3381 | FALSE | 4 |
| ncgl0847 | pcg0007_39 | TRUE | 2 |
| ncgl0847 | pcg0755 | FALSE | 2 |
| ncgl0847 | pcg2613 | FALSE | 2 |
| ncgl0847 | pcg3381 | FALSE | 2 |
| ncgl0860 | pcg0007_39 | TRUE | 4 |
| ncgl0861 | pcg0007_39 | TRUE | 3 |
| ncgl0861 | pcg1860 | TRUE | 3 |
| ncgl0861 | pcg3381 | FALSE | 3 |
| ncgl0865 | pcg0007_39 | FALSE | 1 |
| ncgl0865 | pcg0755 | FALSE | 1 |
| ncgl0865 | pcg3121 | TRUE | 1 |
| ncgl0865 | pcg3381 | FALSE | 1 |
| ncgl0877 | pcg0007_119 | FALSE | 4 |
| ncgl0877 | pcg0007_39 | TRUE | 4 |
| ncgl0877 | pcg3381 | FALSE | 4 |
| ncgl0877 | wt | FALSE | 4 |
| ncgl0893 | pcg0007_39 | TRUE | 3 |
| ncgl0893 | pcg3121 | FALSE | 3 |
| ncgl0893 | pcg3381 | FALSE | 3 |
| ncgl0897 | pcg0007_39 | TRUE | 3 |
| ncgl0897 | pcg3381 | FALSE | 3 |
| ncgl0901 | pcg0007_39 | TRUE | 3 |
| ncgl0901 | pcg1860 | TRUE | 3 |
| ncgl0901 | pcg3121 | FALSE | 3 |
| ncgl0901 | pcg3381 | FALSE | 3 |
| ncgl0909 | pcg0007_39 | FALSE | 3 |
| ncgl0909 | pcg3121 | TRUE | 3 |
| ncgl0909 | pcg3381 | FALSE | 3 |
| ncgl0965 | pcg1860 | TRUE | 3 |
| ncgl0966 | pcg1860 | FALSE | 4 |
| ncgl0966 | pcg3121 | TRUE | 4 |
| ncgl0966 | wt | FALSE | 4 |
| ncgl0976 | pcg0007 | FALSE | 1 |
| ncgl0976 | pcg0007_119 | FALSE | 1 |
| ncgl0976 | pcg0007_39 | TRUE | 1 |
| ncgl0976 | pcg0755 | FALSE | 1 |
| ncgl0976 | pcg1860 | FALSE | 1 |
| ncgl0976 | pcg3121 | FALSE | 1 |
| ncgl0976 | pcg3381 | FALSE | 1 |
| ncgl1016 | pcg0007_39 | TRUE | 3 |
| ncgl1016 | pcg1860 | TRUE | 3 |
| ncgl1016 | pcg3121 | FALSE | 3 |
| ncgl1016 | pcg3381 | FALSE | 3 |
| ncgl1025 | pcg0007_39 | TRUE | 3 |
| ncgl1025 | pcg1860 | TRUE | 3 |
| ncgl1025 | pcg3121 | FALSE | 3 |
| ncgl1044 | pcg0007_39 | TRUE | 4 |
| ncgl1049 | pcg0007_39 | TRUE | 3 |
| ncgl1049 | pcg3381 | FALSE | 3 |
| ncgl1062 | pcg0007_39 | TRUE | 3 |
| ncgl1062 | pcg2613 | FALSE | 3 |
| ncgl1064 | pcg0007 | FALSE | 1 |
| ncgl1064 | pcg0007_119 | FALSE | 1 |
| ncgl1064 | pcg0007_39 | FALSE | 1 |
| ncgl1064 | pcg0755 | TRUE | 1 |
| ncgl1064 | pcg1860 | FALSE | 1 |
| ncgl1064 | pcg2613 | FALSE | 1 |
| ncgl1064 | pcg3381 | FALSE | 1 |
| ncgl1064 | wt | FALSE | 1 |
| ncgl1065 | pcg0007_39 | TRUE | 4 |
| ncgl1080 | pcg0007_39 | TRUE | 3 |
| ncgl1080 | pcg3121 | FALSE | 3 |
| ncgl1080 | pcg3381 | FALSE | 3 |
| ncgl1084 | pcg0007_119 | FALSE | 2 |
| ncgl1084 | pcg0007_39 | TRUE | 2 |
| ncgl1084 | wt | FALSE | 2 |
| ncgl1129 | pcg0007_39 | TRUE | 3 |
| ncgl1129 | pcg3121 | FALSE | 3 |
| ncgl1129 | pcg3381 | FALSE | 3 |
| ncgl1133 | pcg0007 | TRUE | 1 |
| ncgl1133 | pcg0007_119 | FALSE | 1 |
| ncgl1133 | pcg0007_265 | TRUE | 1 |
| ncgl1133 | pcg0007_39 | TRUE | 1 |
| ncgl1133 | pcg0755 | FALSE | 1 |
| ncgl1133 | pcg3121 | FALSE | 1 |
| ncgl1133 | pcg3381 | TRUE | 1 |
| ncgl1136 | pcg0007 | FALSE | 1 |
| ncgl1136 | pcg0007_119 | FALSE | 1 |
| ncgl1136 | pcg0007_39 | TRUE | 1 |
| ncgl1136 | pcg0755 | TRUE | 1 |
| ncgl1136 | pcg1860 | TRUE | 1 |
| ncgl1136 | pcg2613 | FALSE | 1 |
| ncgl1136 | pcg3121 | TRUE | 1 |
| ncgl1136 | pcg3381 | TRUE | 1 |
| ncgl1136 | wt | FALSE | 1 |
| ncgl1140 | pcg0007_39 | TRUE | 2 |
| ncgl1140 | pcg0755 | FALSE | 2 |
| ncgl1140 | pcg2613 | FALSE | 2 |
| ncgl1140 | pcg3121 | FALSE | 2 |
| ncgl1140 | wt | FALSE | 2 |
| ncgl1143 | pcg0007_39 | TRUE | 2 |
| ncgl1143 | pcg0007_39 | TRUE | 3 |
| ncgl1143 | pcg3121 | FALSE | 2 |
| ncgl1143 | pcg3121 | FALSE | 3 |
| ncgl1143 | pcg3381 | FALSE | 2 |
| ncgl1143 | pcg3381 | FALSE | 3 |
| ncgl1147 | pcg0007_39 | TRUE | 3 |
| ncgl1147 | pcg3121 | FALSE | 3 |
| ncgl1147 | pcg3381 | FALSE | 3 |
| ncgl1152 | pcg0007_39 | TRUE | 2 |
| ncgl1152 | pcg0007_39 | TRUE | 3 |
| ncgl1152 | pcg0755 | FALSE | 2 |
| ncgl1152 | pcg0755 | FALSE | 3 |
| ncgl1152 | pcg1860 | FALSE | 2 |
| ncgl1152 | pcg1860 | FALSE | 3 |
| ncgl1152 | pcg3381 | FALSE | 2 |
| ncgl1152 | pcg3381 | FALSE | 3 |
| ncgl1152 | wt | FALSE | 2 |
| ncgl1152 | wt | FALSE | 3 |
| ncgl1175 | pcg0007_39 | TRUE | 3 |
| ncgl1175 | pcg1860 | TRUE | 3 |
| ncgl1175 | pcg3381 | FALSE | 3 |
| ncgl1179 | pcg0007_39 | TRUE | 3 |
| ncgl1179 | pcg0755 | FALSE | 3 |
| ncgl1179 | pcg1860 | FALSE | 3 |
| ncgl1179 | pcg2613 | FALSE | 3 |
| ncgl1179 | pcg3121 | FALSE | 3 |
| ncgl1179 | pcg3381 | FALSE | 3 |
| ncgl1179 | wt | FALSE | 3 |
| ncgl1181 | pcg0007_39 | TRUE | 3 |
| ncgl1181 | pcg3121 | FALSE | 3 |
| ncgl1181 | pcg3381 | FALSE | 3 |
| ncgl1202 | pcg0007 | FALSE | 4 |
| ncgl1202 | pcg0007_265 | FALSE | 4 |
| ncgl1202 | pcg0007_39 | FALSE | 4 |
| ncgl1202 | pcg1860 | FALSE | 4 |
| ncgl1202 | pcg3381 | TRUE | 4 |
| ncgl1203 | pcg0007_39 | TRUE | 3 |
| ncgl1203 | pcg3381 | FALSE | 3 |
| ncgl1208 | pcg0007_39 | TRUE | 4 |
| ncgl1209 | pcg0007_39 | TRUE | 3 |
| ncgl1209 | pcg1860 | TRUE | 3 |
| ncgl1209 | pcg3121 | FALSE | 3 |
| ncgl1209 | pcg3121 | FALSE | 3 |
| ncgl1209 | pcg3381 | FALSE | 3 |
| ncgl1209 | pcg3381 | FALSE | 3 |
| ncgl1214 | pcg0007 | TRUE | 1 |
| ncgl1214 | pcg0007_119 | TRUE | 1 |
| ncgl1214 | pcg0007_265 | FALSE | 1 |
| ncgl1214 | pcg0007_39 | TRUE | 1 |
| ncgl1214 | pcg0755 | FALSE | 1 |
| ncgl1214 | pcg1860 | FALSE | 1 |
| ncgl1214 | pcg3121 | FALSE | 1 |
| ncgl1214 | pcg3381 | FALSE | 1 |
| ncgl1224 | pcg0007_39 | TRUE | 4 |
| ncgl1241 | pcg0007 | TRUE | 1 |
| ncgl1241 | pcg0007_119 | FALSE | 1 |
| ncgl1241 | pcg0007_39 | TRUE | 1 |
| ncgl1241 | pcg0755 | FALSE | 1 |
| ncgl1241 | pcg1860 | FALSE | 1 |
| ncgl1241 | pcg3121 | FALSE | 1 |
| ncgl1241 | pcg3381 | FALSE | 1 |
| ncgl1261 | pcg0007_119 | FALSE | 3 |
| ncgl1261 | pcg0007_39 | TRUE | 3 |
| ncgl1261 | pcg0755 | FALSE | 3 |
| ncgl1261 | pcg2613 | FALSE | 3 |
| ncgl1261 | pcg3381 | FALSE | 3 |
| ncgl1261 | wt | FALSE | 3 |
| ncgl1262 | pcg0007_39 | TRUE | 4 |
| ncgl1262 | pcg0755 | TRUE | 4 |
| ncgl1262 | pcg1860 | TRUE | 4 |
| ncgl1262 | pcg2613 | TRUE | 4 |
| ncgl1262 | pcg3121 | FALSE | 4 |
| ncgl1262 | pcg3381 | FALSE | 4 |
| ncgl1262 | wt | FALSE | 4 |
| ncgl1263 | pcg0007_39 | TRUE | 4 |
| ncgl1263 | pcg3381 | FALSE | 4 |
| ncgl1267 | pcg0007_39 | TRUE | 3 |
| ncgl1267 | pcg0755 | FALSE | 3 |
| ncgl1267 | pcg1860 | FALSE | 3 |
| ncgl1267 | pcg2613 | FALSE | 3 |
| ncgl1267 | pcg3381 | FALSE | 3 |
| ncgl1267 | wt | FALSE | 3 |
| ncgl1267 | wt | FALSE | 3 |
| ncgl1267 | wt | FALSE | 3 |
| ncgl1267 | wt | FALSE | 3 |
| ncgl1267 | wt | FALSE | 3 |
| ncgl1267 | wt | FALSE | 3 |
| ncgl1267 | wt | FALSE | 3 |
| ncgl1277 | pcg0007_39 | TRUE | 3 |
| ncgl1277 | pcg0755 | FALSE | 3 |
| ncgl1277 | pcg2613 | FALSE | 3 |
| ncgl1277 | pcg3121 | FALSE | 3 |
| ncgl1277 | pcg3381 | FALSE | 3 |
| ncgl1277 | wt | FALSE | 3 |
| ncgl1301 | pcg0007_119 | FALSE | 3 |
| ncgl1301 | pcg0007_39 | TRUE | 3 |
| ncgl1301 | pcg1860 | FALSE | 3 |
| ncgl1301 | pcg2613 | FALSE | 3 |
| ncgl1301 | pcg3121 | FALSE | 3 |
| ncgl1301 | pcg3381 | FALSE | 3 |
| ncgl1305 | pcg0007 | FALSE | 1 |
| ncgl1305 | pcg0007 | FALSE | 3 |
| ncgl1305 | pcg0007 | FALSE | 1 |
| ncgl1305 | pcg0007 | FALSE | 3 |
| ncgl1305 | pcg0007_265 | TRUE | 1 |
| ncgl1305 | pcg0007_265 | TRUE | 3 |
| ncgl1305 | pcg0007_39 | FALSE | 1 |
| ncgl1305 | pcg0007_39 | FALSE | 3 |
| ncgl1305 | pcg0007_39 | TRUE | 1 |
| ncgl1305 | pcg0007_39 | TRUE | 3 |
| ncgl1305 | pcg0755 | TRUE | 1 |
| ncgl1305 | pcg0755 | TRUE | 3 |
| ncgl1305 | pcg0755 | FALSE | 1 |
| ncgl1305 | pcg0755 | FALSE | 3 |
| ncgl1305 | pcg1860 | TRUE | 1 |
| ncgl1305 | pcg1860 | TRUE | 3 |
| ncgl1305 | pcg1860 | TRUE | 1 |
| ncgl1305 | pcg1860 | TRUE | 3 |
| ncgl1305 | pcg3121 | FALSE | 1 |
| ncgl1305 | pcg3121 | FALSE | 3 |
| ncgl1305 | pcg3121 | TRUE | 1 |
| ncgl1305 | pcg3121 | TRUE | 3 |
| ncgl1305 | pcg3381 | FALSE | 1 |
| ncgl1305 | pcg3381 | FALSE | 3 |
| ncgl1305 | pcg3381 | FALSE | 1 |
| ncgl1305 | pcg3381 | FALSE | 3 |
| ncgl1322 | pcg0007_39 | TRUE | 4 |
| ncgl1322 | pcg0755 | FALSE | 4 |
| ncgl1322 | pcg1860 | FALSE | 4 |
| ncgl1322 | pcg2613 | FALSE | 4 |
| ncgl1322 | pcg3121 | FALSE | 4 |
| ncgl1322 | pcg3381 | FALSE | 4 |
| ncgl1322 | wt | FALSE | 4 |
| ncgl1330 | pcg0007_39 | TRUE | 3 |
| ncgl1330 | pcg3381 | FALSE | 3 |
| ncgl1332 | pcg0007_119 | FALSE | 3 |
| ncgl1332 | pcg0007_39 | TRUE | 3 |
| ncgl1332 | pcg0755 | FALSE | 3 |
| ncgl1332 | pcg1860 | TRUE | 3 |
| ncgl1332 | pcg3121 | FALSE | 3 |
| ncgl1332 | pcg3381 | FALSE | 3 |
| ncgl1332 | wt | FALSE | 3 |
| ncgl1344 | pcg0007_39 | TRUE | 4 |
| ncgl1344 | pcg3381 | FALSE | 4 |
| ncgl1347 | pcg0007_39 | TRUE | 4 |
| ncgl1362 | pcg0007_39 | FALSE | 3 |
| ncgl1362 | pcg1860 | TRUE | 3 |
| ncgl1362 | pcg3121 | FALSE | 3 |
| ncgl1362 | pcg3381 | FALSE | 3 |
| ncgl1363 | pcg0007_39 | TRUE | 3 |
| ncgl1363 | pcg3121 | FALSE | 3 |
| ncgl1364 | pcg0007_119 | FALSE | 3 |
| ncgl1364 | pcg0007_39 | TRUE | 3 |
| ncgl1364 | pcg0755 | FALSE | 3 |
| ncgl1364 | pcg1860 | TRUE | 3 |
| ncgl1364 | pcg2613 | FALSE | 3 |
| ncgl1364 | pcg3121 | FALSE | 3 |
| ncgl1364 | pcg3381 | FALSE | 3 |
| ncgl1364 | wt | FALSE | 3 |
| ncgl1366 | pcg0007_39 | FALSE | 3 |
| ncgl1366 | pcg0007_39 | TRUE | 3 |
| ncgl1366 | pcg0007_39 | TRUE | 3 |
| ncgl1366 | pcg1860 | TRUE | 3 |
| ncgl1366 | pcg3121 | FALSE | 3 |
| ncgl1366 | pcg3121 | FALSE | 3 |
| ncgl1366 | pcg3381 | FALSE | 3 |
| ncgl1366 | pcg3381 | FALSE | 3 |
| ncgl1367 | pcg0007_39 | TRUE | 3 |
| ncgl1367 | pcg3121 | FALSE | 3 |
| ncgl1367 | pcg3381 | FALSE | 3 |
| ncgl1370 | pcg0007_39 | FALSE | 3 |
| ncgl1370 | pcg1860 | TRUE | 3 |
| ncgl1370 | pcg3121 | FALSE | 3 |
| ncgl1370 | pcg3381 | FALSE | 3 |
| ncgl1371 | pcg0007_119 | FALSE | 3 |
| ncgl1371 | pcg0007_39 | TRUE | 3 |
| ncgl1371 | pcg0755 | FALSE | 3 |
| ncgl1371 | pcg1860 | FALSE | 3 |
| ncgl1371 | pcg2613 | FALSE | 3 |
| ncgl1371 | wt | FALSE | 3 |
| ncgl1372 | pcg0007_39 | TRUE | 3 |
| ncgl1372 | pcg1860 | TRUE | 3 |
| ncgl1372 | pcg3121 | FALSE | 3 |
| ncgl1372 | pcg3381 | FALSE | 3 |
| ncgl1378 | pcg0007_39 | TRUE | 3 |
| ncgl1378 | pcg3121 | FALSE | 3 |
| ncgl1399 | pcg0007_39 | TRUE | 3 |
| ncgl1399 | pcg3121 | FALSE | 3 |
| ncgl1399 | pcg3381 | FALSE | 3 |
| ncgl1402 | pcg0007_39 | FALSE | 3 |
| ncgl1402 | pcg1860 | TRUE | 3 |
| ncgl1454 | pcg0007_39 | TRUE | 3 |
| ncgl1454 | pcg3121 | FALSE | 3 |
| ncgl1454 | pcg3381 | FALSE | 3 |
| ncgl1455 | pcg0007_39 | TRUE | 3 |
| ncgl1455 | pcg3121 | FALSE | 3 |
| ncgl1455 | pcg3381 | FALSE | 3 |
| ncgl1484 | pcg0007_119 | FALSE | 4 |
| ncgl1484 | pcg0007_39 | TRUE | 4 |
| ncgl1484 | pcg0755 | FALSE | 4 |
| ncgl1484 | pcg2613 | FALSE | 4 |
| ncgl1484 | pcg3121 | FALSE | 4 |
| ncgl1484 | wt | FALSE | 4 |
| ncgl1506 | pcg0007_39 | TRUE | 3 |
| ncgl1506 | pcg1860 | TRUE | 3 |
| ncgl1506 | pcg3121 | FALSE | 3 |
| ncgl1506 | pcg3381 | FALSE | 3 |
| ncgl1507 | pcg1860 | TRUE | 3 |
| ncgl1507 | pcg3121 | FALSE | 3 |
| ncgl1507 | pcg3381 | FALSE | 3 |
| ncgl1511 | pcg0007_119 | FALSE | 4 |
| ncgl1511 | pcg0007_39 | TRUE | 4 |
| ncgl1511 | pcg0755 | FALSE | 4 |
| ncgl1511 | pcg2613 | FALSE | 4 |
| ncgl1511 | pcg3121 | FALSE | 4 |
| ncgl1511 | pcg3381 | FALSE | 4 |
| ncgl1511 | wt | FALSE | 4 |
| ncgl1512 | pcg0007 | FALSE | 1 |
| ncgl1512 | pcg0007_39 | FALSE | 1 |
| ncgl1512 | pcg0007_39 | TRUE | 1 |
| ncgl1512 | pcg0755 | FALSE | 1 |
| ncgl1512 | pcg0755 | FALSE | 1 |
| ncgl1512 | pcg1860 | FALSE | 1 |
| ncgl1512 | pcg1860 | TRUE | 1 |
| ncgl1512 | pcg3381 | FALSE | 1 |
| ncgl1512 | pcg3381 | TRUE | 1 |
| ncgl1514 | pcg0007 | TRUE | 1 |
| ncgl1514 | pcg0007_119 | TRUE | 1 |
| ncgl1514 | pcg0007_265 | FALSE | 1 |
| ncgl1514 | pcg0007_39 | TRUE | 1 |
| ncgl1514 | pcg0755 | FALSE | 1 |
| ncgl1514 | pcg1860 | FALSE | 1 |
| ncgl1514 | pcg3121 | FALSE | 1 |
| ncgl1514 | pcg3381 | TRUE | 1 |
| ncgl1521 | pcg0007_39 | TRUE | 2 |
| ncgl1521 | pcg0007_39 | TRUE | 3 |
| ncgl1521 | pcg1860 | FALSE | 2 |
| ncgl1521 | pcg1860 | FALSE | 3 |
| ncgl1521 | pcg3121 | FALSE | 2 |
| ncgl1521 | pcg3121 | FALSE | 3 |
| ncgl1523 | pcg0007 | FALSE | 1 |
| ncgl1523 | pcg0007_119 | FALSE | 1 |
| ncgl1523 | pcg0007_39 | TRUE | 1 |
| ncgl1523 | pcg0755 | TRUE | 1 |
| ncgl1523 | pcg1860 | FALSE | 1 |
| ncgl1523 | pcg3121 | TRUE | 1 |
| ncgl1525 | pcg0007_39 | FALSE | 4 |
| ncgl1525 | pcg3381 | TRUE | 4 |
| ncgl1545 | pcg0007_39 | TRUE | 4 |
| ncgl1545 | wt | FALSE | 4 |
| ncgl1590 | pcg3381 | FALSE | 4 |
| ncgl1590 | wt | FALSE | 4 |
| ncgl1590 | wt | FALSE | 4 |
| ncgl1590 | wt | FALSE | 4 |
| ncgl1590 | wt | TRUE | 4 |
| ncgl1592 | pcg0007_39 | TRUE | 3 |
| ncgl1592 | pcg3121 | FALSE | 3 |
| ncgl1592 | pcg3381 | FALSE | 3 |
| ncgl1607 | pcg0007_39 | TRUE | 4 |
| ncgl1607 | pcg0755 | FALSE | 4 |
| ncgl1607 | pcg2613 | FALSE | 4 |
| ncgl1607 | wt | FALSE | 4 |
| ncgl1817 | pcg0007_39 | TRUE | 3 |
| ncgl1817 | pcg1860 | TRUE | 3 |
| ncgl1817 | pcg3121 | FALSE | 3 |
| ncgl1817 | pcg3381 | FALSE | 3 |
| ncgl1844 | pcg0007_39 | TRUE | 3 |
| ncgl1844 | pcg1860 | TRUE | 3 |
| ncgl1868 | pcg0007 | TRUE | 1 |
| ncgl1868 | pcg0007_119 | TRUE | 1 |
| ncgl1868 | pcg0007_39 | TRUE | 1 |
| ncgl1868 | pcg0755 | TRUE | 1 |
| ncgl1868 | pcg1860 | TRUE | 1 |
| ncgl1868 | pcg3121 | FALSE | 1 |
| ncgl1868 | pcg3381 | FALSE | 1 |
| ncgl1875 | pcg0007_39 | TRUE | 3 |
| ncgl1875 | pcg1860 | TRUE | 3 |
| ncgl1875 | pcg3121 | FALSE | 3 |
| ncgl1875 | pcg3381 | FALSE | 3 |
| ncgl1875 | pcg3381 | FALSE | 3 |
| ncgl1877 | pcg0007_39 | TRUE | 3 |
| ncgl1877 | pcg3121 | FALSE | 3 |
| ncgl1885 | pcg0007_39 | TRUE | 3 |
| ncgl1885 | pcg1860 | TRUE | 3 |
| ncgl1896 | pcg0007 | FALSE | 1 |
| ncgl1896 | pcg0007 | FALSE | 1 |
| ncgl1896 | pcg0007_265 | FALSE | 1 |
| ncgl1896 | pcg0007_265 | FALSE | 1 |
| ncgl1896 | pcg0007_39 | TRUE | 1 |
| ncgl1896 | pcg0007_39 | FALSE | 1 |
| ncgl1896 | pcg0755 | FALSE | 1 |
| ncgl1896 | pcg1860 | FALSE | 1 |
| ncgl1896 | pcg3121 | FALSE | 1 |
| ncgl1896 | pcg3381 | FALSE | 1 |
| ncgl1896 | pcg3381 | FALSE | 1 |
| ncgl1896 | wt | FALSE | 1 |
| ncgl1898 | pcg0007_265 | TRUE | 1 |
| ncgl1898 | pcg0755 | FALSE | 1 |
| ncgl1898 | pcg1860 | FALSE | 1 |
| ncgl1899 | pcg0007_39 | TRUE | 3 |
| ncgl1899 | pcg3381 | FALSE | 3 |
| ncgl1911 | pcg0007_119 | FALSE | 3 |
| ncgl1911 | pcg0007_39 | TRUE | 3 |
| ncgl1911 | pcg0755 | FALSE | 3 |
| ncgl1911 | pcg1860 | TRUE | 3 |
| ncgl1911 | pcg2613 | FALSE | 3 |
| ncgl1911 | pcg3121 | FALSE | 3 |
| ncgl1911 | pcg3121 | FALSE | 3 |
| ncgl1911 | pcg3381 | FALSE | 3 |
| ncgl1911 | pcg3381 | FALSE | 3 |
| ncgl1912 | pcg0007_39 | TRUE | 3 |
| ncgl1913 | pcg0007_39 | TRUE | 3 |
| ncgl1913 | pcg3121 | FALSE | 3 |
| ncgl1913 | pcg3381 | FALSE | 3 |
| ncgl1915 | pcg0007_39 | FALSE | 3 |
| ncgl1915 | pcg0007_39 | TRUE | 3 |
| ncgl1915 | pcg3121 | FALSE | 3 |
| ncgl1917 | pcg0007_39 | TRUE | 3 |
| ncgl1917 | pcg3381 | FALSE | 3 |
| ncgl1918 | pcg1860 | TRUE | 3 |
| ncgl1918 | pcg3121 | FALSE | 3 |
| ncgl1926 | pcg0007_39 | FALSE | 2 |
| ncgl1926 | pcg1860 | FALSE | 2 |
| ncgl1926 | pcg3121 | FALSE | 2 |
| ncgl1926 | pcg3381 | TRUE | 2 |
| ncgl1948 | pcg0007_39 | TRUE | 4 |
| ncgl1948 | wt | FALSE | 4 |
| ncgl1978 | pcg0007_39 | TRUE | 3 |
| ncgl1978 | pcg3121 | FALSE | 3 |
| ncgl1997 | pcg0007_119 | FALSE | 3 |
| ncgl1997 | pcg0007_39 | TRUE | 3 |
| ncgl1997 | pcg0755 | FALSE | 3 |
| ncgl1997 | pcg1860 | FALSE | 3 |
| ncgl1997 | pcg2613 | TRUE | 3 |
| ncgl1997 | pcg3121 | FALSE | 3 |
| ncgl1997 | pcg3381 | FALSE | 3 |
| ncgl1997 | wt | FALSE | 3 |
| ncgl2017 | pcg0007_39 | TRUE | 3 |
| ncgl2017 | pcg3381 | FALSE | 3 |
| ncgl2025 | pcg0007_39 | TRUE | 3 |
| ncgl2025 | pcg1860 | TRUE | 3 |
| ncgl2031 | pcg0007_39 | TRUE | 3 |
| ncgl2031 | pcg3121 | FALSE | 3 |
| ncgl2031 | pcg3381 | FALSE | 3 |
| ncgl2032 | pcg0007_39 | TRUE | 3 |
| ncgl2032 | pcg3121 | FALSE | 3 |
| ncgl2032 | pcg3381 | FALSE | 3 |
| ncgl2060 | pcg0007_39 | TRUE | 3 |
| ncgl2060 | pcg1860 | TRUE | 3 |
| ncgl2060 | pcg3121 | FALSE | 3 |
| ncgl2066 | pcg0007_39 | TRUE | 3 |
| ncgl2066 | pcg1860 | TRUE | 3 |
| ncgl2066 | pcg3121 | FALSE | 3 |
| ncgl2066 | pcg3381 | FALSE | 3 |
| ncgl2081 | pcg0007_39 | TRUE | 3 |
| ncgl2082 | pcg0007_39 | TRUE | 3 |
| ncgl2082 | pcg3381 | FALSE | 3 |
| ncgl2083 | pcg0007_39 | TRUE | 3 |
| ncgl2083 | pcg0755 | FALSE | 3 |
| ncgl2083 | wt | FALSE | 3 |
| ncgl2084 | pcg0007_39 | TRUE | 3 |
| ncgl2084 | pcg3381 | FALSE | 3 |
| ncgl2091 | pcg0007_39 | TRUE | 4 |
| ncgl2091 | pcg3381 | FALSE | 4 |
| ncgl2104 | pcg0007_39 | TRUE | 3 |
| ncgl2104 | pcg0007_39 | TRUE | 3 |
| ncgl2104 | pcg3121 | FALSE | 3 |
| ncgl2104 | pcg3121 | FALSE | 3 |
| ncgl2104 | pcg3381 | FALSE | 3 |
| ncgl2104 | wt | FALSE | 3 |
| ncgl2126 | pcg0007 | FALSE | 2 |
| ncgl2126 | pcg0007_39 | FALSE | 2 |
| ncgl2126 | pcg1860 | TRUE | 2 |
| ncgl2126 | pcg3121 | FALSE | 2 |
| ncgl2133 | pcg0007_39 | TRUE | 2 |
| ncgl2133 | pcg0007_39 | TRUE | 3 |
| ncgl2133 | pcg1860 | FALSE | 2 |
| ncgl2133 | pcg1860 | FALSE | 3 |
| ncgl2133 | wt | FALSE | 2 |
| ncgl2133 | wt | FALSE | 3 |
| ncgl2163 | pcg0007_39 | TRUE | 3 |
| ncgl2163 | pcg1860 | TRUE | 3 |
| ncgl2167 | pcg0007_39 | FALSE | 2 |
| ncgl2167 | pcg0007_39 | FALSE | 2 |
| ncgl2167 | pcg0755 | FALSE | 2 |
| ncgl2167 | pcg1860 | FALSE | 2 |
| ncgl2167 | pcg1860 | TRUE | 2 |
| ncgl2167 | pcg2613 | FALSE | 2 |
| ncgl2167 | pcg3121 | FALSE | 2 |
| ncgl2167 | pcg3381 | FALSE | 2 |
| ncgl2167 | pcg3381 | TRUE | 2 |
| ncgl2168 | pcg0007_39 | TRUE | 3 |
| ncgl2169 | pcg0007_39 | TRUE | 3 |
| ncgl2169 | pcg0755_promoter | FALSE | 3 |
| ncgl2169 | pcg1860 | TRUE | 3 |
| ncgl2169 | pcg3121 | FALSE | 3 |
| ncgl2169 | pcg3381 | FALSE | 3 |
| ncgl2211 | pcg0007_39 | TRUE | 3 |
| ncgl2211 | pcg3381 | FALSE | 3 |
| ncgl2230 | pcg0007_39 | FALSE | 3 |
| ncgl2230 | pcg0007_39 | TRUE | 3 |
| ncgl2230 | pcg0007_39 | TRUE | 3 |
| ncgl2230 | pcg3121 | FALSE | 3 |
| ncgl2230 | pcg3381 | FALSE | 3 |
| ncgl2239 | pcg0007_39 | TRUE | 3 |
| ncgl2239 | pcg3121 | FALSE | 3 |
| ncgl2239 | pcg3381 | FALSE | 3 |
| ncgl2240 | pcg0007_119 | FALSE | 3 |
| ncgl2240 | pcg0007_39 | TRUE | 3 |
| ncgl2240 | pcg0755 | FALSE | 3 |
| ncgl2240 | pcg1860 | TRUE | 3 |
| ncgl2240 | pcg2613 | FALSE | 3 |
| ncgl2240 | pcg3121 | FALSE | 3 |
| ncgl2240 | pcg3381 | FALSE | 3 |
| ncgl2240 | wt | FALSE | 3 |
| ncgl2241 | pcg0007_39 | FALSE | 3 |
| ncgl2241 | pcg1860 | TRUE | 3 |
| ncgl2241 | pcg3121 | FALSE | 3 |
| ncgl2241 | pcg3381 | FALSE | 3 |
| ncgl2245 | pcg0007_39 | TRUE | 3 |
| ncgl2245 | pcg3121 | FALSE | 3 |
| ncgl2245 | pcg3381 | FALSE | 3 |
| ncgl2250 | pcg0007_119 | FALSE | 4 |
| ncgl2250 | pcg0007_39 | TRUE | 4 |
| ncgl2250 | pcg0755 | FALSE | 4 |
| ncgl2250 | pcg2613 | FALSE | 4 |
| ncgl2250 | pcg3121 | FALSE | 4 |
| ncgl2250 | pcg3381 | FALSE | 4 |
| ncgl2257 | pcg0007_39 | TRUE | 3 |
| ncgl2257 | pcg3121 | FALSE | 3 |
| ncgl2257 | pcg3381 | FALSE | 3 |
| ncgl2298 | pcg0007_39 | TRUE | 3 |
| ncgl2298 | pcg0007_39 | TRUE | 3 |
| ncgl2298 | pcg1860 | FALSE | 3 |
| ncgl2298 | pcg3381 | FALSE | 3 |
| ncgl2327 | pcg0007_39 | TRUE | 4 |
| ncgl2327 | pcg2613 | FALSE | 4 |
| ncgl2327 | pcg3381 | FALSE | 4 |
| ncgl2327 | wt | FALSE | 4 |
| ncgl2350 | pcg0007_39 | TRUE | 3 |
| ncgl2350 | pcg3121 | FALSE | 3 |
| ncgl2350 | pcg3381 | FALSE | 3 |
| ncgl2373 | pcg0007_39 | TRUE | 3 |
| ncgl2374 | pcg0007_39 | TRUE | 3 |
| ncgl2377 | pcg0007_39 | TRUE | 3 |
| ncgl2377 | pcg3121 | FALSE | 3 |
| ncgl2385 | pcg0007_39 | TRUE | 3 |
| ncgl2385 | pcg3381 | FALSE | 3 |
| ncgl2405 | pcg0007_39 | FALSE | 3 |
| ncgl2405 | pcg1860 | TRUE | 3 |
| ncgl2406 | pcg0007_39 | TRUE | 3 |
| ncgl2406 | pcg3121 | FALSE | 3 |
| ncgl2406 | pcg3381 | FALSE | 3 |
| ncgl2425 | pcg0007_119 | FALSE | 3 |
| ncgl2425 | pcg0007_39 | TRUE | 3 |
| ncgl2425 | pcg3121 | FALSE | 3 |
| ncgl2425 | pcg3381 | FALSE | 3 |
| ncgl2425 | wt | FALSE | 3 |
| ncgl2440 | pcg0007_39 | FALSE | 3 |
| ncgl2440 | pcg1860 | FALSE | 3 |
| ncgl2440 | pcg3121 | TRUE | 3 |
| ncgl2440 | pcg3381 | FALSE | 3 |
| ncgl2449 | pcg0007_39 | TRUE | 4 |
| ncgl2449 | wt | FALSE | 4 |
| ncgl2465 | pcg0007_39 | FALSE | 3 |
| ncgl2465 | pcg0007_39 | TRUE | 3 |
| ncgl2465 | pcg3381 | FALSE | 3 |
| ncgl2481 | pcg0007_39 | TRUE | 4 |
| ncgl2481 | wt | FALSE | 4 |
| ncgl2482 | pcg0007_39 | TRUE | 3 |
| ncgl2482 | pcg0007_39 | FALSE | 3 |
| ncgl2482 | pcg0755 | FALSE | 3 |
| ncgl2482 | pcg2613 | FALSE | 3 |
| ncgl2482 | pcg3121 | FALSE | 3 |
| ncgl2482 | pcg3381 | TRUE | 3 |
| ncgl2482 | pcg3381 | FALSE | 3 |
| ncgl2482 | wt | FALSE | 3 |
| ncgl2483 | pcg0007_39 | TRUE | 3 |
| ncgl2483 | pcg1860 | TRUE | 3 |
| ncgl2483 | pcg3381 | FALSE | 3 |
| ncgl2485 | pcg0007_39 | TRUE | 3 |
| ncgl2485 | pcg3381 | FALSE | 3 |
| ncgl2491 | pcg0007_119 | FALSE | 4 |
| ncgl2491 | pcg0007_39 | TRUE | 4 |
| ncgl2491 | pcg2613 | FALSE | 4 |
| ncgl2491 | pcg3381 | FALSE | 4 |
| ncgl2491 | wt | FALSE | 4 |
| ncgl2499 | pcg0007_39 | TRUE | 4 |
| ncgl2500 | pcg0007_39 | TRUE | 4 |
| ncgl2500 | pcg3381 | FALSE | 4 |
| ncgl2509 | pcg0007_39 | TRUE | 4 |
| ncgl2509 | pcg2613 | FALSE | 4 |
| ncgl2509 | pcg3121 | FALSE | 4 |
| ncgl2509 | pcg3381 | FALSE | 4 |
| ncgl2509 | wt | FALSE | 4 |
| ncgl2514 | pcg0007_39 | TRUE | 3 |
| ncgl2514 | pcg3121 | FALSE | 3 |
| ncgl2514 | pcg3381 | FALSE | 3 |
| ncgl2516 | pcg0007_39 | TRUE | 3 |
| ncgl2516 | pcg3381 | FALSE | 3 |
| ncgl2527 | pcg0007_39 | TRUE | 3 |
| ncgl2527 | pcg0007_39 | TRUE | 3 |
| ncgl2527 | pcg2613 | FALSE | 3 |
| ncgl2527 | pcg3381 | FALSE | 3 |
| ncgl2527 | pcg3381 | FALSE | 3 |
| ncgl2527 | wt | FALSE | 3 |
| ncgl2541 | pcg0007_119 | FALSE | 3 |
| ncgl2541 | pcg0007_39 | TRUE | 3 |
| ncgl2541 | pcg1860 | FALSE | 3 |
| ncgl2541 | pcg2613 | FALSE | 3 |
| ncgl2541 | pcg3121 | FALSE | 3 |
| ncgl2541 | pcg3381 | FALSE | 3 |
| ncgl2541 | wt | FALSE | 3 |
| ncgl2553 | pcg0007_39 | FALSE | 3 |
| ncgl2553 | pcg0007_39 | TRUE | 3 |
| ncgl2553 | pcg3121 | FALSE | 3 |
| ncgl2572 | pcg1860 | TRUE | 3 |
| ncgl2572 | pcg3121 | FALSE | 3 |
| ncgl2587 | pcg0007_119 | FALSE | 3 |
| ncgl2587 | pcg0007_39 | TRUE | 3 |
| ncgl2587 | pcg0755 | FALSE | 3 |
| ncgl2587 | pcg1860 | TRUE | 3 |
| ncgl2587 | pcg2613 | FALSE | 3 |
| ncgl2587 | pcg3121 | FALSE | 3 |
| ncgl2587 | pcg3381 | FALSE | 3 |
| ncgl2587 | wt | FALSE | 3 |
| ncgl2599 | pcg0007_39 | FALSE | 4 |
| ncgl2599 | pcg3381 | TRUE | 4 |
| ncgl2614 | pcg0007_39 | TRUE | 3 |
| ncgl2614 | pcg3121 | FALSE | 3 |
| ncgl2614 | pcg3381 | FALSE | 3 |
| ncgl2650 | pcg0007_39 | TRUE | 3 |
| ncgl2650 | pcg3121 | FALSE | 3 |
| ncgl2650 | pcg3381 | FALSE | 3 |
| ncgl2684 | pcg0007_39 | FALSE | 3 |
| ncgl2684 | pcg0007_39 | TRUE | 3 |
| ncgl2684 | pcg1860 | FALSE | 3 |
| ncgl2684 | pcg3121 | FALSE | 3 |
| ncgl2684 | pcg3381 | FALSE | 3 |
| ncgl2699 | pcg0007_39 | TRUE | 3 |
| ncgl2699 | pcg1860 | FALSE | 3 |
| ncgl2699 | pcg3121 | FALSE | 3 |
| ncgl2699 | pcg3381 | FALSE | 3 |
| ncgl2713 | pcg0007_39 | TRUE | 3 |
| ncgl2713 | pcg3381 | FALSE | 3 |
| ncgl2717 | pcg0007_39 | TRUE | 4 |
| ncgl2724 | pcg0007_39 | TRUE | 3 |
| ncgl2724 | pcg3121 | FALSE | 3 |
| ncgl2724 | pcg3381 | FALSE | 3 |
| ncgl2725 | pcg0007_39 | TRUE | 3 |
| ncgl2725 | pcg3381 | FALSE | 3 |
| ncgl2726 | pcg0007_39 | TRUE | 3 |
| ncgl2733 | pcg0007_39 | TRUE | 3 |
| ncgl2733 | pcg1860 | TRUE | 3 |
| ncgl2765 | pcg0007 | FALSE | 1 |
| ncgl2765 | pcg0007 | FALSE | 2 |
| ncgl2765 | pcg0007_119 | FALSE | 1 |
| ncgl2765 | pcg0007_119 | FALSE | 2 |
| ncgl2765 | pcg0007_39 | FALSE | 1 |
| ncgl2765 | pcg0007_39 | FALSE | 2 |
| ncgl2765 | pcg0755 | TRUE | 1 |
| ncgl2765 | pcg0755 | TRUE | 2 |
| ncgl2765 | pcg1860 | FALSE | 1 |
| ncgl2765 | pcg1860 | FALSE | 2 |
| ncgl2765 | pcg3121 | FALSE | 1 |
| ncgl2765 | pcg3121 | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2765 | wt | FALSE | 1 |
| ncgl2765 | wt | FALSE | 2 |
| ncgl2772 | pcg0007_119 | FALSE | 3 |
| ncgl2772 | pcg0007_39 | TRUE | 3 |
| ncgl2772 | pcg0755 | FALSE | 3 |
| ncgl2772 | pcg2613 | FALSE | 3 |
| ncgl2772 | pcg3121 | TRUE | 3 |
| ncgl2772 | pcg3381 | FALSE | 3 |
| ncgl2774 | pcg0007_39 | FALSE | 3 |
| ncgl2774 | pcg0007_39 | TRUE | 3 |
| ncgl2792 | pcg0007_39 | TRUE | 3 |
| ncgl2792 | pcg3121 | FALSE | 3 |
| ncgl2792 | pcg3381 | FALSE | 3 |
| ncgl2802 | pcg0007_39 | TRUE | 3 |
| ncgl2802 | pcg0007_39 | TRUE | 3 |
| ncgl2802 | pcg0755 | FALSE | 3 |
| ncgl2802 | pcg1860 | TRUE | 3 |
| ncgl2802 | pcg3381 | FALSE | 3 |
| ncgl2802 | pcg3381 | FALSE | 3 |
| ncgl2802 | wt | FALSE | 3 |
| ncgl2816 | pcg0007_39 | TRUE | 1 |
| ncgl2816 | pcg0007_39 | TRUE | 3 |
| ncgl2828 | pcg0007_39 | TRUE | 3 |
| ncgl2828 | pcg3121 | FALSE | 3 |
| ncgl2828 | pcg3381 | FALSE | 3 |
| ncgl2846 | pcg1860 | TRUE | 3 |
| ncgl2846 | pcg3121 | FALSE | 3 |
| ncgl2871 | pcg0007_119 | FALSE | 3 |
| ncgl2871 | pcg0007_39 | TRUE | 3 |
| ncgl2871 | pcg0755 | FALSE | 3 |
| ncgl2871 | pcg2613 | FALSE | 3 |
| ncgl2871 | pcg3121 | FALSE | 3 |
| ncgl2871 | pcg3381 | FALSE | 3 |
| ncgl2877 | pcg0007_39 | TRUE | 3 |
| ncgl2877 | pcg3121 | FALSE | 3 |
| ncgl2877 | pcg3381 | FALSE | 3 |
| ncgl2884 | pcg0007_39 | TRUE | 3 |
| ncgl2884 | pcg3121 | FALSE | 3 |
| ncgl2884 | pcg3381 | FALSE | 3 |
| ncgl2892 | pcg0007_39 | TRUE | 3 |
| ncgl2892 | pcg3381 | FALSE | 3 |
| ncgl2898 | pcg0007_39 | FALSE | 2 |
| ncgl2898 | pcg0007_39 | FALSE | 2 |
| ncgl2898 | pcg1860 | FALSE | 2 |
| ncgl2898 | pcg3121 | TRUE | 2 |
| ncgl2898 | pcg3381 | FALSE | 2 |
| ncgl2901 | pcg0007_119 | FALSE | 4 |
| ncgl2901 | pcg0007_39 | TRUE | 4 |
| ncgl2901 | pcg0755 | FALSE | 4 |
| ncgl2901 | pcg1860 | FALSE | 4 |
| ncgl2901 | pcg3121 | FALSE | 4 |
| ncgl2901 | pcg3381 | FALSE | 4 |
| ncgl2901 | wt | FALSE | 4 |
| ncgl2908 | pcg0007_39 | TRUE | 2 |
| ncgl2908 | pcg1860 | FALSE | 2 |
| ncgl2908 | pcg3121 | FALSE | 2 |
| ncgl2921 | pcg0007_119 | FALSE | 3 |
| ncgl2921 | pcg0007_39 | TRUE | 3 |
| ncgl2921 | pcg3121 | FALSE | 3 |
| ncgl2921 | pcg3381 | FALSE | 3 |
| ncgl2921 | wt | FALSE | 3 |
| ncgl2931 | pcg0007_119 | FALSE | 4 |
| ncgl2931 | pcg0007_39 | TRUE | 4 |
| ncgl2931 | pcg1860 | FALSE | 4 |
| ncgl2931 | pcg2613 | FALSE | 4 |
| ncgl2931 | pcg3121 | FALSE | 4 |
| ncgl2931 | pcg3381 | FALSE | 4 |
| ncgl2931 | wt | FALSE | 4 |
| ncgl2941 | pcg0007_39 | FALSE | 3 |
| ncgl2941 | pcg1860 | TRUE | 3 |
| ncgl2941 | pcg3121 | FALSE | 3 |
| ncgl2941 | pcg3381 | FALSE | 3 |
| ncgl2950 | pcg0007_39 | TRUE | 3 |
| ncgl2950 | pcg1860 | FALSE | 3 |
| ncgl2950 | pcg3121 | FALSE | 3 |
| ncgl2953 | pcg0007_39 | TRUE | 3 |
| ncgl2953 | pcg3381 | FALSE | 3 |
| ncgl2961 | pcg0007_39 | TRUE | 3 |
| ncgl2961 | pcg0755 | FALSE | 3 |
| ncgl2961 | pcg3121 | FALSE | 3 |
| ncgl2961 | pcg3121 | FALSE | 3 |
| ncgl2961 | pcg3381 | FALSE | 3 |
| ncgl2961 | pcg3381 | FALSE | 3 |
| ncgl2961 | pcg3381 | FALSE | 3 |
| ncgl2977 | pcg0007_39 | TRUE | 3 |
| ncgl2977 | pcg3121 | FALSE | 3 |
| ncgl2977 | pcg3381 | FALSE | 3 |
| ncgl2982 | pcg0007_39 | TRUE | 3 |
| ncgl2986 | pcg0007_39 | TRUE | 3 |
| ncgl2986 | pcg1860 | TRUE | 3 |
| ncgl2986 | pcg3381 | FALSE | 3 |
| ncgl2989 | pcg0007_39 | TRUE | 3 |
| ncgl2989 | pcg3121 | FALSE | 3 |
| ncgl2989 | pcg3381 | FALSE | 3 |

### Example 5: Allocation of Genes in the C. glutamicum Genome into Various Shells for Systematic Genome-Wide Perturbation

### Identification of Genes:

Identified genes were separated into four shells (1-4) based on the relevance of their impact to lysine production.

For shells 1 and 2, the genes in the genome of C. glutamicum were annotated by homology to the sequence of type strain ATCC 13032. The function of each gene in shells 1 and 2 was determined by using the KEGG pathway database. For shell 3, the genes in the genome of C. glutamicum were annotated using the RAST server. The function of each gene in shell 3 was determined using natural language search terms in the annotated description of each gene. These search terms were strings taken from the name of the metabolic area of interest.

Allocation into Each Shell:

The identified genes were allocated into shell 1 if they were involved in the conversion of direct metabolic intermediates between the substrate glucose and the product lysine. This included the transport of glucose into the cell, the transport of lysine out of the cell, and the enzymes involved in the conversion of carbon originally contained in glucose into each intermediate that ultimately was converted to lysine.

The identified genes were allocated into shell 2 if they were identified as being part of nitrogen metabolism, the TCA cycle, or the RNA degradasome KEGG pathway map. These areas of metabolism were chosen based on their relatedness to lysine production: Lysine contains significant nitrogen as compared to biomass, the TCA cycle generates energy for synthesis of lysine and biomass, and the RNA degradasome controls protein expression which is important to maximize for sufficient production during industrial fermentation.

The identified genes were allocated into shell 3 if they were identified as being part of cellular membrane transport, transcription, peptidoglycan biosynthesis, fatty acid biosynthesis, and biotin metabolism. These areas of metabolism are related to the production of lysine in industrial fermentation, but less so than the areas identified in shell 2. Transport is important to increase the productivity of each cell; altering genes related to transcription allows for the systematic modification of genes throughout the cell; peptidoglycan and fatty acid synthesis are involved in cell wall biosynthesis, which is the end point of one of the intermediates of lysine; biotin is an important cofactor for enzymes that are in the lysine metabolic pathway.

The identified genes were allocated into shell 4 if they did not fall into any of shells 1-3.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments described herein have been described herein for purposes of illustration, various modifications may be made without deviating from the spirit and scope described herein. Accordingly, the disclosure is not limited except as by the appended claims.

What is claimed is:

1. A host cell comprising a promoter polynucleotide sequence selected from the group consisting of SEQ ID NOs: 1 to 8 that is functionally linked to at least one heterologous ancillary target gene.

2. A host cell comprising:
a. a first promoter polynucleotide sequence that is functionally linked to at least one first heterologous target gene, wherein said at least one first heterologous target gene is a component of a biosynthetic pathway for producing a target biomolecule, wherein the target biomolecule is selected from the group consisting of amino acids, organic acids, proteins and polymers; and
b. a second promoter polynucleotide sequence selected from the group consisting of SEQ ID NOs: 1 to 8 that is functionally linked to at least one second heterologous target gene, wherein said at least one second heterologous target gene is an ancillary target gene.

3. The host cell according to claim 1, wherein said second promoter polynucleotide sequence is selected from the group consisting of SEQ ID NOs:1, 5 and 7.

4. The host cell according to any one of claims 1-3, wherein said ancillary target gene is a gene that is classified under GOslim term GO:0003674; GO:0003677; GO:0008150; GO:0034641; or GO:0009058.

5. The host cell according to claim 4, wherein said ancillary target gene is a gene that is classified under, or under at least, 2, 3, 4, or 5 of the following GOslim terms: GO:0003674; GO:0003677; GO:0008150; GO:0034641; and GO:0009058.

6. The host cell according to any one of claims 1-5, wherein said host cell is isolated.

7. The host cell according to any one of claims 1-6, wherein said ancillary target gene is not a component of a biosynthesis pathway comprising genes of one or more, or all, of the following KEGG entries: M00016; M00525; M00526; M00527; M00030; M00433 M00031; M00020; M00018; M00021; M00338; M00609; M00017; M00019; M00535; M00570; M00432; M00015; M00028; M00763; M00026; M00022; M00023; M00024; M00025; and M00040.

8. The host cell according to any one of claims 1-6, wherein said ancillary target gene is not asd, ask, aspB, cg0931, dapA, dapB, dapD, dapE, dapF, ddh, fbp, hom, icd, lysA, lysE, odx, pck, pgi, ppc, ptsG, pyc, tkt, or zwf, or an endogenous functional ortholog thereof in the host cell.

9. The host cell according to any one of claims 1-6, wherein said ancillary target gene is selected from the genes of one or more, or all, of the following KEGG entries: M00010, M00002, M00007, M00580, or M00005.

10. The host cell according to claim 2, wherein said at least one first heterologous target gene is a gene that is a component of an amino acid biosynthetic pathway.

11. The host cell according to claim 10, wherein said amino acid biosynthetic pathway is selected from the group consisting of:
the lysine biosynthesis pathway comprising genes of entry M00016 of the Kyoto Encyclopedia of Genes and Genomes (KEGG);
the lysine biosynthesis pathway comprising genes of KEGG entry M00525;
the lysine biosynthesis pathway comprising genes of KEGG entry M00526;
the lysine biosynthesis pathway comprising genes of KEGG entry M00527;
the lysine biosynthesis pathway comprising genes of KEGG entry M00030;
the lysine biosynthesis pathway comprising genes of KEGG entry M00433; and
the lysine biosynthesis pathway comprising genes of KEGG entry M00031.

12. The host cell according to claim 10, wherein said amino acid biosynthetic pathway is selected from the group consisting of the serine biosynthesis pathway comprising genes of KEGG entry M00020.

13. The host cell according to claim 10, wherein said amino acid biosynthetic pathway is selected from the group consisting of the threonine biosynthesis pathway comprising genes of KEGG entry M00018.

14. The host cell according to claim 10, wherein said amino acid biosynthetic pathway is selected from the group consisting of:
the cysteine biosynthesis pathway comprising genes of KEGG entry M00021;
the cysteine biosynthesis pathway comprising genes of KEGG entry M00338; and/or
the cysteine biosynthesis pathway comprising genes of KEGG entry M00609.

15. The host cell according to claim 10, wherein said amino acid biosynthetic pathway is selected from the group consisting of the methionine biosynthesis pathway comprising genes of KEGG entry M00017.

16. The host cell according to claim 10, wherein said amino acid biosynthetic pathway is selected from the group consisting of the valine/isoleucine biosynthesis pathway comprising genes of KEGG entry M00019.

17. The host cell according to claim 10, wherein said amino acid biosynthetic pathway is selected from the group consisting of:
the isoleucine biosynthesis pathway comprising genes of KEGG entry M00535; and/or
the isoleucine biosynthesis pathway comprising genes of KEGG entry M00570.

18. The host cell according to claim 10, wherein said amino acid biosynthetic pathway is selected from the group consisting of the leucine biosynthesis pathway comprising genes of KEGG entry M00432.

19. The host cell according to claim 10, wherein said amino acid biosynthetic pathway is selected from the group consisting of the proline biosynthesis pathway comprising genes of KEGG entry M00015.

20. The host cell according to claim 10, wherein said amino acid biosynthetic pathway is selected from the group consisting of:
the ornithine biosynthesis pathway comprising genes of KEGG entry M00028; and
the ornithine biosynthesis pathway comprising genes of KEGG entry M00763.

21. The host cell according to claim 10, wherein said amino acid biosynthetic pathway is selected from the group consisting of the histidine biosynthesis pathway comprising genes of KEGG entry M00026.

22. The host cell according to claim 10, wherein said amino acid biosynthetic pathway is selected from the group consisting of:
the shikimate biosynthesis pathway comprising genes of KEGG entry M00022;
the tryptophan biosynthesis pathway comprising genes of entry M00023;
the phenylalanine biosynthesis pathway comprising genes of KEGG entry M00024;
the tyrosine biosynthesis pathway comprising genes of KEGG entry M00025; and
the tyrosine biosynthesis pathway comprising genes of KEGG entry M00040.

23. The host cell of any one of claims 2-22, further comprising one or more additional second promoter polynucleotide sequences selected from the group consisting of SEQ ID NOs:1 to 8, each additional second promoter polynucleotide sequence functionally linked to at least one additional second heterologous target gene, wherein said at least one additional second heterologous target gene is an ancillary target gene.

24. The host cell according to claim 23, wherein said at least one second heterologous target gene and said at least one additional second heterologous target gene are part of the same metabolic pathway.

25. The host cell according to claim 24, wherein said at least one second heterologous target gene and said at least one additional second heterologous target gene are not part of the same metabolic pathway.

26. The host cell of any one of claims 2-22, further comprising one or more additional first promoter polynucleotide sequences selected from the group consisting of SEQ ID NOs:1 to 8, each additional first promoter polynucleotide sequence functionally linked to at least one additional first heterologous target gene, wherein the at least one first heterologous target gene and the at least one additional first heterologous target gene are in the same metabolic pathway.

27. The host cell according to any one of claims 1 to 26, which belongs to the genus Corynebacterium.

28. The host cell according to claim 27, which is Corynebacterium glutamicum.

29. The host cell according to any one of claims 1-28, wherein the ancillary target gene encodes an amino acid sequence selected from SEQ ID NOs:148-286.

30. The host cell according to any one of claims 1-29, wherein the ancillary target gene has a nucleotide sequence selected from SEQ ID NOs:9-147.

31. A method of producing a target biomolecule comprising culturing a host cell according to any one of claims 1 to 30 under conditions suitable for producing the biomolecule.

32. The method according to claim 31, wherein said biomolecule is an L-amino acid.

33. The method according to claim 32, wherein said L-amino acid is L-lysine.

34. A plurality of host cells comprising:
a. a first host cell comprising a first promoter polynucleotide sequence selected from a group of promoters comprising a plurality of promoters with incrementally increasing levels of promoter activity, wherein the first promoter polynucleotide is operably linked to a heterologous target gene, wherein the heterologous target gene is selected from genes within a pathway for production of a target biomolecule and heterologous ancillary target genes that are off the pathway for production of the target biomolecule;
b. a second host cell comprising a second promoter polynucleotide sequence selected from the group of promoters comprising the plurality of promoters with incrementally increasing levels of promoter activity, wherein the second promoter polynucleotide is functionally linked to a heterologous ancillary target gene, wherein the first and second promoter polynucleotide are different.

35. The plurality of host cells according to claim 34, wherein said plurality of host cells comprises at least 1×10⁶ cells.

36. The plurality of host cells according to claim 34 or 35, wherein said group of promoters comprising the plurality of promoters with incrementally increasing levels of promoter activity are constitutive promoters.

37. The plurality of host cells according to claim 34 or 35, wherein said group of promoters comprising the plurality of promoters with incrementally increasing levels of promoter activity are inducible promoters.

38. The plurality of host cells according to any one of claims 34-37, wherein said heterologous ancillary target gene operably linked to the second promoter polynucleotide is a shell 3 or shell 4 target gene; and/or wherein said first promoter polynucleotide is operably linked to a shell 3 or 4 heterolgous ancillary target gene.

39. The plurality of host cells according to any one of claims 34-38, wherein said heterologous ancillary target gene operably linked to said first and/or second promoter polynucleotide is not a component of a biosynthesis pathway comprising genes of one or more, or all, of the following KEGG entries: M00016; M00525; M00526; M00527; M00030; M00433 M00031; M00020; M00018; M00021; M00338; M00609; M00017; M00019; M00535; M00570; M00432; M00015; M00028; M00763; M00026; M00022; M00023; M00024; M00025; and M00040.

40. The plurality of host cells according to any one of claims 34-38, wherein said heterologous ancillary target gene operably linked to said first and/or second promoter polynucleotide is not asd, ask, aspB, cg0931, dapA, dapB, dapD, dapE, dapF, ddh, fbp, hom, icd, lysA, lysE, odx, pck, pgi, ppc, ptsG, pyc, tkt, or zwf, or an endogenous functional ortholog thereof in the host cell.

41. The plurality of host cells according to any one of claims 34-38, wherein said heterologous ancillary target gene operably linked to said first and/or second promoter polynucleotide is selected from the genes of one or more, or all, of the following KEGG entries: M00010, M00002, M00007, M00580, or M00005.

42. The plurality of host cells according to any one of claims 34-41, wherein said heterologous ancillary target gene operably linked to said first and/or second promoter polynucleotide is a gene classified under GOslim term GO:0003674; GO:003677; GO:0008150; GO:0034641; or GO:009058.

43. The plurality of host cells according to claim 42, wherein said heterologous ancillary target gene operably linked to said first and/or second promoter polynucleotide is a gene classified under, or under at least, 2, 3, 4, or 5 of the following GOslim terms GO:0003674; GO:003677; GO:0008150; GO:0034641; GO:009058.

44. The plurality of host cells according to claim 34-37, wherein said first promoter polynucleotide is operably linked to an on-pathway heterologous target gene for production of a target biomolecule, such as a heterologous target gene in shell 1 of a biosynthetic pathway for production of the target biomolecule.

45. The plurality of host cells according to claim 34-37, wherein said first promoter polynucleotide is operably linked to a heterologous shell 2 target gene.

46. The plurality of host cells according to any one of claims 34-45, wherein said pathway for production of target biomolecule is an amino acid biosynthetic pathway.

47. The plurality of host cells according to claim 46, wherein the amino acid biosynthetic pathway is selected from the group consisting of:
the lysine biosynthesis pathway comprising genes of entry M00016 of the Kyoto Encyclopedia of Genes and Genomes (KEGG);
the lysine biosynthesis pathway comprising genes of KEGG entry M00525;
the lysine biosynthesis pathway comprising genes of KEGG entry M00526;
the lysine biosynthesis pathway comprising genes of KEGG entry M00527;
the lysine biosynthesis pathway comprising genes of KEGG entry M00030;
the lysine biosynthesis pathway comprising genes of KEGG entry M00433; and
the lysine biosynthesis pathway comprising genes of KEGG entry M00031.

48. The plurality of host cells according to claim 46, wherein said amino acid biosynthetic pathway is selected from the group consisting of the serine biosynthesis pathway comprising genes of KEGG entry M00020.

49. The plurality of host cells according to claim 46, wherein said amino acid biosynthetic pathway is selected from the group consisting of the threonine biosynthesis pathway comprising genes of KEGG entry M00018.

50. The plurality of host cells according to claim 46, wherein said amino acid biosynthetic pathway is selected from the group consisting of:
the cysteine biosynthesis pathway comprising genes of KEGG entry M00021;
the cysteine biosynthesis pathway comprising genes of KEGG entry M00338; and/or the cysteine biosynthesis pathway comprising genes of KEGG entry M00609.

51. The plurality of host cells according to claim 46, wherein said amino acid biosynthetic pathway is selected from the group consisting of the methionine biosynthesis pathway comprising genes of KEGG entry M00017.

52. The plurality of host cells according to claim 46, wherein said amino acid biosynthetic pathway is selected from the group consisting of the valine/isoleucine biosynthesis pathway comprising genes of KEGG entry M00019.

53. The plurality of host cells according to claim 46, wherein said amino acid biosynthetic pathway is selected from the group consisting of:
a. the isoleucine biosynthesis pathway comprising genes of KEGG entry M00535; and/or
b. the isoleucine biosynthesis pathway comprising genes of KEGG entry M00570.

54. The plurality of host cells according to claim 46, wherein said amino acid biosynthetic pathway is selected from the group consisting of the leucine biosynthesis pathway comprising genes of KEGG entry M00432.

55. The plurality of host cells according to claim 46, wherein said amino acid biosynthetic pathway is selected from the group consisting of the proline biosynthesis pathway comprising genes of KEGG entry M00015.

56. The plurality of host cells according to claim 46, wherein said amino acid biosynthetic pathway is selected from the group consisting of:
a. the ornithine biosynthesis pathway comprising genes of KEGG entry M00028; and
b. the ornithine biosynthesis pathway comprising genes of KEGG entry M00763.

57. The plurality of host cells according to claim 46, wherein said amino acid biosynthetic pathway is selected from the group consisting of the histidine biosynthesis pathway comprising genes of KEGG entry M00026.

58. The plurality of host cells according to claim 46, wherein said amino acid biosynthetic pathway is selected from the group consisting of:
the shikimate biosynthesis pathway comprising genes of KEGG entry M00022;
the tryptophan biosynthesis pathway comprising genes of entry M00023;
the phenylalanine biosynthesis pathway comprising genes of KEGG entry M00024;
the tyrosine biosynthesis pathway comprising genes of KEGG entry M00025; and
the tyrosine biosynthesis pathway comprising genes of KEGG entry M00040.

59. The plurality of host cells according to any one of claims 34-58, wherein the first promoter polynucleotide and the second promoter polynucleotide are operably linked to the same heterologous ancillary target gene sequence.

60. The plurality of host cells according to any one of claims 34-59, wherein the plurality further comprises a third host cell comprising a third promoter polynucleotide sequence selected from the group of promoters comprising the plurality of promoters with incrementally increasing levels of promoter activity, wherein the third promoter is functionally linked to a heterologous target gene, and wherein the first, second, and third promoter are different.

61. The plurality of host cells according to claim 60, wherein the first, second, and third, promoter are operably linked to the same heterologous ancillary target gene.

62. The plurality of host cells according to claim 60 or 61, wherein the plurality further comprises a fourth host cell comprising a fourth promoter polynucleotide sequence selected from the group of promoters comprising the plurality of promoters with incrementally increasing levels of promoter activity, wherein the fourth promoter is functionally linked to a heterologous target gene, and wherein the first, second, third, and fourth promoter are different.

63. The plurality of host cells according to claim 62, wherein the first, second, third, and fourth promoter are operably linked to the same heterologous ancillary target gene.

64. The plurality of host cells according to claim 62 or 63, wherein the plurality further comprises a fifth host cell comprising a fifth promoter polynucleotide sequence selected from the group of promoters comprising the plurality of promoters with incrementally increasing levels of promoter activity, wherein the fifth promoter is functionally linked to a heterologous target gene, and wherein the first, second, third, fourth, and fifth promoter are different.

65. The plurality of host cells according to claim 64, wherein the first, second, third, fourth, and fifth promoter are operably linked to the same heterologous ancillary target gene.

66. The plurality of host cells according to claim 64 or 65, wherein the plurality further comprises a sixth host cell comprising a sixth promoter polynucleotide sequence selected from the group of promoters comprising the plurality of promoters with incrementally increasing levels of promoter activity, wherein the sixth promoter is functionally linked to a heterologous target gene, and wherein the first, second, third, fourth, fifth, and sixth promoter are different.

67. The plurality of host cells according to claim 66, wherein the first, second, third, fourth, fifth, sixth, and seventh promoter are operably linked to the same heterologous ancillary target gene.

68. The plurality of host cells according to claim 66 or 67, wherein the plurality further comprises an eighth host cell comprising an eighth promoter polynucleotide sequence selected from the group of promoters comprising the plurality of promoters with incrementally increasing levels of promoter activity, wherein the eighth promoter is functionally linked to a heterologous target gene, and wherein the first, second, third, fourth, fifth, sixth, seventh, and eighth promoter are different.

69. The plurality of host cells according to claim 68, wherein the first, second, third, fourth, fifth, sixth, seventh, and eighth promoter are operably linked to the same heterologous ancillary target gene.

70. The plurality of host cells according to any one of claims 34-70, wherein said host cells are Corynebacterium host cells.

71. The plurality of host cells according to claim 71, wherein said Corynebacterium host cells are Corynebacterium glutamicum host cells.

72. The plurality of host cells according to any one of claims 34-71, wherein said host cells further comprise a promoter polynucleotide sequence operably linked to a heterologous target gene directly involved in a selected metabolic pathway for production of the target molecule.

73. A method comprising culturing a plurality of host cells according to any one of claims 34-72.

74. A plurality of transformed host cells comprising a combination of promoter polynucleotides functionally linked to at least one heterologous ancillary target gene, wherein said combination of promoter polynucleotides comprises a plurality of promoters with incrementally increasing levels of promoter activity.

75. The transformed host cells according to claim 74, wherein said combination of promoter polynucleotides comprises at least one first promoter polynucleotide comprising a sequence selected from the group consisting of: SEQ ID NO:1, SEQ ID NO:5, and SEQ ID NO:7, and at least one second promoter polynucleotide comprising a sequence selected from the group consisting of: SEQ ID NO:2, SEQ ID NO:3, SEQ ID NO:4, SEQ ID NO:6, and SEQ ID NO:8.

76. The transformed host cells according to claim 74 or 75, wherein each promoter polynucleotide is functionally linked to a different heterologous target gene.

77. The transformed host cells according to claim 74 or 75, wherein each promoter polynucleotide is functionally linked to the same heterologous ancillary target gene.

78. A method comprising culturing a plurality of host cells according to any one of claims 74-77.

79. A method for increasing production of a target biomolecule, the method comprising:
a. providing a plurality of host cells, wherein the plurality of host cells comprises plurality of heterologous promoters with incrementally increasing levels of promoter activity, wherein the promoters of the plurality are each operably linked to a heterologous target gene and at least one promoter of the plurality of promoters is operably linked to a heterologous ancillary target gene;
b. culturing the plurality of host cells under conditions suitable to produce the target biomolecule; and
c. identifying a host cell from the plurality of host cells that exhibits increased production of target biomolecule as compared to a control cell.

80. The method of claim 79, wherein the method further comprises isolating the identified host cell from other host cells of the plurality.

81. The method of claim 80, wherein the method comprises storing the isolated host cell.

82. The method of claim 80, wherein the method comprises expanding the isolated host cell.

83. The method of any one of claims 79-82, wherein the plurality of host cells comprises at least a first and a second host cell, wherein the first and second host cell are transformed with a different promoter selected from the plurality of heterologous promoters with incrementally increasing levels of promoter activity and wherein the different promoters are operably linked to the same heterologous ancillary target gene.

84. The method of claim 83, wherein the plurality of host cells further comprises a third host cell, wherein the first, second, and third host cell are each transformed with a different promoter selected from the plurality of heterologous promoters with incrementally increasing levels of promoter activity and wherein the different promoters are operably linked to the same heterologous ancillary target gene.

85. The method of claim 84, wherein the plurality of host cells further comprises a fourth host cell, wherein the first, second, third, and fourth host cell are each transformed with a different promoter selected from the plurality of heterologous promoters with incrementally increasing levels of promoter activity and wherein the different promoters are operably linked to the same heterologous ancillary target gene.

86. The method of claim 85, wherein the plurality of host cells further comprises a fifth host cell, wherein the first, second, third, fourth, and fifth host cell are each transformed with a different promoter selected from the plurality of heterologous promoters with incrementally increasing levels of promoter activity and wherein the different promoters are operably linked to the same heterologous ancillary target gene.

87. The method of claim 86, wherein the plurality of host cells further comprises a sixth host cell, wherein the first, second, third, fourth, fifth, and sixth host cell are each transformed with a different promoter selected from the plurality of heterologous promoters with incrementally increasing levels of promoter activity and wherein the different promoters are operably linked to the same heterologous ancillary target gene.

88. The method of claim 87, wherein the plurality of host cells further comprises a seventh host cell, wherein the first, second, third, fourth, fifth, sixth, and seventh host cell are each transformed with a different promoter selected from the plurality of heterologous promoters with incrementally increasing levels of promoter activity and wherein the different promoters are operably linked to the same heterologous ancillary target gene.

89. The method of claim 88, wherein the plurality of host cells further comprises an eighth host cell, wherein the first, second, third, fourth, fifth, sixth, seventh, and eighth host cell are each transformed with a different promoter selected from the plurality of heterologous promoters with incrementally increasing levels of promoter activity and wherein the different promoters are operably linked to the same heterologous ancillary target gene.

90. The method of any one of claims 79-89, wherein the heterologous ancillary target gene is a shell 3 and/or shell 4 target gene.

91. The method of any one of claims 79-90, wherein the providing comprises transforming a plurality of host cells with a recombinant vector library comprising the plurality of promoters with incrementally increasing levels of promoter activity operably linked to the heterologous target genes.
